(19) United States
(12) Patent Application Publication
(10) Pub. No.: US 2023/0340488 A1
(43) Pub. Date: Oct. 26, 2023

(54) TREATMENT OF SKIN DISEASES AND DISORDERS USING INHIBITORS OF KALLIKREIN-RELATED PEPTIDASES (KLK)

(71) Applicants: Exicure Operating Company, Chicago, IL (US); SevenScore Pharmaceuticals, LLC, New York, NY (US)

(72) Inventors: Bart Anderson, Chicago, IL (US); Michael Mutolo, Chicago, IL (US)

(73) Assignees: Exicure Operating Company, Chicago, IL (US); SevenScore Pharmaceuticals, LLC, New York, NY (US)

(21) Appl. No.: 18/019,857

(22) PCT Filed: Aug. 6, 2021
(86) PCT No.: PCT/US2021/045084

§ 371 Date: Feb. 6, 2023

Related U.S. Application Data
(60) Provisional application No. 63/114,409, filed on Nov. 16, 2020.
(60) Provisional application No. 63/062,951, filed on Aug. 7, 2020.

(51) Int. Cl. C12N 15/113; A61K 45/06

(52) U.S. Cl. CPC C12N 15/1137; A61K 45/06; C12N 2310/3515; C12N 2310/3231; C12N 2310/11; C12N 2310/344; C12N 2320/31; C12Y 304/21008; C12N 2310/341

(57) ABSTRACT

Provided herein are compositions and methods for inhibiting the expression and/or activity of kallikrein-related peptidase (KLK) (e.g., KLK5 and KLK7) genes and/or KLK (e.g., KLK5 and KLK7) gene products using oligonucleotide-based therapy. In some embodiments, the compositions are spherical nucleic acids (SNAs) targeting a region of a KLK5 gene and/or gene product and a second synthetic oligonucleotide targeting a region of a KLK7 gene and/or a KLK7 gene product. Such compositions and methods are useful, for instance, in the treatment, prevention, and/or amelioration of diseases or disorders associated with the dysregulation of KLK function.

### RELATED APPLICATIONS

This application claims the benefit of the filing date under 35 U.S.C. § 119(e) of U.S. Provisional Application Ser. No. 63/062,951, entitled “TREATMENT OF SKIN DISEASES AND DISORDERS USING INHIBITORS OF KALLIKREIN (KLK)-RELATED PEPTIDASES”, filed on Aug. 7, 2020, and of U.S. Provisional Application Ser. No. 63/114,409, entitled “TREATMENT OF SKIN DISEASES AND DISORDERS USING INHIBITORS OF KALLIKREIN-RELATED PEPTIDASES (KLK)”, filed on Nov. 16, 2020, the contents of each of which are incorporated herein by reference in their entireties.

### BACKGROUND

Netherton syndrome (NS) is a devastating genetic disease caused by mutations in serine protease inhibitor of Kazal type (STINK) 5. SPIN K5 encodes the serine protease inhibitor lympho-epithelial Kazal-type-related inhibitor (LEKTI), which inhibits the activity of kallikrein-related peptidase (KLK) 5 and KLK7. NS has an estimated prevalence of one in 50,000-200,000, from which 1,600-6,500 patients and another 3,700-15,000 patients are estimated to suffer from NS in the United States and in Europe, respectively.

### SUMMARY

The present disclosure relates, according to some aspects, to compositions and uses thereof that decrease the expression of certain kallikrein-related peptidase (KLK) genes for the treatment of certain diseases and disorders, such as chronic skin diseases and disorders, including, but not limited to, Netherton syndrome (NS) and atopic dermatitis (AD). As disclosed herein, compositions, such as oligonucleotides (e.g., synthetic oligonucleotides) and spherical nucleic acids (SNAs) targeting KLK genes (e.g., a KLK5 gene, a KLK7 gene, and/or related KLK5 and KLK7 gene products) decrease the expression of KLK genes (e.g., mRNA levels, protein levels, etc.) by decreasing KLK gene expression and/or KLK protein to levels that promote either partially or completely one or more beneficial phenotype(s). The compositions disclosed herein can be used for the treatment, amelioration and/or elimination of one or more, or all, characteristics, conditions, and/or symptoms associated with, for instance, chronic skin diseases and disorders, including, but not limited to NS and AD.

In some embodiments, an SNA disclosed herein includes a first synthetic oligonucleotide which targets a KLK5 gene and/or a KLK5 gene product and a second synthetic oligonucleotide which targets a KLK7 gene and/or a KLK7 gene product, thereby forming a bispecific SNA which decreases KLK5 mRNA and/or KLK5 protein, as well as KLK7 mRNA and/or KLK7 protein to levels that promote either partially or completely one or more beneficial phenotype(s) for the treatment, amelioration and/or elimination of chronic skin diseases and disorders.

According to some aspects, synthetic oligonucleotides, or pharmaceutically acceptable salts thereof, are contemplated herein. In some embodiments, the synthetic oligonucleotide comprises a nucleic acid sequence complementary to or sufficiently complementary to a region of a KLK gene and/or to a region of a KLK gene product, and at least one locked nucleic acid (LNA) modification or LNA modified nucleoside, or a pharmaceutically acceptable salt thereof.

In some embodiments, the KLK gene is a KLK5 gene. In some embodiments, the KLK gene product is a KLK5 gene product. In some embodiments, the KLK gene is a KLK7 gene. In some embodiments, the KLK gene product is a KLK7 gene product.

In some embodiments, the synthetic oligonucleotide comprises a nucleic acid sequence complementary to or sufficiently complementary to a region of a kallikrein-related peptidase (KLK) 7 gene and/or to a region of a KLK7 gene product, and at least one modification chosen from a base modification, a sugar modification or an internucleoside linkage modification, or a pharmaceutically acceptable salt thereof.

In some embodiments, the synthetic oligonucleotide or the nucleic acid sequence is indirectly attached to a molecular species, in some embodiments, the molecular species is indirectly attached to the 3′-end of the synthetic oligonucleotide or the nucleic acid sequence. In some embodiments, the molecular species is indirectly attached to the 5′-end of the synthetic oligonucleotide or the nucleic acid sequence, in some embodiments, the molecular species comprises, consists essentially of, or consists of a cholesterol or a tocopherol. In some embodiments, the molecular species comprises, consists essentially of, or consists of a cholesterol. In some embodiments, the molecular species comprises, consists essentially of, or consists of (N-cholesteryl-3-aminopropyl)-triethyleneglycol-glyceryl-1-O-phosphodiester (CholTEG).

In some embodiments, the synthetic oligonucleotide further comprises a spacer. In some embodiments, the spacer comprises, consists essentially of, or consists of an oligoethylene. In some embodiments, the oligoethylene is hexaethyleneglycol (HEG). In some embodiments, the spacer comprises, consists essentially of, or consists of HEG and triethyleneglycol (TEG). In some embodiments, the spacer comprises, consists essentially of, or consists of hexa(ethyleneglycopphosphodiester-hexa(ethyleneglycoOphosphodiester (HEG-HEG). In some embodiments, the molecular species is indirectly attached to the synthetic oligonucleotide or the nucleic acid sequence through the spacer.

In some embodiments, the synthetic oligonucleotide comprises at least one phosphorothioate internucleoside linkage. In some embodiments, the synthetic oligonucleotide comprises two or more phosphorothioate internucleoside linkages, or each internucleoside linkage of the synthetic oligonucleotide is a phosphorothioate internucleoside linkage.

In some embodiments, the synthetic oligonucleotide comprises at least two modifications or modified nucleoside(s). In some embodiments, the at least two modifications or modified nucleosides are at least one of a modification or modified nucleoside chosen from a 2′-O-methyl modification (2′-O-Me) or 2′-O-Me modified nucleoside, a 2′-O-methoxyethyl (2′-MOE) modification or 2′-MOE modified nucleoside, a 2′-O-methoxyethoxy-5-methyl (5-Me-MOE) modification or 5-Me-MOE modified nucleoside, an LNA modification or LNA modified nucleoside, a 5-methyl (5-Me) modification or 5-Me modified nucleoside, a 5-methyl LNA modified nucleoside, a 7-deaza modification or 7-deaza modified nucleoside, and a 7-deaza-2′-O-methyl (7deazaOM) modification or 7deazaOM modified nucleoside.

In some embodiments, the synthetic oligonucleotide further comprises at least one modification or modified nucleoside chosen from a 5-Me modification or a 5-Me modified nucleoside, a. 7-deaza modification or 7-deaza modified nucleoside and a phosphorothioate internucleoside linkage. In some embodiments, the synthetic oligonucleotide comprises an LNA modification or an LNA modified nucleoside, a 5-Me modification or 5-Me modified nucleoside, and a phosphorothioate internucleoside linkage. In some embodiments, the synthetic oligonucleotide comprises five 5-Me modified nucleosides and six LNA modified nucleosides. In some embodiments, the synthetic oligonucleotide comprises an LNA modification or an LNA modified nucleoside, a 5-Me modification or 5-Me modified nucleoside, a 7-deaza modification or 7-deaza modified nucleoside, and a phosphorothioate internucleoside linkage. In some embodiments, the synthetic oligonucleotide comprises one 5-Me modification or 5-Me modified nucleoside, one 7-deaza modification or 7-deaza modified nucleoside and six LNA modifications or LNA modified nucleosides.

In some embodiments, the LNA modified nucleoside comprises, consists essentially of, or consists of (2′-O, 4′-C methylene)-Adenosine, 5-methyl-(2′-O, 4′-C methylene)-Cytidine, (2′-O, 4′-C methylene)-Guanosine, or 5-methyl-(2′-O, 4′-C methylene)-Uridine.

In some embodiments, the region is within an exon. In some embodiments, the region is within exon 6 of a KLK5 gene or a KLK5 gene product. In some embodiments, the region is within exon 5 of a KLK7 gene or a KLK7 gene product. In some embodiments, the region is within exon 1, exon 3, exon 4 or exon 6 of a KLK7 gene or a KLK7 gene product. In some embodiments, the region is within both exons 5 and 6 of a KLK7 gene or a KLK7 gene product.

In some embodiments, the region is within a 5′-untranslated region (UTR) or a 3′-UTR.

In some embodiments, the region is within a coding sequence.

In some embodiments, the nucleic acid sequence is 10 to 30 nucleobases or nucleosides in length. In some embodiments, the nucleic acid sequence is 15 to 22 nucleobases or nucleosides in length. In some embodiments, the nucleic acid sequence is 17 nucleobases or nucleosides in length. In some embodiments, the nucleic acid sequence is 20 nucleobases or nucleosides in length.

In some embodiments, the nucleic acid sequence comprises, consists essentially of, or consists of GAGGTCAGAGGGAAAGG (SEQ ID NO: 3998).

In some embodiments, the synthetic oligonucleotide comprises, consists essentially of, or consists of /CholTEG//iSp18//iSp18/lG*lA*lG*G*T*/iMe-dC/*A*G*A*G*/7deazaG/*G*A*AlA*lG*lG (SEQ ID NO: 7616), wherein lA, lG, A, T, G, iMe-dC, 7deazaG, *, iSp18 and CholTEG are defined as in Table 24.

In some embodiments, the nucleic acid sequence comprises, consists essentially of, or consists of CAGACCCTGAGTCCAGGAGA (SEQ ID NO: 1312).

In some embodiments, the synthetic oligonucleotide comprises, consists essentially of, or consists of /5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), wherein 5-Me-lC, lA, lC, A, T, G, iMe-dC, *, iSp18 and 3CholTEG are defined as in Table 24.

In some embodiments, the nucleic acid sequence comprises, consists essentially of, or consists of GAGACGCAACCCCCGCCCTG (SEQ ID NO: 3118).

In some embodiments, the synthetic oligonucleotide comprises, consists essentially of, or consists of lG*lA*lG*A*/iMe-dC/*/iMe-dCl*A*A*/iMe-dC/*/iMe-dC/*/iMe-dC/*/iMe-dC/*/iMe-dCl*G*/iMe-dC/G*/iMe-dC/*/5-Me-lC/*lT*lG/iSp18//iSp18//3CholTEG/ (SEQ ID NO: 7836), wherein 5-Me-lC lA, lG, lT, A, G, iMe-dC, *, iSp18, and 3CholTEG are defined as in Table 24.

According to some aspects, a pharmaceutical composition comprising a synthetic oligonucleotide disclosed herein, or a pharmaceutically acceptable salt thereof, is contemplated herein.

According to some aspects, a spherical nucleic acid (SNA) is contemplated herein. In some embodiments, the SNA comprises a core and an oligonucleotide shell, wherein the oligonucleotide shell comprises a first synthetic oligonucleotide comprising a first nucleic acid sequence complementary to or sufficiently complementary to a region of a first kallikrein-related peptidase (KLK) gene that is a KLK5 gene and/or to a region of a first KLK gene product that is a KLK5 gene product, and a second synthetic oligonucleotide comprising a second nucleic acid sequence complementary to or sufficiently complementary to a region of a second KLK gene that is a KLK7 gene and/or to a region of a second KLK gene product that is a KLK gene product.

In some embodiments, the region of the first KLK gene and/or the region of the first KLK gene product is within exon 6 of a KLK5 gene or a KLK5 gene product.

In some embodiments, the region of the second. KLK gene and/or the region of the second KLK gene product is within exon 5 of a KLK7 gene or a KLK7 gene product.

In some embodiments, the region of the first KLK gene and/or the region of the first KLK gene product is within exon 6 of a KLK5 gene or a KLK5 gene product and the region of the second KLK gene and/or the region of the second KLK gene product is within exon 5 of a KLK7 gene or a KLK7 gene product.

In some embodiments, the first synthetic oligonucleotide is a synthetic oligonucleotide disclosed herein and the second synthetic oligonucleotide is a synthetic oligonucleotide disclosed herein.

In some embodiments, the first synthetic oligonucleotide or nucleic acid sequence is indirectly attached to a first molecular species and the second synthetic oligonucleotide or nucleic acid sequence is indirectly attached to a second molecular species.

In some embodiments, the first molecular species is indirectly attached to one end of the first synthetic oligonucleotide or nucleic acid sequence and the second molecular species is indirectly attached to one end of the second synthetic oligonucleotide or nucleic acid sequence. In some embodiments, the first molecular species is indirectly attached to the 3′-end of the first synthetic oligonucleotide or nucleic acid sequence and the second molecular species is indirectly attached to the 5′-end of the second synthetic oligonucleotide or nucleic acid sequence.

In some embodiments, the first synthetic oligonucleotide is anchored to the surface of the core through the first molecular species and the second synthetic oligonucleotide is anchored to the surface of the core through the second molecular species.

In some embodiments, the oligonucleotide shell comprises a plurality of the first synthetic oligonucleotides to form a first population of synthetic oligonucleotides and a plurality of the second synthetic oligonucleotides to form a second population of synthetic oligonucleotides, wherein each of the first synthetic oligonucleotides is anchored to the surface of the core through the first molecular species and each of the second synthetic oligonucleotides is anchored to the surface of the core through the second molecular species.

In some embodiments, the first nucleic acid sequence comprises, consists essentially of, or consists of CAGACCCTGAGTCCAGGAGA (SEQ ID NO: 1312), and the second nucleic acid sequence comprises, consists essentially of, or consists of GAGGTCAGAGGGANAGG (SEQ ID NO: 3998).

In some embodiments, the first synthetic oligonucleotide comprises, consists essentially of, or consists of /5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3Chol (SEQ ID NO: 7834), and the second synthetic oligonucleotide comprises, consists essentially of, or consists of /CholTEG//iSp18//iSp18/lG*lA*lG*G*T*/iMe-dC/*A*G*A*G*/7deazaG/*G*A*A*lA*lG*lG (SEQ ID NO: 7616), wherein 5-Me-lC, lA, lG, A, T, G, iMe-dC, 7deazaG, *, iSp18, CholTEG and 3CholTEG are defined as in Table 24.

In some embodiments, the first synthetic oligonucleotide comprises, consists essentially of, or consists of a synthetic oligonucleotide disclosed herein and the second synthetic oligonucleotide comprises, consists essentially of, or consists of a synthetic oligonucleotide disclosed herein.

In some embodiments, the first nucleic acid sequence comprises, consists essentially of, or consists of GAGACGCAACCCCCGCCCTG (SEQ ID NO: 3118), and the second nucleic acid sequence comprises, consists essentially of, or consists of GAGGTCAGAGGGAAAGG (SEQ ID NO: 3998).

In some embodiments, the first synthetic oligonucleotide comprises, consists essentially of, or consists of lG*lA*lG*A*/iMe-dC/*G*/iMe-dC/*A*A*/iMe-dC/*/iMe-dC/*/iMe-dC/*/iMe-dC/*/iMe-dC/*G*/iMe-dC/*/iMe-dC/*/5-Me-lC/*lT*lG/iSp18//iSp18//3CholTEG/ (SEQ ID NO: 7836), and the second synthetic oligonucleotide comprises, consists essentially of, or consists of /CholTEG//iSp18//iSp18/lG*lA*lG*G*T*/iMe-dC/*A*G*A*G*/7deazaG/*G*A*A*lA*lG*lG (SEQ ID NO: 7616) wherein lG, lA, lT, 5-Me-lC, A, G, T, 7deazaG, *, iSp18, CholTEG and 3CholTEG are defined as in Table 24.

In some embodiments, the first synthetic oligonucleotide comprises, consists essentially of, or consists of the synthetic oligonucleotide disclosed herein and the second synthetic oligonucleotide comprises, consists essentially of, or consists of the synthetic oligonucleotide disclosed herein.

In some embodiments, the core is a hollow core or solid core. In some embodiments, the hollow core is a liposome core.

In some embodiments, the oligonucleotide shell comprises, consists essentially of, or consists of 20 to 50 total first synthetic oligonucleotides and second synthetic oligonucleotides. In some embodiments, the oligonucleotide shell comprises, consists essentially of, or consists of 25 to 35 total first synthetic oligonucleotides and synthetic oligonucleotides. In some embodiments, the oligonucleotide shell comprises, consists essentially of, or consists of or about 30 total first synthetic oligonucleotides and second synthetic oligonucleotides. In some embodiments, the oligonucleotide shell comprises, consists essentially of, or consists of or about 15 first synthetic oligonucleotides and of or about 15 second synthetic oligonucleotides.

In some embodiments, the core is a liposome core comprising or consisting of lipid molecules, and wherein the first population of synthetic oligonucleotides and the second population of synthetic oligonucleotides are at a molar ratio of or about 50 to 1 of lipid molecules to the first population of synthetic oligonucleotides and the second population of synthetic oligonucleotides. In some embodiments, the core is a liposome core comprising lipid molecules, and wherein the first population of synthetic oligonucleotides and the second population of synthetic oligonucleotides are at a molar ratio of or about 50 to 0.5 to 0.5 of lipid molecules to the first population of synthetic oligonucleotides to the second population of synthetic oligonucleotides.

According to some aspects a pharmaceutical composition comprising an SNA disclosed herein, or a pharmaceutically acceptable salt thereof, is contemplated herein.

According to some aspects, methods of decreasing KLK mRNA levels in a cell are contemplated herein. In some embodiments, the method comprises contacting a cell comprising a KLK gene and/or a KLK gene product with a synthetic oligonucleotide disclosed herein, an SNA disclosed herein, or a pharmaceutical composition disclosed herein, to decrease KLK mRNA levels in the cell relative to a reference level.

In some embodiments, the KLK mRNA levels are KLK5 mRNA levels, KLK7 mRNA levels, or both KLK5 mRNA levels and KLK7 mRNA levels.

In some embodiments, the cell comprises a KLK5 gene, a KLK7 gene, or a KLK5 gene and a KLK7 gene.

In some embodiments, the decreased KLK5 mRNA levels, KLK7 mRNA levels, or KLK5 mRNA levels and KLK7 mRNA levels result in decreased KLK5 protein levels, KLK7 protein levels, or both KLK5 protein levels and KLK7 protein levels in the cell relative to a reference level.

In some embodiments, the cell is a skin cell. In some embodiments, the cell is at least one of an epithelial cell, a dermal cell, a keratinocyte, an immune cell, a basal cell, an inner root sheath cell, an external root sheath cell, a sebaceous gland cell, and a sweat gland cell. In some embodiments, the cell is a keratinocyte.

According to some aspects, methods of treating a disease or disorder in a subject are contemplated herein. In some embodiments, the method of treating a disease or disorder in a subject comprises administering to a subject in order to treat the disease or disorder in the subject an effective amount of a synthetic oligonucleotide disclosed herein, an SNA disclosed herein, or a pharmaceutical composition disclosed herein.

In some embodiments, the disease or disorder is an ichthyosis disease or disorder.

In some embodiments, the subject has mutations in two serine peptidase inhibitor, Kazal type 5 (SPINK5) alleles.

In some embodiments, the disease or disorder is a chronic skin disease or disorder.

In some embodiments, the disease or disorder is Netherton syndrome (NS) or atopic dermatitis. In some embodiments, the disease or disorder is NS.

In some embodiments, the decrease in KLK5 mRNA levels, KLK7 mRNA levels, or in both KLK5 mRNA levels and KLK7 mRNA levels and/or the decrease in KLK5 protein levels, KLK7 protein levels, or both KLK5 protein levels and KLK7 protein levels in the subject is relative to a reference level.

In some embodiments, the KLK5 and/or KLK7 mRNA and/or protein levels are decreased in one or more cells in the subject, wherein the one or more cells in the subject are at least one of a skin cell, an epithelial cell, a dermal cell, a keratinocyte, an immune cell, a basal cell, an inner root sheath cell, an external root sheath cell, a sebaceous gland cell, and a sweat gland cell. In some embodiments, the KLK5 and/or KLK7 mRNA and/or protein levels are decreased in a keratinocyte.

In some embodiments, the synthetic oligonucleotide disclosed herein, the SNA disclosed herein, or the pharmaceutical composition disclosed herein ameliorate or eliminate one or more symptoms or conditions associated with the disease or disorder in the subject, and wherein the symptom or condition is at least one of erythroderma, xeroderma, desquamation, pruritus, skin infections, septicemia, inflammation, ichthyosis linearis circumflexa, bamboo hair, eczema, asthma, allergies, hypernatremia, and dehydration.

In some embodiments, the method further comprises the administration of a second therapeutic agent.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### DETAILED DESCRIPTION

Chronic skin diseases affect large portion of the world population. It is estimated that approximately 60% of the worldwide population has some sort of a dermatological disorder, with 25% of this number requiring medical attention (Eissa, et al. Biol Chem (2008) 389:669-80). Many research efforts have been made to better understand the complexity in different signaling pathways that manifest into different chronic skin diseases, as well as to develop innovative drug delivery systems that can allow the drug to penetrate through the protective outer skin to be localized in the epidermis layer.

Human tissue kallikreins (KLKs) are secreted serine protease endopeptidases, and the KLK enzymes are co-localized in the upper stratum granulosum and stratum corneum of human epidermis and in associated appendages such as hair follicle epithelia and sweat glands (Eissa, et al. Biol Chem (2008) 389:669-80). Several KLKs are crucially involved in the regulation of skin desquamation and inflammation. Abnormal activation of these KLKs occurs in several skin diseases such as Netherton Syndrome (NS), atopic dermatitis (AD), rosacea and psoriasis (Eissa, et al. Biol Chem (2008) 389:669-80; Nauroy, et al. Matrix Biology Plus (2020) 6-7:100019; Chen, et al. International Journal of Dermatology and Venereology (2019) 2:3).

More specifically, KLK5 and KLK7 play a key role in degrading desmosomal proteins that are responsible for the structural integrity of the epidermis, of which such degradation of desmosomes is necessary for the shedding of old cells from the skin surface and maintenance of a healthy epidermis, a process known as desquamation (Chen, et al. International Journal of Dermatology and Venereology (2019) 2:3; Simon, et al. J Biol Chem (2001) 276:20292-9; Caubet, et al. J Invest Dermatol (2004) 122:1235-44). For example, the regulation of KLK5 and KLK7 completely rescues the epidermal barrier and the postnatal lethality in NS mice allowing them to reach adulthood with fully functional skin and normal hair growth (Kasparek et al., PLOS Genetics (2017) 13(1l):e1006566). The overactivity of KLK5 was previously found to be responsible for the exacerbation of rosacea and dermatoses such as pruritus and atopic dermatitis (Matsubara, et al. Molecules (2017) 22:1829).

NS is a rare genetic disorder, inherited in an autosomal recessive pattern, which affects the skin, hair and immune system. NS causes a variety of symptoms, including red scaly skin, outbreaks of circular scaly rashes, bamboo hair (thin, fragile hair), and excessive immune reactions such as hay fever, asthma, itchy skin, and eczema. Dehydration and frequent infections resulting from insufficiency of the skin barrier are common and can be serious or life-threatening. Patients with NS are predisposed to a variety of infections, including viral (e.g., herpes simplex virus, human papilloma virus, etc.) and bacterial infections. Infections associated with NS often lead to septicemia, which can be life-threatening.

Diagnosis of NS is based on clinical examination, symptoms, and genetic testing.

Treatment options, among others, include symptom management, such as topical treatment with mild moisturizers, skin barrier repair formulas including ceramides or cholesterol, and low potency forms of topical steroids. While systemic immunomodulatory medications have been tested, their use is typically avoided as these medications often have limited benefits and are associated with severe side-effects.

NS is a rare hereditary disorder caused by autosomal recessive mutations in serine protease inhibitor of Kazal type 5 (SPINK5). It occurs when two mutated alleles of SPINK5 are inherited. SPINK5 encodes lympho-epithelial Kazal-type-related inhibitor (LEKTI), an inhibitor of protease activity. LEKTI inhibits kallikrein-related peptidases (KLKs), including KLK5, KLK7 and KLK14, particularly within the skin. These three proteases possibly regulate cell shedding (desquamation), given their ability to degrade proteins which form the extracellular component of cell junctions in the stratum corneum. There are approximately 150 cases reported in the medical literature, but the true number of affected individuals may be much higher due to diagnostic difficulties and overlapping symptoms with common AD and other congenital ichthyoses.

Without wishing to be bound by theory, the symptoms of NS are thought to be caused at least in part by excessive desquamation resulting from dysregulated protease activity in the skin, in particular the protease activity of KLKs. KLK5 may regulate much of this activity, as it is able to self-activate, and also is capable of activating KLK7 and KLK14. Dysregulated KLK5 and/or KLK7 serine protease activity leads to premature detachment of the stratum corneum, the outer skin layer. Loss of stratum corneum can be life-threatening to neonates due to systemic sepsis or severe dehydration due to the compromised epithelial barrier, and it can lead to chronic problems for patients, including allergy, infection, and inflammation.

Spherical nucleic acids (SNAs) are nanoparticle structures that were originally developed with oligonucleotides specifically designed to regulate specific nucleic acid interactions (Mirkin, et al. Nature (1996) 382:607-9). The use of SNAs show many benefits including, but not limited to, rapid cellular uptake, endosomal delivery, and multivalent binding (Radovic-Moreno, et al. PNAS (2015) 112(13):3892-7). Topically applied SNA have shown promising results in reducing gene expression and subsequently T-cell production that could otherwise manifest to psoriasis, a skin disease with autoimmune nature (Nemati, et al. J Control Release (2017) 268:259-68).

Disclosed herein are compositions, such as oligonucleotides (e.g., synthetic oligonucleotides), spherical nucleic acids (SNAs), and methods for decreasing expression of kallikrein-related peptidase (KLK) genes (e.g., KLK5 and KLK7). As disclosed herein, it has been discovered that oligonucleotides (e.g., synthetic oligonucleotides), SNAs, and compositions decrease KLK gene expression and/or KLK protein to levels that promote either partially or completely one or more beneficial phenotype(s) for the treatment, amelioration and/or elimination of one or more, or all, characteristics, conditions, and/or symptoms associated with skin diseases (e.g., chronic skin disease) that can be treated by decreasing KLK5 expression, KLK7 expression or both KLK5 and KLK7 expression.

In some embodiments, the skin disease is an inflammatory skin disease. In some embodiments, the skin disease is a chronic inflammatory skin disease. In some embodiments, the skin disease is an acute inflammatory skin disease. In some embodiments, the inflammatory skin disease causes tissue damage. In some embodiments, the skin disease is associated with inflammation or inflammatory response driven by an immune cell. In some embodiments, the skin disease is eczema. In some embodiments, the skin disease is AD. In some embodiments, the skin disease is rosacea. In some embodiments, the skin disease is psoriasis. In some embodiments, the skin disease is seborrheic dermatitis. In some embodiments, the skin disease is allergic contact dermatitis. In some embodiments, the skin disease is NS.

In some embodiments, oligonucleotides (e.g., synthetic oligonucleotides) disclosed herein comprise nucleosides having modifications, such as a methyl modification. In some embodiments, the modification is a 2′-O-methyl modification.

A 2′-O-methyl modification, or 2′-O-methylation, terms which are used interchangeably to refer to a nucleoside modification in which a methyl group is added to the 2′ hydroxyl of the ribose moiety of a nucleoside, producing a methoxy group. Any nucleobase or nucleoside can be modified by 2′-O-methylation. These modifications can influence various properties of oligonucleotides, including structure, stability, and interactions with other molecules.

In some embodiments, an oligonucleotide (e.g., synthetic oligonucleotide) disclosed herein comprises a nucleoside with a modification, such as a 2′-O-methyl-modification. In some embodiments, an oligonucleotide (e.g., synthetic oligonucleotide) comprises two or at least two nucleosides each comprising a 2′-O-methyl modification. In some embodiments, an oligonucleotide (e.g., a synthetic oligonucleotide) disclosed herein comprises three or at least three, four or at least four, five or at least five, six or at least six, seven or at least seven, eight or at least eight, nine or at least nine, 10 or at least 10, 11 or at least 11, 12 or at least 12, 13 or at least 13, 14 or at least 14, 15 or at least 15, 16 or at least 16, 17 or at least 17, 18 or at least 18, 19 or at least 19, 20 or at least 20 or more nucleosides each comprising a 2′-O-methyl modification.

In some embodiments, the nucleosides are consecutive nucleosides comprising two or at least two, three or at least three, four or at least four, five or at least five, six or at least six, seven or at least seven, eight or at least eight, nine or at least nine, 10 or at least 10, 11 or at least 11, 12 or at least 12, 13 or at least 13, 14 or at least 14, 15 or at least 15, 16 or at least 16, 17 or at least 17, 18 or at least 18, 19 or at least 19, 20 or at least 20 or more consecutive nucleosides each comprising a 2′-O-methyl modification. In some embodiments, the nucleosides are not consecutive.

In some embodiments, an oligonucleotide (e.g., a synthetic oligonucleotide) disclosed herein comprises five consecutive nucleosides each comprising a 2′-O-methyl modification. In some embodiments, an oligonucleotide (e.g., a synthetic oligonucleotide) disclosed herein comprises 20 consecutive nucleosides each comprising a 2′-O-methyl modification. In some embodiments, an oligonucleotide (e.g., a synthetic oligonucleotide) disclosed herein comprises ten nucleosides, either consecutive or discontinuous, each comprising a 2′-O-methyl modification. In some embodiments, an oligonucleotide (e.g., a synthetic oligonucleotide) disclosed herein comprises ten nucleosides comprising 2′-O-methyl modifications.

In some embodiments, oligonucleotides (e.g., synthetic oligonucleotides), SNAs, and compositions target nucleic acid segments (e.g., regions) in a KLK gene (e.g., a KLK5 gene and/or a KLK7 gene) and/or a KLK gene product (e.g., a KLK5 gene product and/or a KLK7 gene product), which are transcribed into mRNA (e.g., exons transcribed into KLK5 mRNA and/or exons transcribed into KLK7 mRNA).

In some embodiments, oligonucleotides (e.g., synthetic oligonucleotides), SNAs, and compositions target nucleic acid segments (e.g., regions) in a KLK gene (e.g., a KLK5 gene and/or a KLK7 gene) and/or a KLK gene product (e.g., a KLK5 gene product and/or a KLK7 gene product), which are not transcribed into mRNA (e.g., introns, 5′-untranslated regions or 5′-UTRs, 3′-untranslated regions or 3′-UTRs, etc.).

In some embodiments, an SNA disclosed herein comprises a first synthetic oligonucleotide comprising a nucleic acid sequence complementary or sufficiently complementary to a region of a first KLK gene and/or to a region of a first KLK gene product, and a second synthetic oligonucleotide comprising a nucleic acid sequence complementary or sufficiently complementary to a region of a second KLK gene and/or to a region of a second KLK gene product. In some embodiments, the first KLK gene is a KLK5 gene. In some embodiments, the first KLK gene product is a KLK5 gene product. In some embodiments, the second KLK gene is a KLK7 gene. In some embodiments, the second KLK gene product is a KLK7 gene product. In some embodiments, the first synthetic oligonucleotide comprises a nucleic sequence complementary or sufficiently complementary to two or more different or non-overlapping regions in a first KLK gene.

In some embodiments, the first synthetic oligonucleotide and the second synthetic oligonucleotide each include nucleic acid sequences which are each complementary or sufficiently complementary to different or non-overlapping regions (e.g., a first region, a second region, a third region, a fourth region, a fifth region, etc.) in the first KLK gene and/or first KLK gene product. In some embodiments, the first KLK gene is a KLK5 gene and both the first synthetic oligonucleotide and the second synthetic oligonucleotide each include nucleic acid sequences which are each complementary or sufficiently complementary to different or non-overlapping regions (e.g., a first region, a second region, a third region, a fourth region, a fifth region, etc.) in a KLK5 gene and/or KLK5 gene product. In some embodiments, the first KLK gene is a KLK7 gene and both the first synthetic oligonucleotide and the second synthetic oligonucleotide each include nucleic acid sequences which are each complementary or sufficiently complementary to different or non-overlapping regions (e.g., a first region, a second region, a third region, a fourth region, a fifth region, etc.) in a KLK7 gene and/or KLK7 gene product.

In some embodiments, the first KLK gene and/or first KLK gene product and the second KLK gene and/or second KLK gene product are the same, such that both the first synthetic oligonucleotide and the second synthetic oligonucleotide each include nucleic acid sequences which are each complementary or sufficiently complementary to different or non-overlapping regions (e.g., a first region, a second region, a third region, a fourth region, a fifth region, etc.) in the same KLK gene and/or KLK gene product. In some embodiments, the first KLK gene and/or first KLK gene product and the second KLK gene and/or second KLK gene product are a KLK5 gene and/or KLK5 gene product. In some embodiments, the first KLK gene and/or first KLK gene product and the second KLK gene and/or second KLK gene product are a KLK7 gene and/or KLK7 gene product.

In some embodiments, the first KLK gene and/or first KLK gene product and the second KLK gene and/or second KLK gene product are different, such that the first synthetic oligonucleotide in a SNA disclosed herein includes a nucleic acid sequence which is complementary or sufficiently complementary to a region in a first KLK gene and/or first KLK gene product and the second synthetic oligonucleotide includes a nucleic acid sequence which is complementary or sufficiently complementary to a region in a second KLK gene and/or second KLK gene product. In some embodiments, the first KLK gene and/or first KLK gene product is a KLK5 gene and/or KLK5 gene product and the second KLK gene and/or second KLK gene product is a KLK7 gene and/or KLK7 gene product. In some embodiments, the SNA comprising the first synthetic oligonucleotide and the second synthetic oligonucleotide decrease expression of a KLK gene product (KLK5 mRNA, KLK7 mRNA, etc.). In some embodiments, the SNA comprising the first synthetic oligonucleotide and the second synthetic oligonucleotide decrease KLK protein levels (KLK5 protein, KLK7 protein, etc.).

In some embodiments, an SNA disclosed herein comprises a synthetic oligonucleotide comprising a nucleic acid sequence complementary or sufficiently complementary to a region of a first KLK gene and/or to a region of a first KLK gene product, and a second synthetic oligonucleotide comprising a nucleic acid sequence complementary or sufficiently complementary to a region of a second KLK gene and/or to a region of a second KLK gene product. In some embodiments, the first KLK gene is a KLK5 gene. In some embodiments, the first KLK gene product is a KLK5 gene product. In some embodiments, the second KLK gene is a KLK7 gene. In some embodiments, the second KLK gene product is a KLK7 gene product. In some embodiments, the first synthetic oligonucleotide comprises a nucleic sequence complementary or sufficiently complementary to two or more different or non-overlapping regions in a first KLK gene.

In some embodiments, a composition comprising two or more SNAs is contemplated herein in which a first SNA disclosed herein comprises a first synthetic oligonucleotide comprising a nucleic acid sequence complementary or sufficiently complementary to a region of a first KLK gene and/or to a region of a first KLK gene product; and a second SNA disclosed herein comprises a second synthetic oligonucleotide comprising a nucleic acid sequence complementary or sufficiently complementary to a region of a second KLK gene and/or to a region of a first KLK gene product. In some embodiments, the composition decreases expression of two or more KLK mRNAs (e.g., KLK5 mRNA and KLK7 mRNA) and/or decrease levels of two or more distinct KLK proteins (e.g., KLK5 protein and KLK7 protein).

In some embodiments, a composition comprising two or more SNAs is contemplated herein in which a first SNA disclosed herein comprises a first synthetic oligonucleotide comprising a nucleic acid sequence complementary or sufficiently complementary to a first region of a KLK gene and/or a KLK gene product; and a second SNA disclosed herein comprises a second synthetic oligonucleotide comprising a nucleic acid sequence complementary or sufficiently complementary to a second region of the KLK gene and/or KLK gene product. In some embodiments, the KLK gene and/or KLK gene product is KLK5 and/or a KLK5 gene product and the composition decreases expression of a KLK5 mRNA and/or decrease levels of KLK5 protein. In some embodiments, the KLK gene and/or KLK gene product is KLK7 and/or a KLK7 gene product and the composition decreases expression of a KLK7 mRNA and/or decrease levels of KLK7 protein.

In some embodiments, a synthetic oligonucleotide disclosed herein comprises a nucleic acid sequence complementary to a region within an exon of a KLK gene (e.g., a KLK5 gene or a KLK7 gene) and/or a KLK gene product (e.g., a KLK5 gene product or a KLK7 gene product). In some embodiments, a synthetic oligonucleotide disclosed herein comprises a nucleic acid sequence complementary to or sufficiently complementary to a region within an intron of a KLK gene (e.g., a KLK5 gene or a KLK7 gene) and/or a KLK gene product (e.g., a KLK5 gene product or a KLK7 gene product). In some embodiments, a synthetic oligonucleotide disclosed herein comprises a nucleic acid sequence complementary to or sufficiently complementary to a region within a 3′-untranslated (3′-UTR) region or a 5′-untranslated region (5′-UTR) of a KLK gene (e.g., a KLK5 gene or a KLK7 gene) and/or a KLK gene product (e.g., a KLK5 gene product or a KLK7 gene product).

In some embodiments, a synthetic oligonucleotide, an SNA, or a composition thereof inhibits expression of a KLK gene (e.g., KLK5 or KLK7) by decreasing the stability of a KLK mRNA molecule (e.g., a KLK5 mRNA molecule or a KLK7 mRNA molecule). In some embodiments, a synthetic oligonucleotide, an SNA, or a composition thereof inhibits expression of a KLK gene (e.g., KLK5 or KLK7) by decreasing ribosome binding to a KLK mRNA molecule (e.g., a KLK5 mRNA molecule or a KLK7 mRNA molecule). In some embodiments, a synthetic oligonucleotide, an SNA, or a composition thereof inhibits expression of a KLK gene (e.g., KLK5 or KLK7) by decreasing the processivity of ribosomes along a KLK mRNA molecule (e.g., a KLK5 mRNA molecule or a KLK7 mRNA molecule). In some embodiments, a synthetic oligonucleotide, an SNA, or a composition thereof inhibits expression of a KLK gene (e.g., KLK5 or KLK7) by decreasing the transcription of a KLK mRNA molecule (e.g., a KLK5 mRNA molecule or a KLK7 mRNA molecule) from a KLK gene. In some embodiments, a synthetic oligonucleotide, an SNA, or a composition thereof inhibits expression of a KLK gene (e.g., KLK5 or KLK7) by interfering with splicing of a KLK pre-mRNA molecule (e.g., a KLK5 pre-mRNA molecule or a KLK7 pre-mRNA molecule). In some embodiments, a synthetic oligonucleotide, an SNA, or a composition thereof inhibits expression of a KLK gene (e.g., KLK5 or KLK7) by activating nonsense-mediated decay of a KLK mRNA or pre-mRNA molecule (e.g., a KLK5 pre-mRNA molecule or a KLK7 pre-mRNA molecule). In some embodiments, a synthetic oligonucleotide, an SNA, or a composition thereof inhibits expression of a KLK gene (e.g., KLK5 or KLK7) by activating RNase H degradation of a KLK mRNA or pre-mRNA molecule (e.g., a KLK5 pre-mRNA molecule or a KLK7 pre-mRNA molecule). In some embodiments, a synthetic oligonucleotide, an SNA, or a composition thereof inhibits expression of a KLK gene (e.g., KLK5 or KLK7) by both altering splicing of a pre-mRNA molecule (e.g., a KLK5 pre-mRNA molecule or a KLK7 pre-mRNA molecule) and by decreasing the stability of a KLK mRNA and/or pre-mRNA molecule (e.g., a KLK5 mRNA and/or pre-mRNA molecule or a KLK7 mRNA and/or pre-mRNA molecule).

In some embodiments, an “oligonucleotide” refers to a nucleic acid sequence with nucleotides (e.g., molecules comprising a sugar, such as ribose or deoxyribose) linked to a phosphate or other suitable chemical group and to an exchangeable base. In some embodiments, the base is an organic base. In some embodiments the base is a pyrimidine (e.g., cytosine (C), thymine (T) or uracil (U)) or a purine (e.g., adenine (A) or guanine (G))). In some embodiments, the oligonucleotide is between eight and 100 nucleobases or nucleosides in length.

In some embodiments, the oligonucleotide is a synthetic oligonucleotide. As disclosed herein, a “synthetic oligonucleotide” refers to a non-naturally occurring oligonucleotide. A synthetic oligonucleotide, in some embodiments, refers to a synthetic DNA or synthetic RNA. In some embodiments, a synthetic oligonucleotide is produced through an in vitro transcription reaction (e.g., artificial (non-natural) chemical synthesis, solid phase nucleic acid synthesis or through another method known by one of ordinary skill in the art). In some embodiments, a synthetic oligonucleotide includes a modification at one or both ends of the nucleic acid sequence in the synthetic oligonucleotide. In some embodiments, the synthetic oligonucleotide is produced by nucleic acid synthesis (e.g., in vitro), chemical nucleic acid synthesis, and/or solid phase nucleic acid synthesis, or produced through other methods well known in the art. In some embodiments, one or more bases in the oligonucleotide (e.g., synthetic oligonucleotide) or nucleic acid sequence include a modification.

In some embodiments, an oligonucleotide (e.g., a synthetic oligonucleotide) disclosed herein comprises a nucleic acid sequence that is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleobases or nucleosides in length, or any range or combination thereof. In some embodiments, the nucleic acid sequence of the synthetic oligonucleotide is 10 to 30, 10 to 35, 10 to 40, 10 to 45, 10 to 50, 10 to 60, 10 to 70, 10 to 80, 10 to 90, 10 to 100 or more than 100 nucleobases or nucleosides in length. In some embodiments, an oligonucleotide (e.g., a synthetic oligonucleotide) disclosed herein comprising a nucleic acid sequence that is 10 to 30 nucleobases or nucleosides in length. In some embodiments, an oligonucleotide (e.g., a synthetic oligonucleotide) disclosed herein is 15 to 22 nucleobases or nucleosides in length. In some embodiments, the oligonucleotide (e.g., synthetic oligonucleotide) is 20 nucleobases or nucleosides in length. In some embodiments, the oligonucleotide (e.g., synthetic oligonucleotide) is 17 nucleobases or nucleosides in length.

In some embodiments, the synthetic oligonucleotide comprises, consists essentially of, or consists of a nucleic acid sequence complementary to or sufficiently complementary to a region (e.g. a nucleic acid sequence segment) of at least one of a kallikrein-related peptidase (KLK) gene (e.g., a KLK5 gene and/or a KLK7 gene), a region of a KLK gene (e.g., a region of KLK5 and/or a region of KLK7), a KLK gene product (e.g., a KLK5 gene product and/or a KLK7 gene product), and a region of a KLK gene product (e.g., a region of a KLK5 gene product and/or a region of a KLK7 gene product). In some embodiments, a synthetic oligonucleotide disclosed herein comprises, consists essentially of, or consists of a nucleic acid sequence complementary to or sufficiently complementary to a region (e.g., a nucleic acid sequence segment) of at least one of SEQ ID NO: 7477, SEQ ID NO: 7478, SEQ ID NO: 7575, SEQ ID NO: 7576, SEQ ID NO: 7577, SEQ ID NO: 7578.

In some embodiments, a nucleic acid sequence of an oligonucleotide (e.g., a synthetic oligonucleotide) is at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97%, at least or about 98%, at least or about 99%, or about 100%, or any range or combination thereof, identical to the nucleic acid sequence of an oligonucleotide (e.g., synthetic oligonucleotide) disclosed herein (e.g., any one of SEQ ID NOs: 1-3302; SEQ ID NOs: 3149-5336; SEQ ID NOs: 5376-5380; SEQ ID NOs: 5414-5418; SEQ ID NOs: 5647-7342; SEQ ID NOs: 7442-7574; SEQ ID NOs: 7579-7803; SEQ ID NOs: 7806-7807; SEQ ID NOs: 7809-7847; and/or an oligonucleotide provided in any one of Tables 1-14).

In some embodiments, an oligonucleotide (e.g., a synthetic oligonucleotide) is at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97%, at least or about 98%, at least or about 99%, or about 100%, or any range or combination thereof, identical to /5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834).

In some embodiments, a nucleic acid sequence of an oligonucleotide (e.g., a synthetic oligonucleotide) is at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97%, at least or about 98%, at least or about 99%, or about 100%, or any range or combination thereof, identical to the nucleic acid sequence of SEQ ID NO: 1312.

In some embodiments, an oligonucleotide (e.g., a synthetic oligonucleotide) is at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97%, at least or about 98%, at least or about 99%, or about 100%, or any range or combination thereof, identical to /CholTEG//iSp18//iSp18/lG*lA*lG*G*T*/iMe-dC/*A*G*A*G*/7deazaG/*G*A*A*lA*lG*lG (SEQ ID NO: 7616).

In some embodiments, a nucleic acid sequence of an oligonucleotide (e.g., a synthetic oligonucleotide) is at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97%, at least or about 98%, at least or about 99%, or about 100%, or any range or combination thereof, identical to the nucleic acid sequence of SEQ ID NO: 3998.

In some embodiments, an oligonucleotide (e.g., a synthetic oligonucleotide) is at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 961%, at least or about 97%, at least or about 98%, at least or about 99%, or about 100%, or any range or combination thereof, identical to lG*lA*lG*A*/iMe-dC/*G*/iMe-dC/*Ad*A*/iMe-dC/*/iMe-dC/*/iMe-dC/*/iMe-dC/*/iMe-dC/*G*/iMe-dC/*/iMe-dC/*/5-Me-lC/*lT*lG/iSp18//iSp18//3CholTEG/ (SEQ ID NO: 7836).

In some embodiments, a nucleic acid sequence of an oligonucleotide (e.g., a synthetic oligonucleotide) is at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97%, at least or about 98%, at least or about 99%, or about 100%, or any range or combination thereof, identical to the nucleic acid sequence of SEQ ID NO: 3118.

In some embodiments, an oligonucleotide (e.g., a synthetic oligonucleotide) disclosed herein is any size useful for producing an effect (e.g., promote either partially or completely one or more beneficial phenotype(s) for the treatment, amelioration and/or elimination of one or more characteristics, conditions, and/or symptoms associated with NS, as disclosed herein).

In some embodiments, the oligonucleotide (e.g., synthetic oligonucleotide) is single-stranded. In some embodiments, the oligonucleotide (e.g., synthetic oligonucleotide) is hybridized to a second oligonucleotide (e.g., synthetic oligonucleotide) and forms a double-stranded oligonucleotide. In some embodiments, the oligonucleotide (e.g., synthetic oligonucleotide) is not hybridized to a second oligonucleotide and does not form a double-stranded oligonucleotide.

In some embodiments, an oligonucleotide (e.g., a synthetic oligonucleotide) is a single-stranded oligonucleotide.

In some embodiments, a synthetic oligonucleotide comprises, consists essentially of, or consists of a 5′-wing segment, a 3′-wing segment, and a gap segment.

In some embodiments, an oligonucleotide (e.g., a synthetic oligonucleotide) has a gap segment.

As disclosed herein, a “gap segment” corresponds to two or more linked nucleosides in a synthetic oligonucleotide, which are positioned between the 5′-wing segment and the 3′-wing segment. In some embodiments, an oligonucleotide (e.g., a synthetic oligonucleotide) does not have a gap segment. In some embodiments, a gap segment refers to one or more linked nucleosides located at the center or near the center of an oligonucleotide, such as a synthetic oligonucleotide. In some embodiments, the gap segment consists of two to three, two to four, two to five, two to six, two to seven, two to eight, two to nine, two to 10, two to 20, two to 30, two to 40, two to 50, three to four, three to five, three to six, three to seven, three to eight, three to nine, three to 10, three to 20, three to 30, three to 40, three to 50, four to five, four to six, four to seven, four to eight, four to nine, four to 10, four to 20, four to 30, four to 40, four to 50, five to six, five to seven, five to eight, five to nine, five to 10, five to 20, five to 30, five to 40, five to 50, six to seven, six to eight, six to nine, six to 10, six to 20, six to 30, six to 40, six to 50, seven to eight, seven to nine, seven to 10, seven to 20, seven to 30, seven to 40, seven to 50, eight to nine, eight to 10, eight to 20, eight to 30, eight to 40, eight to 50, 10 to 20, 10 to 30, 10 to 40, 10 to 50, 20 to 30, 20 to 40, 20 to 50, 30 to 40, 40 to 50, or 50 linked nucleosides, or any range or combination thereof.

In some embodiments, the gap segment comprises a nucleoside having a 2′-O-methyl modification. In some embodiments, the gap segment comprises two to 10 nucleosides having 2′-O-methyl modifications. In some embodiments, the gap segment does not comprise a nucleoside having a 2′-O-methyl modification. In some embodiments, the gap segment comprises a phosphorothioate internucleoside linkage. In some embodiments, the gap segment comprises two to 10 phosphorothioate internucleoside linkages. In some embodiments, the gap segment comprises all phosphorothioate internucleoside linkages. In some embodiments, the gap segment does not comprise phosphorothioate internucleoside linkages.

In some embodiments, the gap segment comprises a phosphodiester internucleoside linkage. In some embodiments, the gap segment comprises two to 10 phosphodiester internucleoside linkages. In some embodiments, the gap segment comprises all phosphodiester internucleoside linkages. In some embodiments, the gap segment does not comprise phosphodiester internucleoside linkages.

As disclosed herein, a “5′-wing segment” corresponds to two or more linked nucleosides positioned at the 5′-end of a nucleic acid sequence in a synthetic oligonucleotide and corresponding to nucleosides positioned before the first nucleoside at the 5′-end of a gap segment. As disclosed herein, a “3′-wing segment” corresponds to two or more linked nucleosides positioned after the last nucleic acid at the 3′ end of the gap segment and including the last nucleic acid at the 3′ end of the synthetic oligonucleotide. In some embodiments, at least one nucleoside of the 5′-wing segment and/or at least one nucleoside of the 3′-wing segment comprises a modification.

In some embodiments, the modification is a 2′-O-methyl modification. In some embodiments, the nucleosides in the synthetic oligonucleotide are modified with one or more other modifications disclosed herein. In some embodiments, the internucleoside linkages within the gap segment and the linkages connecting the gap segment to the 3′-wing segment and/or the 5′-wing segment are all phosphorothioate linkages (*). In some embodiments, the internucleoside linkages connecting the rest of the nucleosides of both the 5′ and 3′-wing segments are phosphodiester linkages. In some embodiments, the internucleoside linkages connecting the rest of the nucleosides of both the 5′ and 3′-wing segments are phosphorothioate linkages (*). In some embodiments, all internucleoside linkages connecting the nucleosides of the 5′-wing segment, the gap segment, and the 3′-wing segment are phosphorothioate linkages (*).

As disclosed herein, in some embodiments, a “nucleic acid” refers to multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). As used herein, the term nucleic acid refers to polyribonucleotides as well as polydeoxyribonucleotides. The term nucleic acid shall also include polynucleosides (i.e., a polynucleotide minus the phosphate) and any other organic base containing polymer. Non-limiting examples of nucleic acids include chromosomes, genomic loci, genes or gene segments that encode polynucleotides or polypeptides, coding sequences, non-coding sequences (e.g., intron, 5′-UTR, or 3′-UTR) of a gene, pre-mRNA, mRNA, etc.

In some embodiments, the nucleic acid sequence and/or oligonucleotide (e.g., a synthetic oligonucleotide) includes a substitution and/or modification. In some embodiments, the substitution and/or modification is in one or more bases and/or sugars. For example, in some embodiments a nucleic acid sequence and/or oligonucleotide (e.g., a synthetic oligonucleotide) includes nucleic acids having backbone sugars that are covalently attached to low molecular weight organic groups other than a hydroxyl group at the 2′ position and other than a phosphate group or hydroxyl group at the 5′ position. Thus, in some embodiments, a substituted or modified nucleic acid sequence and/or oligonucleotide (e.g., a synthetic oligonucleotide) includes a 2′-O-alkylated ribose group. In some embodiments, a modified nucleic acid sequence and/or oligonucleotide (e.g., a synthetic oligonucleotide) includes sugars such as hexose, 2′-F hexose, 2′-amino ribose, constrained ethyl (CEt), locked nucleic acid (LNA, also known as bridged nucleic acid (BNA)), arabinose or 2′-fluoroarabinose instead of ribose. Thus, in some embodiments, a nucleic acid sequence and/or oligonucleotide (e.g., a synthetic oligonucleotide) is heterogeneous in backbone composition thereby containing any possible combination of polymer units linked together such as peptide-nucleic acids (which have an amino acid backbone with nucleic acid bases).

In some embodiments, the nucleic acid sequence and/or oligonucleotide (e.g., synthetic oligonucleotide) includes one or more 2′-O-methoxyethyl (2′-MOE) modifications or modified nucleosides. In some embodiments, the nucleic acid sequence and/or oligonucleotide (e.g., synthetic oligonucleotide) includes one or more LNA modifications or modified nucleosides. In some embodiments, the nucleic acid sequence and/or oligonucleotide (e.g., synthetic oligonucleotide) includes a 2′-MOE modified nucleoside and an LNA modification or modified nucleoside.

In some embodiments, a nucleic acid sequence and/or oligonucleotide (e.g., synthetic oligonucleotide) disclosed herein includes at least one LNA modification or modified nucleoside. An LNA modification or modified nucleoside is a modified RNA nucleoside in which the ribose moiety is modified with an additional bond connecting the 2′ oxygen and the 4′ carbon.

Without wishing to be bound by theory, LNA modifications, such as in LNA modified nucleosides, enhance base stacking and backbone organization, and significantly increase the hybridization properties of oligonucleotides. In some embodiments, the melting temperature of oligonucleotides comprising an LNA modification(s) or modified nucleoside(s) can be increased relative to an unmodified oligonucleotide having the same nucleic acid sequence.

In some embodiments, the LNA modification or modified nucleoside is, comprises, consists essentially of, or consists of (2′-O, 4′-C methylene)-Adenosine. In some embodiments, the LNA modification or modified nucleoside is, comprises, consists essentially of, or consists of 5-methyl-(2′-O, 4′-C methylene)-Cytidine. In some embodiments, the LNA modification or modified nucleoside is, comprises, consists essentially of, or consists of (2′-O, 4′-C methylene)-Cytidine. In some embodiments, the LNA modification or modified nucleoside is, comprises, consists essentially of, or consists of (2′-O, 4′-C methylene)-Guanosine. In some embodiments, the LNA modification or modified nucleoside is, comprises, consists essentially of, or consists of 5-methyl-(2′-O, 4′-C methylene)-Uridine. In some embodiments, the nucleic acid sequence and/or oligonucleotide (e.g., synthetic oligonucleotide) includes two or more LNA modification or modified nucleosides, each of which, in some embodiments, comprises, consists of, or consists essentially of an LNA modification or modified nucleoside disclosed herein.

In some embodiments, a nucleic acid sequence and/or oligonucleotide (e.g., a synthetic oligonucleotide) is DNA, RNA, PNA, CEt, LNA, ENA or hybrids including any chemical or natural modification thereof. Chemical and natural modifications are well known in the art. Non-limiting examples of modifications include modifications designed to increase binding to a target strand (i.e., increase their melting temperatures), to assist in identification of the oligonucleotide or an oligonucleotide-target complex, to increase cell penetration, to stabilize against nucleases and other enzymes that degrade or interfere with the structure or activity of the oligonucleotides, to provide a mode of disruption (a terminating event) once sequence-specifically bound to a target, and to improve the pharmacokinetic properties of the oligonucleotide.

Modifications include, but are not limited to, for example, (a) end modifications, e.g., 5′ end modifications (phosphorylation, dephosphorylation, conjugation, inverted linkages, etc.) and 3′ end modifications (conjugation, DNA nucleotides, inverted linkages, etc.); (b) base modifications, e.g., replacement with modified bases, stabilizing bases, destabilizing bases, bases that base pair with an expanded repertoire of partners, or conjugated bases; (c) sugar modifications (e.g., at the 2′ position or 4′ position) or replacement of the sugar; as well as (d) internucleoside linkage modifications, including modification or replacement of the phosphodiester linkages. An oligonucleotide (e.g., a synthetic oligonucleotide disclosed herein) can comprise one or more of any of these modifications, or a combination thereof.

In some embodiments, an oligonucleotide (e.g., a synthetic oligonucleotide) disclosed herein comprises at least one modification or modified nucleoside (e.g., 2′-MOE modification or modified nucleoside, an LNA modification or modified nucleoside, or any other modification or modified nucleoside disclosed herein). In some embodiments, the oligonucleotide (e.g., a synthetic oligonucleotide) comprises at least two modifications or modified nucleosides. In some embodiments, the at least two modifications or modified nucleosides comprise an LNA modification or modified nucleoside and a modification or modified nucleoside disclosed herein.

In some embodiments, the at least two modifications or modified nucleosides comprise a 2′-MOE modification or modified nucleoside and a modification or modified nucleoside disclosed herein. In some embodiments, the at least two modifications or modified nucleosides comprise a 2′-MOE modification or modified nucleoside and an LNA modification or modified nucleoside. In some embodiments, the oligonucleotide (e.g., a synthetic oligonucleotide) comprises two, three, four, five, six, seven, eight, nine, 10, 11, 12, 13, 14, 15, 16, 17, 18, 19, 20 or more than 20 modifications or modified nucleosides (e.g., 2′-MOE modification or modified nucleoside, an LNA modification or modified nucleoside, or any other modification or modified nucleoside disclosed herein).

In some embodiments, a nucleic acid sequence and/or oligonucleotide (e.g., synthetic oligonucleotide) disclosed herein comprises an end modification. In some embodiments, 5′- and/or 3′-end modifications involve incorporation or addition of non-native or non-natural components to the 5′- and/or 3′-end of a nucleic acid or oligonucleotide (e.g., synthetic oligonucleotide). Such modifications may improve physicochemical properties, stability, resistance to nuclease degradation, etc. End modifications may include the addition of amino modifiers (e.g., 5′-DMS(O)MT-amino modifier C6, 5′-amino modifier C3-TFA, 5′-amino modifier C12, 5′-amino modifier C6-TFA, 5′-amino-dT, 5′-amino modifier-5, and 3′-amino modifier C7-CPG); thiol modifiers (e.g., 5′-thiol-modifier C6 S-S and 3′-thiol-modifier C6 S-S); 3′-glyceryl modification; binding modifiers (e.g., 5′-biotin, biotin-dT, biotin-TEG, 3′-biotin-TEG-CPG, digoxigenin, and 2,4-dinitrophenol-TEG); spacers (e.g., spacer 9, spacer 12, spacer 18, spacer C3, 3′-spacer-C3-CPG); nucleoside/nucleotide analogs (e.g., 3′-deoxynucleoside-CPG, 2′,3′-dideoxycytidine, halogenated bases, 2′-deoxypseudouridine, 5,6-dihydro-dT, 5,6-dihydro-dU, 5-OH-dC, 5-OH-dU, 8-oxo-dA, 8-oxo-dG, thymidine glycol, dUracil, 2′-deoxynebularine, derivative K, derivative P, inosine, 5-nitroindole, 3-nitropyrrole, 2,6-diaminopurine, 5-Me-dC, 2-aminopurine, etheno-dA, N6-Me-dA, 06-Me-dG, 04-Me-dT, dSpacer, 5′-O-MedT, 7-deaza-dA, 7-deaza-dG, 7-deaza-dX, 7-deaza-8-aza-dA, and puromycin), intercalators (e.g., psoralen C2, and psoralen C6); cholesterol moieties (e.g., cholesteryl-TEG and 3′-cholesteryl-TEG-CPG); methyl RNA nucleotides (e.g., 2′-OMe-A, 2′-OMe-C, 2′-OMe-G and 2′-OMe-U); and/or thiophosphates to the 5′-end and/or the 3′-end of a nucleic acid or oligonucleotide.

In some embodiments, a nucleic acid sequence and/or oligonucleotide (e.g., synthetic oligonucleotide) disclosed herein comprises a base modification. In some embodiments, a base modification involves replacement of a “natural” or “native” nucleobase of a nucleic acid sequence and/or oligonucleotide (e.g., a synthetic oligonucleotide) with a “non-natural” or “non-native” substituent, or involves chemical modification of a native nucleobase. Non-limiting examples of base modifications include methylation, hydroxymethylation, alkylation, methoxyethyl modifications, and substitutions with heterocyclic, stabilizing, destabilizing, promiscuous, or conjugated base moieties. “Natural” nucleobases include the purine bases adenine and guanine, and the pyrimidine bases thymine, cytosine and uracil. “Non-native” or “non-natural” substituents include 5-methyl-cytosine (5-Me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (e.g., 5-bromo), 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Base modifications also include replacement of the native purine or pyrimidine base with other heterocycles, such as 7-deaza-adenine, 7-deazaguanosine, 2-aminopyridine and 2-pyridone, 5-substituted pyrimidines, 6-azapyrimidines and N-2, N-6 and 0-6 substituted purines, including 2-aminopropyladenine, 5-propynyluracil and 5-propynylcytosine. Base modifications can improve various oligonucleotide properties, including stability (e.g., nuclease resistance, thermostability, chemical stability, biological stability, etc.), target hybridization, biocompatibility (e.g., reduced hepatotoxicity, nephrotoxicity, immune stimulation, etc.), mismatch discrimination, water solubility, etc.

In some embodiments, a nucleic acid sequence and/or oligonucleotide (e.g., synthetic oligonucleotide) disclosed herein comprises one or more (e.g., two or more, three or more, etc.) modified nucleosides (e.g., modified nucleosides with a base modification(s) and/or modified nucleosides with a sugar modification(s)). In some embodiments, the modification or modified nucleoside is a 2′-O-methyl (2′-O-Me) modification or 2′-O-Me modified nucleoside, a 2′-O-methoxyethyl (2′-MOE or MOE) modification or 2′-MOE modified nucleoside, a 2′-O-methoxyethoxy-5-methyl (5-Me-MOE) modification or 5-Me-MOE modified nucleoside, an LNA modification or LNA modified nucleoside, a 5-methyl (5-Me or iMe) modification or 5-Me or iMe modified nucleoside (e.g., 5-methylcytidine or 5-methyluridine), a 5-methyl LNA modification or 5-methyl LNA modified nucleoside, a 7-deaza modification or 7-deaza modified nucleoside, or a 7-deaza-2′-O-methyl (7deazaOM) modification or 7deazaOM modified nucleoside.

In some embodiments, a nucleic acid sequence and/or oligonucleotide (e.g., synthetic oligonucleotide) disclosed herein comprises an LNA modification or LNA modified nucleoside, and a 5-methyl modification or 5-methyl modified nucleoside. In some embodiments, a nucleic acid sequence and/or oligonucleotide (e.g., synthetic oligonucleotide) disclosed herein comprises an LNA modification or LNA modified nucleoside, a 5-methyl modification or 5-methyl modified nucleoside, and a 7-deaza modification or 7-deaza modified nucleoside. In some embodiments, a nucleic acid sequence and/or oligonucleotide (e.g., synthetic oligonucleotide) disclosed herein further comprises one or more modified internucleoside linkages, such as phosphorothioate internucleoside linkage(s). As a non-limiting example, the nucleic acid sequence and/or oligonucleotide (e.g., synthetic oligonucleotide) comprises one or more LNA modified nucleosides, one or more 5-methyl modified nucleosides, one or more 7-deaza modified nucleosides, and one or more phosphorothioate internucleoside linkages.

In some embodiments, a modified nucleoside is a 2′-O-methyl adenosine (mA), a 2′-O-methyl cytidine (mC), a 2′-O-methyl guanosine (mG), a 2′-O-methyl uridine (mU), a deoxyadenosine (A, dA), a deoxycytidine (C, dC), a deoxyguanosine (G, dG), a deoxythymidine (T, dT), a 2′-O-methoxyethoxy adenosine (moeA, 2′-MOE-A), a 2′-O-methoxyethoxy-5-methyl cytidine (5-Me-MOE-C), a 2′-O-Methoxyethoxy Guanosine (moeG, 2′-MOE-G), a 2′-O-Methoxyethoxy-5-Methyl Uridine (moeT, 2′-MOE-T), an LNA Adenosine (lA), an LNA 5-Methyl Cytidine (5-Me-lC), an LNA Guanosine (lG), an LNA Thymidine (lT), a 5-Methyl deoxy Cytidine (iMe-dC), a 7-Deaza deoxy Guanosine (7deazaG), or a 7-deaza-2′-O-Methyl Guanosine (7deazaOMG).

In some embodiments, a nucleic acid sequence and/or oligonucleotide (e.g., synthetic oligonucleotide) disclosed herein comprises a sugar modification. In some embodiments, a sugar modification involves replacement of a “natural” or “native” sugar ring of a nucleoside of a nucleic acid sequence and/or oligonucleotide (e.g., a synthetic oligonucleotide) with a “non-natural” or “non-native” substituent, or involves chemical modification of a native sugar ring. Sugar ring substituent groups include OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C2 to C₁₀ alkenyl and alkynyl. These include O[(CH₂)ₙO]ₘCH₃, O(CH₂)ₙOCH₃, O(CH₂)ₙNH₂, O(CH₂)ₙCH₃, O(CH₂)₂ONH₂, and O(CH₂)ₙON[(CH₂)ₙCH₃]₂, where n and m are from 1 to about 10. Other substituent groups include C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide or a group for improving the pharmacodynamic properties of an oligonucleotide, and other substituents having similar properties. In some embodiments, a sugar modification includes a 2′-O-methoxyethoxy (2′-O—CH₂CH₂OCH₃, also known as 2′-O-(2-methoxyethyl) or 2′-MOE; i.e., an alkoxyalkoxy group), a 2′-dimethylaminooxyethoxy (i.e., a O(CH₂)₂ON(CH₃)₂ group, also known as 2′-DMAOE), or 2′-dimethylaminoethoxyethoxy (also known as 2′-O-dimethyl-amino-ethoxy-ethyl or 2′-DMAEOE; i.e., 2′-O—CH₂—O—CH₂—N(CH₃)₂). In some embodiments, a sugar modification includes an LNA modification, a 2′-O-Me modification, or a 2′-MOE modification.

In some embodiments, a nucleic acid sequence and/or oligonucleotide (e.g., synthetic oligonucleotide) disclosed herein comprises an internucleoside linkage modification. In some embodiments, an internucleoside linkage modification involves replacement of a “natural” or “native” internucleoside linkage of a nucleic acid sequence and/or oligonucleotide (e.g., a synthetic oligonucleotide) with a “non-natural” or “non-native” substituent, or involves chemical modification of a native internucleoside linkage. In some embodiments, an internucleoside linkage modification may comprise replacement of an oxygen of the phosphate group in the 3′,5′-phosphodiester bond with a substituent atom or a substituent group, or may comprise replacement of the 3′-5′-phosphodiester bond or both the 3′,5′-phosphodiester bond and the sugar moiety to facilitate linkage of one nucleobase of a nucleic acid molecule to the next. Non-limiting examples of modified internucleoside linkages include phosphorothioate, phosphorodithioate, N3′ phosphoramidate, boranophosphate, 2′,5′-phosphodiester, phosphonoacetate (PACE), methylphosphonate, morpholino, amide, and peptide nucleic acid linkages. In some embodiments, an internucleoside linkage modification comprised in a nucleic acid sequence and/or oligonucleotide (e.g., synthetic oligonucleotide) disclosed herein is a phosphorothioate internucleoside linkage modification.

In some embodiments, a nucleic acid sequence and/or oligonucleotide (e.g., a synthetic oligonucleotide) comprises a modified backbone. In some embodiments, the modified backbone comprises modified internucleoside linkages. In some embodiments, the modified backbone comprises one or more phosphorothioate internucleoside linkages. In some embodiments, all of the internucleoside linkages of the oligonucleotide are phosphorothioate internucleoside linkages. In some embodiments, modified internucleoside linkages (e.g., linkages within a modified backbone) that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones, amide backbones; and others having mixed N, O, S and CH2 component parts.

Non-limiting examples of modified internucleoside linkages include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Substituted sugar moieties include, but are not limited to one of the following at the 2′ position: H (deoxyribose); OH (ribose); F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10 alkyl or C2 to C10 alkenyl and alkynyl.

In some embodiments, a synthetic oligonucleotide includes, for example, at least one nucleotide or nucleoside modified at the 2′ position of the sugar. In some embodiments, the nucleoside modification is a 2′-O-alkyl, 2′-O-alkyl-O-alkyl or 2′-fluoro-modified nucleotide or an end cap. In some embodiments, nucleoside modifications include 2′-fluoro, 2′-amino and 2′ O-methyl modifications on the ribose of pyrimidines, abasic residues or an inverted base at the 3′ end of the oligonucleotide. In some embodiments, a synthetic oligonucleotide includes a single modified nucleoside. In some embodiments, a synthetic oligonucleotide includes at least two modified nucleosides, at least 3, at least 4, at least 5, at least 6, at least 7, at least 8, at least 9, at least 10, at least 15, at least 20 or more nucleosides, up to the entire length of the oligonucleotide (e.g., synthetic oligonucleotide). In some embodiments, each nucleoside of the synthetic oligonucleotide is a modified nucleoside.

In some embodiments, nucleosides or nucleobases include the natural purine bases adenine (A) and guanine (G), and the pyrimidine bases thymine (T), cytosine (C) and uracil (U). In some embodiments, modified nucleosides include other synthetic and natural nucleobases such as inosine, xanthine, hypoxanthine, nubularine, isoguanisine, tubercidine, 2-(halo)adenine, 2-(alkyl)adenine, 2-(propyl)adenine, 2 (amino)adenine, 2-(aminoalkyll)adenine, 2 (aminopropyl)adenine, 2 (methylthio) N6 (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl) adenine, 8-(thiol)adenine, N6-(isopentyl)adenine, N6 (methyl)adenine, N6, N6 (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, 5 (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, 5 (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, 5 (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 aminocarbonylethylenyl)-2,4-(dithio)pseudouracil, 1 (arninoalkylarninocarbonylethylenyl)-pseudouracil, 1 (arninoalkylarnino-carbonylethylenyl)-2(thio)-pseudouracil, 1(arninoalkylarninocarbonylethylenyl)-4 (thio)pseudouracil, 1 (arninoalkylarninocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(arninoalkylhydroxy)-1,3-(diaza)-2-(oxo)-phenoxazin-1-yl, 7-(arninoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, diiluorotolyl, 4-(iluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino) purine, 5 substituted pyrimidines, N2-substituted purines, N6-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof.

In some embodiments, a synthetic oligonucleotide is a chimeric oligonucleotide. Chimeric oligonucleotides may be formed as composite structures of two or more oligonucleotides, modified oligonucleotides, and/or oligonucleotide mimetics. Such compounds have also been referred to in the art as hybrids or mixed backbone or chimeric or gapmers. In particular a gapmer is an oligonucleotide that has at least three discrete portions, two of which are similar i.e. include one or more backbone modifications or include one or more nucleoside modifications, and surround a region that is distinct, i.e., does not include backbone modifications or does not include nucleoside modifications.

In some embodiments, a “plurality of synthetic oligonucleotides” refers to two or more synthetic oligonucleotides. In some embodiments, the plurality of two or more synthetic oligonucleotides refers to a plurality of first synthetic oligonucleotides that form a first population of synthetic oligonucleotides. In some embodiments, the plurality of two or more synthetic oligonucleotides refers to a plurality of second synthetic oligonucleotides that form a second population of synthetic oligonucleotides.

In some embodiments, the plurality of synthetic oligonucleotides (e.g., in a first population of synthetic oligonucleotides, in a second population of synthetic oligonucleotides, etc.) comprises two, three, four, five, six, seven, eight, nine, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 600, 700, 800, 900, 1000, 1500, 2000, 2500, or more than 2,500 synthetic oligonucleotides, or any range or combination thereof. In some embodiments, the plurality of synthetic oligonucleotides (e.g., in a first population of synthetic oligonucleotides, in a second population of synthetic oligonucleotides, etc.) comprises from two to 2,000 synthetic oligonucleotides.

In some embodiments, the plurality of synthetic oligonucleotides (e.g., in a first population of synthetic oligonucleotides, in a second population of synthetic oligonucleotides, etc.) comprises at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, at least 150, at least 200, at least 250, at least 300, at least 350, at least 400, at least 450, at least 500, at least 600, at least 700, at least 800, at least 900, at least 1000, at least 1500, at least 2000, or at least 2500, or any range or combination thereof. In some embodiments, the two or more synthetic oligonucleotides in the plurality of synthetic oligonucleotides (e.g., in a first population of synthetic oligonucleotides, in a second population of synthetic oligonucleotides, etc.) each comprise nucleic acid sequences, which are not the same, such as any of the nucleic acid sequences disclosed herein.

As used herein, the phrase “the first synthetic oligonucleotide and second synthetic oligonucleotide are not the same” or equivalent phrases are used to refer to two synthetic oligonucleotides which differ in at least one of nucleic acid sequence, structure, length, and modification (e.g., base and/or backbone modifications, etc.). In some embodiments, two or more synthetic oligonucleotides which are not the same have different nucleic acid sequences (e.g., the two or more synthetic oligonucleotides have one or more nucleotides which are not the same when the two or more synthetic oligonucleotides are aligned in the same direction, such as in a 5′ to 3′ direction). In some embodiments, two or more oligonucleotides which are not the same have sequences which have different sequences that differ in at least one or more different nucleotides or nucleosides. In some embodiments, the first synthetic oligonucleotide and second synthetic oligonucleotide that are not the same have one or more nucleotide substitutions at one or more corresponding positions within the oligonucleotide relative to one another. In some embodiments, two or more oligonucleotides which are not the same have one or more different internucleoside linkages at one or more positions within the oligonucleotide. In some embodiments, two or more synthetic oligonucleotides which are not the same comprise, consist of, or consist essentially of sequences with different SEQ ID NOs, according to any of the SEQ ID NOs disclosed herein.

For instance, in a non-limiting example, two or more synthetic oligonucleotides which are not the same refers to one or a first synthetic oligonucleotide having one or a first SEQ ID NO disclosed herein and a second synthetic oligonucleotide having one or a second SEQ ID NO disclosed herein, wherein the first SEQ ID NO and the second SEQ ID NO are not the same SEQ ID NO. In some embodiments, two or more nucleic acid sequences which are not the same comprise or consist of or consist essentially of sequences with different SEQ ID NOs, according to any of the SEQ ID NOs disclosed herein.

As disclosed herein, an oligonucleotide (e.g., a synthetic oligonucleotide) is “complementary” or “sufficiently complementary” to a nucleic acid (e.g. a region of a KLK gene disclosed herein) when hybridization can occur in an antiparallel configuration between the oligonucleotide (e.g., a synthetic oligonucleotide) and the nucleic acid. A double-stranded nucleic acid or oligonucleotide (e.g. synthetic oligonucleotide) can be “complementary” or “sufficiently complementary” to another nucleic acid or oligonucleotide (e.g., a synthetic oligonucleotide) if hybridization can occur between one of the strands of the first nucleic acid or oligonucleotide and the second nucleic acid or oligonucleotide (e.g., a synthetic oligonucleotide). Complementarity (e.g., the degree to which one polynucleotide is complementary with another) is quantifiable in terms of the proportion of bases in opposing strands that are expected to form hydrogen bonds with each other, according to generally accepted base pairing (e.g., Watson-Crick base pairing) rules. An oligonucleotide (e.g., a synthetic oligonucleotide) may be complementary to or sufficiently complementary to a region of a nucleic acid even if the two base sequences are not 100% complementary, as long as the duplex structure formed between has the desired stability.

In some embodiments, two nucleic acids are considered complementary to or sufficiently complementary to one another when their nucleic acid sequences are able to form base pairing interactions (e.g., Watson-Crick base pairing interactions) between at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97%, at least or about 98%, at least or about 99%, or about 100% of their nucleotides, or any range or combination thereof. In some embodiments, two nucleic acid sequences are considered complementary to or sufficiently complementary to one another when their nucleic acid sequences form base pairing interactions (e.g., Watson-Crick base pairing interactions) between at least or about 45% of their nucleotides.

In some embodiments, two nucleic acid sequences are considered complementary to or sufficiently complementary to one another when their nucleic acid sequences form base pairing interactions between at least or about 50% of their nucleotides.

In some embodiments, two nucleic acid sequences are considered complementary to or sufficiently complementary to one another when their nucleic acid sequences form base pairing interactions between at least or about 55% of their nucleotides.

In some embodiments, two nucleic acid sequences are considered complementary to or sufficiently complementary to one another when their nucleic acid sequences form base pairing interactions between at least or about 60% of their nucleotides.

In some embodiments, two nucleic acid sequences are considered complementary to or sufficiently complementary to one another when their nucleic acid sequences form base pairing interactions between at least or about 65% of their nucleotides.

In some embodiments, two nucleic acid sequences are considered complementary to or sufficiently complementary to one another when their nucleic acid sequences form base pairing interactions between at least or about 70% of their nucleotides.

In some embodiments, two nucleic acid sequences are considered complementary to or sufficiently complementary to one another when their nucleic acid sequences form base pairing interactions between at least or about 75% of their nucleotides.

In some embodiments, two nucleic acid sequences are considered complementary to or sufficiently complementary to one another when their nucleic acid sequences form base pairing interactions between at least or about 80% of their nucleotides.

In some embodiments, two nucleic acid sequences are considered complementary to or sufficiently complementary to one another when their nucleic acid sequences form base pairing interactions between at least or about 85% of their nucleotides.

In some embodiments, two nucleic acid sequences are considered complementary to or sufficiently complementary to one another when their nucleic acid sequences form base pairing interactions between at least or about 90% of their nucleotides.

In some embodiments, two nucleic acid sequences are considered complementary to or sufficiently complementary to one another when their nucleic acid sequences form base pairing interactions between at least or about 92.5% of their nucleotides.

In some embodiments, two nucleic acid sequences are considered complementary to or sufficiently complementary to one another when their nucleic acid sequences form base pairing interactions between at least or about 95% of their nucleotides.

In some embodiments, two nucleic acid sequences are considered complementary to or sufficiently complementary to one another when their nucleic acid sequences form base pairing interactions between at least or about 96% of their nucleotides.

In some embodiments, two nucleic acid sequences are considered complementary to or sufficiently complementary to one another when their nucleic acid sequences form base pairing interactions between at least or about 97% of their nucleotides.

In some embodiments, two nucleic acid sequences are considered complementary to or sufficiently complementary to one another when their nucleic acid sequences form base pairing interactions between at least or about 98% of their nucleotides.

In some embodiments, two nucleic acid sequences are considered complementary to or sufficiently complementary to one another when their nucleic acid sequences form base pairing interactions between at least or about 99% of their nucleotides.

In some embodiments, two nucleic acid sequences are considered complementary to or sufficiently complementary to one another when their nucleic acid sequences form base pairing interactions between at least or about 100% of their nucleotides.

In a non-limiting example, two nucleic acid sequences that are each 20 nucleosides in length are considered 80% complementary if 16 of their respective 20 nucleosides are able to form base-pairing interactions. Two nucleic acid sequences that are of different lengths may also be complementary. For example, a nucleic acid sequence that is 20 nucleosides in length may be complementary to or sufficiently complementary to a nucleic acid sequence that is longer if their sequences are able to form base pairing interactions (e.g., Watson-Crick base pairing interactions) between a sufficient number of nucleotides of the 20 nucleoside length nucleic acid sequence and a sufficient number of nucleotides of the longer nucleic acid sequence. A nucleic acid sequence that is 20 nucleosides in length is considered 100% complementary to or sufficiently complementary to a longer nucleic acid sequence if all 20 of its nucleosides are able to form base-pairing interactions with 20 sequential nucleosides of the longer nucleic acid sequence. Similarly, a nucleic acid sequence that is 20 nucleosides in length is considered 80% complementary to or sufficiently complementary to a longer nucleic acid sequence if 16 of its 20 nucleosides are able to form base-pairing interactions with 16 nucleosides of the longer nucleic acid sequence in a complementary sequence alignment.

In some embodiments, a synthetic oligonucleotide comprises a molecular species. In some embodiments, the molecular species anchors the synthetic oligonucleotide to the core (e.g., liposome core) of an SNA disclosed herein. A molecular species may be attached at various positions of a synthetic oligonucleotide (e.g., the 3′-end or 5′-end of a nucleic acid sequence of a synthetic oligonucleotide disclosed herein). In some embodiments, the molecular species enhances the stability of the synthetic oligonucleotide against 3′- or 5′-exonucleases. In some embodiments, a molecular species is attached to or associated with an internal nucleotide or a nucleotide on a branch of a synthetic oligonucleotide. In some embodiments, a molecular species is attached to a 2-position of a nucleoside of a synthetic oligonucleotide. In some embodiments, a molecular species is linked to the heterocyclic base of a nucleoside of a synthetic oligonucleotide.

In some embodiments, the molecular species is modified. In some embodiments, the molecular species comprises multiple moieties. In some embodiments, the molecular species is, comprises, consists essentially of, or consists of a lipophilic (i.e., hydrophobic) moiety. In some embodiments, the molecular species is, comprises, consists essentially of, or consists of a lipophilic (i.e., hydrophobic) moiety and a hydrophilic moiety. In some embodiments, the molecular species comprises multiple lipophilic (i.e., hydrophobic) moieties and/or multiple hydrophilic moieties. In some embodiments, the molecular species is, comprises, consists essentially of, or consists of a hydrophobic moiety, such as a cholesterol or a cholesteryl ester.

In some embodiments, the molecular species is, comprises, consists essentially of, or consists of a cholesterol and an additional moiety. In some embodiments, the molecular species is, comprises, consists essentially of, or consists of a cholesterol and a hydrophilic moiety. In some embodiments, the molecular species comprises a cholesterol and a triethylene glycol (TEG). In some embodiments, the molecular species comprises one or more cholesterols and one or more TEGs. In some embodiments, the molecular species comprises a cholesterol and a TEG. In some embodiments, the molecular species comprises, consists essentially of, or consists of a cholesterol linked to a TEG. In some embodiments, the molecular species is a cholesteryl ester. In some embodiments, the molecular species is a cholesteryl ester linked to a TEG. In some embodiments, the molecular species is, comprises, consists essentially of, or consists of N-cholesteryl-3-aminopropyl-triethyleneglycol-glyceryl-1-O-phosphodiester (3CholTEG).

In some embodiments, the molecular species is a lipid, a sterol, lipid moieties such as a cholesterol moiety, cholic acid, a thioether (e.g., hexyl-S-tritylthiol), a thiocholesterol, an aliphatic chain (e.g., dodecandiol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, an octadecylamine or hexylamino-carbonyl-oxycholesterol moiety, stearyl, C16 alkyl chain, bile acids, cholic acid, taurocholic acid, deoxycholate, oleyl litocholic acid, oleoyl cholenic acid, glycolipids, phospholipids, sphingolipids, isoprenoids, such as steroids, vitamins, such as vitamin E, saturated fatty acids, unsaturated fatty acids, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy576), Hoechst 33258 dye, psoralen, or ibuprofen.

In some embodiments, oligonucleotides (e.g., synthetic oligonucleotides) are anchored to the surface of a core through a molecular species. In some embodiments, the molecular species is tocopherols, sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

In some embodiments, a molecular species is connected to a synthetic oligonucleotide through a linker. In some embodiments, the linker is a spacer (e.g., a non-nucleotidic spacer). Non-limiting examples of a spacer are abasic residues (dSpacer), oligoethyleneglycol, such as triethyleneglycol (spacer 9 or iSp9; TEG) or hexaethylenegylcol (spacer 18 or iSp18; HEG), or alkane-diol (e.g., butanediol). In some embodiments, the synthetic oligonucleotide is attached to the spacer through a covalent bond (e.g., phosphodiester, phosphorodithioate or phosphorothioate bond). In some embodiments, the spacer does not comprise or consist of an oligonucleotide spacer. The spacer in some embodiments appears just once in the molecule or in some embodiments is incorporated several times (e.g., via phosphodiester, phosphorothioate, methylphosphonate, or amide linkages). In embodiments in which multiple spacer moieties are incorporated, the individual spacer moieties may be attached to one another and/or to the synthetic oligonucleotide via phosphodiester, phosphorothioate, methylphosphonate, or amide linkages. In some embodiments, the spacer comprises a TEG and/or a HEG. In some embodiments, the spacer comprises, consists essentially of, or consists of hexa(ethyleneglycol)phosphodiester-hexa(ethyleneglycol)phosphodiester (HEG-HEG). In some embodiments, the molecular species connected to the spacer comprises, consists essentially of, or consists of hexa(ethyleneglycol)phosphodiester-hexa(ethyleneglycol)phosphodiester-3-O—(N-cholesteryl-3-aminopropyl)-triethyleneglycol-glyceryl-1-O-phosphodiester (HEG-HEG-CholTEG or CholTEG-HEG-HEG). The HEG-HEG-CholTEG or CholTEG-HEG-HEG may be connected (e.g., directly or indirectly) to the 5′-end and/or the 3′-end of a nucleic acid or oligonucleotide (e.g., synthetic oligonucleotide) disclosed herein.

In some embodiments, kallikrein-related peptidase or “KLK” gene refers to an allele, alleles, a genomic locus or genomic loci corresponding to a KLK gene in an organism (e.g., KLKB1, KLK1, KLK2, KLK3, KLK4, KLK5, KLK6, KLK7, KLK8, KLK9, KLK10, KLK11, KLK12, KLK13, KLK14, or KLK15 in humans). In some embodiments, the KLK gene corresponds to the nucleic acid sequence of SEQ ID NO: 7477, SEQ ID NO: 7478, SEQ ID NO: 7575 or SEQ ID NO: 7576. In some embodiments, the KLK gene is KLK5 or KLK7.

In some embodiments, a KLK gene comprises, consists essentially of, or consists of a nucleic acid sequence of SEQ ID NO: 7477, SEQ ID NO: 7478, SEQ ID NO: 7575 or SEQ ID NO: 7576. In some embodiments, a KLK gene comprises a kallikrein-related peptidase genomic locus or a KLK polynucleotide. As disclosed herein, a “KLK gene product” refers to a KLK pre-mRNA, a KLK mRNA, a KLK coding sequence (e.g., a cDNA, synthetic RNA coding sequence, or synthetic DNA coding sequence), a KLK polypeptide, a KLK preprotein, or a KLK protein. In some embodiments, a KLK gene comprises a kallikrein-related peptidase 5 (KLK5) genomic locus or a KLK5 polynucleotide. In some embodiments, a “KLK gene product” refers to a KLK5 pre-mRNA, a KLK5 mRNA, a KLK5 coding sequence (e.g., a cDNA, synthetic RNA coding sequence, or synthetic DNA coding sequence), a KLK5 polypeptide, a KLK5 preprotein, or a KLK5 protein. In some embodiments, a KLK gene comprises a kallikrein-related peptidase 7 (KLK7) genomic locus or a KLK7 polynucleotide. In some embodiments, a “KLK gene product” refers to a KLK7 pre-mRNA, a KLK7 mRNA, a KLK7 coding sequence (e.g., a cDNA, synthetic RNA coding sequence, or synthetic DNA coding sequence), a KLK7 polypeptide, a KLK7 preprotein, or a KLK7 protein.

In some embodiments, a region of a kallikrein-related peptidase (KLK) gene refers to a segment of an allele, a genomic locus, or a polynucleotide corresponding to a KLK gene (e.g., an allele, genomic locus, or polynucleotide corresponding to human KLKB1, KLK1, KLK2, KLK3, KLK4, KLK5, KLK6, KLK7, KLK8, KLK9, KLK10, KLK11, KLK12, KLK13, KLK14, or KLK15). In some embodiments, the region is a nucleotide sequence within at least one of a 5′-UTR, a 3′-UTR, an enhancer region, a silencer region, a regulatory region, an intron, an exon, a promoter region, a coding region, a termination region, or any combination thereof. As disclosed herein, a “region of a KLK gene product” or equivalent phrases refer to a region in a KLK pre-mRNA, a KLK mRNA, a KLK coding sequence (e.g., a cDNA, synthetic RNA coding sequence, or synthetic DNA coding sequence), a KLK polypeptide, a KLK preprotein, or a KLK protein. In some embodiments, a “region of a KLK gene product” is a region of a KLK5 gene product, such as a region in a KLK5 pre-mRNA, a KLK5 mRNA, a KLK5 coding sequence (e.g., a cDNA, synthetic RNA coding sequence, or synthetic DNA coding sequence), a KLK5 polypeptide, a KLK5 preprotein, or a KLK5 protein. In some embodiments, a “region of a KLK gene product” is a region of a KLK7 gene product, such as a region in a KLK7 pre-mRNA, a KLK7 mRNA, a KLK7 coding sequence (e.g., a cDNA, synthetic RNA coding sequence, or synthetic DNA coding sequence), a KLK7 polypeptide, a KLK7 preprotein, or a KLK7 protein.

In some embodiments, the region of a KLK gene or a KLK gene product is within an exon of a KLK5 or KLK7 gene or gene product. In some embodiments, the region is within exon 1 (nucleotides 1-292 of SEQ ID NO: 7575), exon 2 (nucleotides 293-376 of SEQ ID NO: 7575), exon 3 (nucleotides 377-638 of SEQ ID NO: 7575), exon 4 (nucleotides 639-895 of SEQ ID NO; 7575), exon 5 (nucleotides 896-1029 of SEQ ID NO: 7575), or exon 6 (nucleotides 1030-1513 of SEQ ID NO; 7575) of a KLK5 gene or gene product. In some embodiments, the region is within exon 1 (nucleotides 1-67 of SEQ ID NO: 7576), exon 2 (nucleotides 68-198 of SEQ ID NO: 7576), exon 3 (nucleotides 199-346 of SEQ ID NO: 7576), exon 4 (nucleotides 347-594 of SEQ ID NO: 7576), exon 5 (nucleotides 595-731 of SEQ ID NO: 7576), or exon 6 (nucleotides 732-1955 of SEQ ID NO: 7576) of a KLK7 gene or gene product.

In some embodiments, the region is within exon 6 of a KLK5 gene or gene product. In some embodiments, the region is within exon 5 of a KLK7 gene or gene product. In some embodiments, the region is within exon 1, exon 3, exon 4, or exon 6 of a KLK7 gene or gene product. In some embodiments, the region of a KLK gene or a KLK gene product is partially or spans within two exons of a KLK5 or KLK7 gene or gene product. As a non-limiting example, the region may be partially within exon 5 and partially within exon 6 of a KLK7 gene product. In some embodiments, the region is within a 5′UTR or a 3′-UTR of an exon or a coding sequence of a KLK gene or KLK gene product. As a non-limiting example, the region may be within the 3′-UTR of exon 5 or exon 6 of a KLK5 gene product, or the region may be within the 3′-UTR of exon 6 of a KLK7 gene product. In some embodiments, the region may be within the 5′-UTR of exon 1 or exon 2 of a KLK5 gene product, or the region may be within the 5′-UTR of exon 1 or exon 2 of a KLK7 gene product.

In some embodiments, the region of KLK gene or a KLK gene product is within a region of SEQ ID NO: 7477, SEQ ID NO: 7478, SEQ ID NO: 7575 or SEQ ID NO: 7576. In some embodiments, the region is entirely within, partially within, or approximately within the range of nucleotides 103-142 (e.g., nucleotides 110-136), nucleotides 305-344, nucleotides 820-920 (e.g., nucleotides 875-902), nucleotides 1152-1191 (e.g., nucleotides 1158-1185), or nucleotides 1259-1426 (e.g., nucleotides 1265-1300 or nucleotides 1393-1418) of SEQ ID NO: 7575. In some embodiments, the region is within a corresponding region of SEQ ID NO: 7477 or SEQ ID NO: 7577. In some embodiments, the region is within the range of nucleotides 1259-1426 of SEQ ID NO: 7575. In some embodiments, the region is entirely within, partially within, or approximately within the range of nucleotides 41-69, nucleotides 262-375 (e.g., nucleotides 265-290, nucleotides 266-355, nucleotides 302-327, or nucleotides 347-372), nucleotides 590-621 (e.g., nucleotides 593-618), nucleotides 722-753 (e.g., nucleotides 725-750), nucleotides 1460-1491 (e.g., nucleotides 1463-1488), or nucleotides 1616-1647 (e.g., nucleotides 1619-1644) of SEQ ID NO: 7576. In some embodiments, the region is within a corresponding region of SEQ ID NO: 7478 or SEQ ID NO: 7578. In some embodiments, the region is within the range of nucleotides 590-621 of SEQ ID NO: 7576.

As disclosed herein, a “spherical nucleic acid (SNA)” refers to a three-dimensional arrangement of nucleic acids or oligonucleotides, such as synthetic oligonucleotides, comprising an oligonucleotide shell, with radially arranged oligonucleotides on the exterior of a core. In some embodiments, the core is a hollow core produced by a three-dimensional arrangement of molecules which form the outer boundary of the core. For instance, the molecules may be in the form of a lipid layer (e.g., lipid monolayer or lipid bilayer), which has a hollow center. In some embodiments, the molecules may be in the form of lipids, such as amphipathic lipids (e.g., sterols), which are linked to or associated with, either directly or indirectly, an end of the oligonucleotide (e.g., synthetic oligonucleotide). In some embodiments, tocopherols or sterols, such as cholesterol, linked (e.g., indirectly attached) to an end of an oligonucleotide (e.g., a synthetic oligonucleotide) may associate with the outer surface of a core (e.g., a hollow core), such that the oligonucleotides radiate outward from the core.

In some embodiments, an SNA comprises an oligonucleotide shell comprising a synthetic oligonucleotide and a core. In some embodiments, a synthetic oligonucleotide is associated with the core (e.g., liposome core) through a covalent or non-covalent interaction. In some embodiments, a synthetic oligonucleotide is associated with the exterior surface of the core. In some embodiments, the synthetic oligonucleotides are associated with the core (e.g., exterior surface) through a molecular species via, for instance, a hydrophobic interaction. In some embodiments, the synthetic oligonucleotide is associated with a molecular species (e.g., a hydrophobic group), either directly or indirectly. In some embodiments, the synthetic oligonucleotide is indirectly associated with or indirectly attached to a molecular species (e.g., hydrophobic group) through a spacer. In some embodiments, the molecular species (e.g., hydrophobic group) is associated with the core. In some embodiments, the core is a liposome core. In some embodiments, the liposome core comprises a lipid bilayer. In some embodiments, the synthetic oligonucleotide is covalently attached to one or more lipids of the lipid layer. In some embodiments, the synthetic oligonucleotide is not covalently attached to one or more lipids of the lipid layer. In some embodiments, the synthetic oligonucleotide is covalently attached to a molecular species.

In some embodiments, an SNA disclosed herein comprises a first synthetic oligonucleotide comprising a first nucleic acid sequence and a second synthetic oligonucleotide comprising a second nucleic acid sequence. In some embodiments, a nucleic acid sequence (e.g., a nucleic acid sequence of a synthetic oligonucleotide) is indirectly attached to a molecular species. In some embodiments, a first molecular species is indirectly attached to a first nucleic acid sequence (e.g., a first nucleic acid sequence of a first synthetic oligonucleotide) and a second molecular species is indirectly attached to a second nucleic acid sequence (e.g., a second nucleic acid sequence of a second synthetic oligonucleotide). In some embodiments, the first molecular species is indirectly attached to the 3′-end of the first nucleic acid sequence and the second molecular species is indirectly attached to the 5′-end of the second nucleic acid sequence.

In some embodiments, the first molecular species is indirectly attached to the 5′-end of the first nucleic acid sequence and the second molecular species is indirectly attached to the 3′-end of the second nucleic acid sequence. In some embodiments, the first molecular species is indirectly attached to the 5′-end of the first synthetic oligonucleotide and the second molecular species is indirectly attached to the 5′-end of the second synthetic oligonucleotide. In some embodiments, the first molecular species is indirectly attached to the 3′-end of the first synthetic oligonucleotide and the second molecular species is indirectly attached to the 3′-end of the second synthetic oligonucleotide.

In some embodiments, an SNA disclosed herein comprises a first synthetic oligonucleotide comprising a first nucleic acid sequence and a second synthetic oligonucleotide comprising a second nucleic acid sequence. In some embodiments, a nucleic acid sequence (e.g., a nucleic acid sequence of a synthetic oligonucleotide) is directly attached to a molecular species. In some embodiments, a first molecular species is directly attached to a first nucleic acid sequence (e.g., a first nucleic acid sequence of a first synthetic oligonucleotide) and a second molecular species is directly attached to a second nucleic acid sequence (e.g., a second nucleic acid sequence of a second synthetic oligonucleotide). In some embodiments, the first molecular species is directly attached to the 3′-end of the first nucleic acid sequence and the second molecular species is directly attached to the 5′-end of the second nucleic acid sequence. In some embodiments, the first molecular species is directly attached to the 5′-end of the first nucleic acid sequence and the second molecular species is directly attached to the 3′-end of the second nucleic acid sequence. In some embodiments, the first molecular species is directly attached to the 5′-end of the first synthetic oligonucleotide and the second molecular species is directly attached to the 5′-end of the second synthetic oligonucleotide. In some embodiments, the first molecular species is directly attached to the 3′-end of the first synthetic oligonucleotide and the second molecular species is directly attached to the 3′-end of the second synthetic oligonucleotide.

In some embodiments, the core is a solid core or a hollow core.

In some embodiments, a solid core is a spherical-shaped material with or without a hollow center. As disclosed herein, a “spherical shape” refers to a general shape and does not imply or is not limited to a perfect sphere or round shape. In some embodiments, a spherical shape includes imperfections. In some embodiments, the core comprises, consists essentially of, or consists of a metal core (e.g., any metal). Non-limiting examples of metals include gold, silver, platinum, aluminum, palladium, copper, cobalt, indium, nickel and mixtures thereof. In some embodiments, the core comprises gold or is a gold core. In some embodiments, the core can be a lattice structure including degradable gold. In some embodiments, the core may comprise semiconductor and/or magnetic materials. Solid cores can be constructed from a wide variety of materials known to those skilled in the art including but not limited to: noble metals (gold, silver), transition metals (iron, cobalt) and metal oxides (silica). A solid core may be inert, paramagnetic, or superparamagnetic. The solid cores can be constructed from either pure compositions of described materials, or in combinations of mixtures of any number of materials, or in layered compositions of materials. In some embodiments, a solid core can be composed of a polymeric core such as amphiphilic block copolymers, hydrophobic polymers such as polystyrene, poly(lactic acid), poly(lactic co-glycolic acid), poly(glycolic acid), poly(caprolactone) and other biocompatible polymers known to those skilled in the art. In some embodiments, the core comprises, consists essentially of, or consists of a solid core or a semi-solid core.

In some embodiments, a hollow core has at least some space in the center region of a shell material. In some embodiments, a hollow core is a liposome core. A liposome core as used herein refers to a centrally located core compartment formed by a component of the lipids or phospholipids that form a lipid bilayer. “Liposomes” are artificial, self-closed vesicular structures of various sizes and shapes, where one or several membranes encapsulate an aqueous core. Most typically liposome membranes are formed from lipid bilayer membranes, where the hydrophilic head groups are oriented towards aqueous environments, and the lipid chains are oriented away from aqueous environments. Liposomes can be formed as well from other amphiphilic monomeric and polymeric molecules, such as polymers, like block copolymers, or polypeptides. Unilamellar vesicles are liposomes defined by a single membrane enclosing an aqueous space. In contrast, oligo- or multilamellar vesicles are built up of several membranes. Typically, the membranes are roughly 4 nm thick and are composed of amphiphilic lipids, such as phospholipids, of natural or synthetic origin. Optionally, the membrane properties can be modified by the incorporation of other lipids such as sterols or cholic acid derivatives.

The lipid bilayer is composed of two layers of lipids or lipid molecules. Each lipid or lipid molecule in a layer is oriented substantially parallel to adjacent lipids or lipid molecules, and two layers of lipids or lipid molecules that form a bilayer have the polar ends of their molecules exposed to the aqueous phase and the non-polar ends adjacent to each other. The two lipid layers that form the lipid bilayer are substantially parallel to one another. The central aqueous region of the liposomal core may be empty or filled fully or partially with water, an aqueous emulsion, oligonucleotides, or other therapeutic or diagnostic agents or aqueous solutions thereof.

A lipid bilayer or liposome core can be constructed from one or more lipids known to those in the art including but not limited to: 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE), sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives. In some embodiments, the liposome core comprises, consists essentially of, or consists of 1,2-dioleoyl-sn-glycero-3-phosphocholine (DOPC).

As disclosed herein, a “lipid” refers to its conventional sense as a generic term encompassing fats, lipids, and alcohol-ether-soluble constituents of protoplasm, which are insoluble in water. Lipids usually consist of a hydrophilic and a hydrophobic moiety. In water, lipids can self-organize to form bilayer membranes, where the hydrophilic moieties (head groups) are oriented towards the aqueous phase, and the lipophilic moieties (acyl chains) are embedded in the hydrophobic region between the two hydrophilic layers comprised within the bilayer. Lipids can also comprise two hydrophilic moieties (bola amphiphiles). In that case, membranes may be formed from a single lipid layer, and not a bilayer. Typical non-limiting examples of lipids are fats, fatty oils, essential oils, waxes, steroids, sterols, phospholipids, glycolipids, sulpholipids, aminolipids, chromolipids, and fatty acids. The term encompasses both naturally occurring and synthetic lipids.

In some embodiments, the lipids are steroids, sterols (e.g., cholesterol), phospholipids, including phosphatidyl, phosphatidylcholines and phosphatidylethanolamines and sphingomyelins. Where there are fatty acids, they could be about 12-24 carbon chains in length, containing up to 6 double bonds. The fatty acids are linked to a backbone, which may be derived from glycerol. The fatty acids within one lipid can be different (asymmetric), or there may be only 1 type of fatty acid chain present (e.g., lysolecithins). Mixed formulations are also possible, particularly when the non-cationic lipids are derived from natural sources, such as lecithins (phosphatidylcholines) purified from egg yolk, bovine heart, brain, liver or soybean.

In some embodiments, the SNA includes a neutral lipid. The neutral lipid may be, for example, 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE), any related phosphatidylcholine or neutral lipids available from commercial vendors. In some embodiments, the SNA includes 1,2-dioleoyl-sn-glycero-3-phosphocholine (DOPC).

In some embodiments, the core is a niosome core. A noisome is a vesicle formed from non-ionic surfactant oriented in a bilayer. Niosomes commonly have cholesterol added as an excipient, but other lipid-based and non-lipid-based constituents can also be included. Methods for preparation of niosomes are known in the art. In some embodiments polyethylene glycol (PEG) is included during or following niosome preparation. Niosome vesicles are structurally and functionally analogous to liposomes, but are based on non-ionic surfactant rather than lipid as the primary constituent. Common non-ionic surfactants used include sorbitans (spans) or polysorbates (tween); however, a wide variety of non-ionic surfactants can be used to prepare niosomes.

As used herein, “conjugated,” “attached,” “linked,” or “directly attached” means two entities stably bound to one another by any physiochemical means. It is important that the nature of the attachment is such that it does not impair substantially the effectiveness of either entity. Keeping these parameters in mind, any covalent or non-covalent linkage known to those of ordinary skill in the art may be employed. In some embodiments, covalent linkage is preferred. Noncovalent conjugation includes hydrophobic interactions, ionic interactions, high affinity interactions such as biotin avidin and biotin streptavidin complexation and other affinity interactions. Such means and methods of attachment are well known to those of ordinary skill in the art.

In some embodiments, an SNA comprises an oligonucleotide shell. In some embodiments, an oligonucleotide shell includes one or more oligonucleotides (e.g., synthetic oligonucleotides) on the exterior of the core. In some embodiments, the oligonucleotide shell comprises, consists essentially of, or consists of one, two, three, four, five, six, seven, eight, nine, 10, 11, 12, 13, 14, 15, 16, 17, 18, 19, 20, about 25, about 30, about 40, about 50, about 60, about 70, about 80, about 90, about 100, about 150, about 200, about 250, about 300, about 400, about 500, about 600, about 700, about 800, about 900, about 1000, about 1500, about 2000, about 3000, about 4000, about 5000, about 10,000, about 15,000, about 20,000 or more synthetic oligonucleotides, or any range or combination thereof. In some embodiments, the oligonucleotide shell comprises, consists essentially of, or consists of 2-1000, 2-900, 2-800, 2-700, 2-600, 2-500, 2-400, 2-300, 2-200, 2-100, 2-90, 2-80, 2-70, 2-60, 2-50, 2-45, 2-40, 2-35, 2-30, 2-25, 2-20, 2-15 or 2-10 oligonucleotides (e.g., synthetic oligonucleotides). In some embodiments, the oligonucleotide shell comprises, consists essentially of, or consists of 5-100, 10-100, 15-100, 20-100, 25-100, 50-100, 5-50, 10-50, 15-50, 20-50, 25-50, 5-40, 10-40, 15-40, 20-40, 25-40, 5-30, 10-30, 15-30, 20-30, 25-30, 5-25, 10-25, 15-25 or 20-25 oligonucleotides (e.g., synthetic oligonucleotides). In some embodiments, the oligonucleotide shell comprises, consists essentially of, or consists of 30 or about 30 oligonucleotides (e.g., synthetic oligonucleotides).

In some embodiments, an SNA disclosed herein comprises, consists essentially of, or consists of lipids or lipid molecules and oligonucleotides (e.g., synthetic oligonucleotides) at a molar ratio between 100 to 1 and 10 to 1, between 100 to 1 and 20 to 1, between 100 to 1 and 30 to 1, between 100 to 1 and 40 to 1, between 100 to 1 and 50 to 1, between 100 to 1 and 60 to 1, between 100 to 1 and 70 to 1, between 100 to 1 and 80 to 1, between 100 to 1 and 90 to 1, between 90 to 1 and 10 to 1, between 90 to 1 and 20 to 1, between 90 to 1 and 30 to 1, between 90 to 1 and 40 to 1, between 90 to 1 and 50 to 1, between 90 to 1 and 60 to 1, between 90 to 1 and 70 to 1, between 90 to 1 and 80 to 1, between 80 to 1 and 10 to 1, between 80 to 1 and 20 to 1, between 80 to 1 and 30 to 1, between 80 to 1 and 40 to 1, between 80 to 1 and 50 to 1, between 80 to 1 and 60 to 1, between 80 to 1 and 70 to 1, between 70 to 1 and 10 to 1, between 70 to 1 and 20 to 1, between 70 to 1 and 30 to 1, between 70 to 1 and 40 to 1, between 70 to 1 and 50 to 1, between 70 to 1 and 60 to 1, between 60 to 1 and 10 to 1, between 60 to 1 and 20 to 1, between 60 to 1 and 30 to 1, between 60 to 1 and 40 to 1, between 60 to 1 and 50 to 1, between 50 to 1 and 10 to 1, between 50 to 1 and 20 to 1, between 50 to 1 and 30 to 1, between 50 to 1 and 40 to 1, between 40 to 1 and 10 to 1, between 40 to 1 and 20 to 1, between 40 to 1 and 30 to 1, between 30 to 1 and 10 to 1, between 30 to 1 and 20 to 1 of lipids or lipid molecules to oligonucleotide (e.g., synthetic oligonucleotide), or any range or combination thereof.

In some embodiments, an SNA disclosed herein comprises lipids or lipid molecules and oligonucleotides (e.g., synthetic oligonucleotides) at a molar ratio of at least or about 10 to 1, 20 to 1, 30 to 1, 40 to 1, 50 to 1, 60 to 1, 70 to 1, 80 to 1, 90 to 1, or 100 to 1 of lipids to oligonucleotide (e.g., synthetic oligonucleotide), or any range or combination thereof. In some embodiments, an SNA disclosed herein comprises lipids or lipid molecules and oligonucleotides (e.g., synthetic oligonucleotides) at a molar ratio of between 55 to 1 and 45 to 1 of lipid or lipid molecules to oligonucleotide. In some embodiments, an SNA disclosed herein comprises lipids or lipid molecules and oligonucleotides (e.g., synthetic oligonucleotides) at a molar ratio of or about 50 to 1 of lipid to oligonucleotide (e.g., synthetic oligonucleotide). In some embodiments, the oligonucleotide portion of the lipids or lipid molecules to oligonucleotide ratio is divided between more than one population of oligonucleotides (e.g., a first population of synthetic oligonucleotides, a second population of synthetic oligonucleotides, etc.).

In some embodiments, an SNA comprising a first population of synthetic oligonucleotides and a second population of synthetic oligonucleotides may comprise lipids or lipid molecules at a molar ratio of 50 to 0.5 to 0.5 of lipids or lipid molecules to first population of synthetic oligonucleotides to second population of synthetic oligonucleotides. In some embodiments, an SNA comprising a first population of synthetic oligonucleotides and a second population of synthetic oligonucleotides may comprise different amounts of the first population of synthetic oligonucleotides and the second population of synthetic oligonucleotides, such that the molar ratio of lipids or lipid molecules to the first population of synthetic oligonucleotides to the second population of synthetic oligonucleotides is, for example, 50 to 0.1 to 0.9, 50 to 0.2 to 0.8, 50 to 0.3 to 0.7, 50 to 0.4 to 0.6, 50 to 0.6 to 0.4, 50 to 0.7 to 0.3, 50 to 0.8 to 0.2, or 50 to 0.9 to 0.1, or any range or combination thereof, of lipid molecules to first population of synthetic oligonucleotides to second population of synthetic oligonucleotides.

In some embodiments, the first population of synthetic oligonucleotides comprises one or more first synthetic oligonucleotides disclosed herein complementary to a region of a first KLK gene that is a KLK5 gene and/or first gene product that is a KLK5 gene product. In some embodiments, the second population of synthetic oligonucleotides comprises one or more second synthetic oligonucleotides disclosed herein complementary to or sufficiently complementary to a region of a second KLK gene that is a KLK7 gene and/or second gene product that is a KLK7 gene product.

In some embodiments, the first population of synthetic oligonucleotides comprises one or more first synthetic oligonucleotides disclosed herein comprising a nucleic acid sequence complementary to or sufficiently complementary to a region of a first KLK gene that is a KLK7 gene and/or first gene product that is a KLK7 gene product. In some embodiments, the second population of synthetic oligonucleotides comprises one or more second synthetic oligonucleotides disclosed herein comprising a nucleic acid sequence complementary to or sufficiently complementary to a region of a second KLK gene that is a KLK5 gene and/or second gene product that is a KLK5 gene product.

In some embodiments, the oligonucleotides (e.g., synthetic oligonucleotides) are on the exterior surface of the core (e.g., liposome core). In some embodiments, at least one oligonucleotide (e.g., a synthetic oligonucleotide) has its 5′-terminus exposed on the exterior surface away from the core (e.g., the 5′-terminus is located at the end of the oligonucleotide distal from the core). In some embodiments, all of the oligonucleotides (e.g., synthetic oligonucleotides) in an SNA have their 5′-termini exposed on the exterior surface away from the core (e.g., the 5′-termini are located at the ends of the oligonucleotides distal from the core). In some embodiments, at least one oligonucleotide (e.g., a synthetic oligonucleotide) has its 3′-terminus exposed on the exterior surface away from the core (e.g., the 3′-terminus is located at the end of the oligonucleotide distal from the core). In some embodiments, all of the oligonucleotides (e.g., synthetic oligonucleotides) in an SNA have their 3′-termini exposed on the exterior surface away from the core (e.g., the 3′-termini are located at the ends of the oligonucleotides distal from the core). In some embodiments, the SNA does not include an oligonucleotide (e.g., a synthetic oligonucleotide) inside the core (e.g., liposome core).

In some embodiments, an SNA comprises a first synthetic oligonucleotide and a second synthetic oligonucleotide. In some embodiments, the 5′-terminus of the first synthetic oligonucleotide is exposed on the exterior surface of the SNA away from the core, and the 3′-terminus of the second synthetic oligonucleotide is exposed on the surface of the SNA away from the core. In some embodiments, the 3′-terminus of the first synthetic oligonucleotide is exposed on the exterior surface of the SNA away from the core, and the 5′-terminus of the second synthetic oligonucleotide is exposed on the surface of the SNA away from the core. In some embodiments, the 3′-terminus of the first synthetic oligonucleotide is exposed on the exterior surface of the SNA away from the core, and the 3′-terminus of the second synthetic oligonucleotide is exposed on the surface of the SNA away from the core. In some embodiments, the 5′-terminus of the first synthetic oligonucleotide is exposed on the exterior surface of the SNA away from the core, and the 5′-terminus of the second synthetic oligonucleotide is exposed on the surface of the SNA away from the core.

In some embodiments, two or more of the oligonucleotides (e.g., synthetic oligonucleotides) in an SNA are crosslinked to one another. In some embodiments, all of the oligonucleotides (e.g., synthetic oligonucleotides) in an SNA are crosslinked to one or more additional oligonucleotides. In some embodiments, the oligonucleotides (e.g., synthetic oligonucleotides) in an SNA are not crosslinked.

In some embodiments, an SNA disclosed herein has a diameter of, or a population or plurality of SNAs disclosed herein has a mean diameter of about 10 to about 150 nm. In some embodiments, the mean diameter of the population of SNAs is from or about 15 nm to about 100 nm, about 20 nm to about 100 nm, about 25 nm to about 100 nm, about 15 nm to about 50 nm, about 20 nm to about 50 nm, about 10 nm to about 70 nm, about 15 nm to about 70 nm about 20 nm to about 70 nm, about 10 nm to about 30 nm, about 15 nm to about 30 nm, about 20 nm to about 30 nm, about 10 nm to about 40 nm, about 15 nm to about 40 nm, about 20 nm to about 40 nm, about 10 nm to about 80 nm, about 15 nm to about 80 nm, or about 20 nm to about 80 nm.

In some embodiments, the mean diameter of the population of SNAs is from about 15 nm to about 45 nm. In some embodiments, an SNA disclosed herein has a diameter, or a population of SNAs disclosed herein has a mean diameter of or about 10 nm, about 15 nm, about 20 nm, about 30 nm, about 40 nm, about 50 nm, about 60 nm, about 70 nm, about 80 nm, about 90 nm, or about 100 nm. In some embodiments, an SNA disclosed herein has a diameter, or a population or a plurality of SNAs disclosed herein have a mean diameter of 30 nm or about 30 nm.

In some embodiments, the core (e.g., liposome core) of an SNA disclosed herein has a diameter of, or the cores (e.g., liposome cores) of a population of SNAs disclosed herein has a mean diameter of about 10 to about 150 nm. In some embodiments, the diameter of the core or the mean diameter of the population of cores is from about 15 nm to about 100 nm, about 20 nm to about 100 nm, about 25 nm to about 100 nm, about 15 nm to about 50 nm, about 20 nm to about 50 nm, about 10 nm to about 70 nm, about 15 nm to about 70 nm, about 20 nm to about 70 nm, about 10 nm to about 30 nm, about 15 nm to about 30 nm, about 20 nm to about 30 nm, about 10 nm to about 40 nm, about 15 nm to about 40 nm, about 20 nm to about 40 nm, about 10 nm to about 80 nm, about 15 nm to about 80 nm, or about 20 nm to about 80 nm. In some embodiments, the core (e.g., liposome core) of an SNA disclosed herein has a diameter of, or the cores (e.g., liposome cores) of a population of SNAs disclosed herein has a mean diameter of about 15 nm to about 45 nm. In some embodiments, the core (e.g., liposome core) of an SNA disclosed herein has a diameter of, or the cores (e.g., liposome cores) of a population of SNAs disclosed herein has a mean diameter of about 10 nm, about 15 nm, about 20 nm, about 30 nm, about 40 nm, about 50 nm, about 60 nm, about 70 nm, about 80 nm, about 90 nm, or about 100 nm. In some embodiments, the core (e.g., liposome core) of an SNA disclosed herein has a diameter of, or the cores (e.g., liposome cores) of a population of SNAs disclosed herein has a mean diameter of about 30 nm.

In some embodiments, the core (e.g., liposome core) of an SNA disclosed herein has a diameter, or the cores (e.g., liposome cores) of a population of SNAs disclosed herein has a mean diameter of about or less than about 10 nm, 15 nm, 20 nm, 25 nm, 30 nm, 35 nm, and/or 40 nm, or any range or combination thereof. In some embodiments, the core (e.g., liposome core) of an SNA disclosed herein has a diameter, or the cores (e.g., liposome cores) of a population of SNAs disclosed herein has a mean diameter of about or less than 40 nm.

The SNAs disclosed herein may be stable self-assembling nanostructures. For instance, the SNA may comprise an oligonucleotide (e.g., a synthetic oligonucleotide) of 18-21 nucleosides in length having a sequence disclosed herein, wherein a hydrophobic group at the 3′ or 5′ terminus of the oligonucleotide self-associates to form the core or associates with the core of the nanostructure in water or other suitable solvents. A hydrophobic group as used herein may include cholesterol, a cholesteryl or modified cholesteryl residue, adamantine, dihydrotesterone, long chain alkyl, long chain alkenyl, long chain alkynyl, olely-lithocholic, cholenic, oleoyl-cholenic, palmityl, heptadecyl, myrisityl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen.

As disclosed herein, a disease or disorder refers to a disease or disorder in a subject. In some embodiments, the disease or disorder is an ichthyosis disease or disorder. In some embodiments, the disease or disorder is associated with or caused by dysregulation of protease activity. In some embodiments, the disease or disorder is associated with or caused by dysregulation of the activity of a kallikrein-related peptidase or kallikrein-related peptidases. In some embodiments, the disease or disorder is associated with or caused by dysregulation of KLK5 protein or activity, KLK7 protein or activity, or KLK5 protein or activity and KLK7 protein or activity. In some embodiments, the disease or disorder is associated with or caused decreased or absent function of a protease inhibitor or protease inhibitors. In some embodiments, the disease or disorder is associated with or caused by decreased or absent function of lympho-epithelial Kazal-type-related inhibitor (LEKTI, sometimes referred to as LEKT1). In some embodiments, the disease or disorder is associated with or caused by a mutation or mutations in a gene encoding LEKTI. In some embodiments, the disease or disorder is associated with or caused by a mutation or mutations in the serine protease inhibitor Kazal-type 5 (SPINK5) gene.

In some embodiments, the disease or disorder is NS.

In some embodiments, a “cell” refers to a cell obtained from a subject or existing within a subject. In some embodiments, the cell is a cell which produces peptidases, such as kallikrein-related peptidases. The cell may be a skin cell (e.g., an epithelial cell or a dermal cell, including but not limited to a keratinocyte, such as a granular keratinocyte, a basal cell, a sebaceous gland cell, a sweat gland cell, a dermal root sheath cell, an inner root sheet cell, or an outer root sheath cell), an immune cell (e.g., an innate immune cell or an adaptive immune cell, including but not limited to a neutrophil, a macrophage, a T cell, a B cell, a dendritic cell, or a natural killer cell).

In some embodiments, the cell is from a subject having a disease or disorder, such as a skin disease or disorder disclosed herein. In some embodiments, the cell is from a subject having an ichthyosis disease or disorder. In some embodiments, the cell is from a subject having NS. In some embodiments, the cell is a keratinocyte.

In some embodiments, the cell is contacted with an oligonucleotide (e.g., synthetic oligonucleotide), a SNA, or a composition thereof (e.g., pharmaceutical composition thereof) disclosed herein at a concentration of at least 0.001 nM, at least 0.01 nM, at least 0.1 nM, at least 1 nM, at least 10 nM, at least 100 nM, at least 1000 nM, at least 10 μM, at least 100 μM, at least 1000 μM, or above 1000 μM. In some embodiments, the cell is contacted with oligonucleotide (e.g., synthetic oligonucleotide), a SNA, or a composition (e.g., pharmaceutical composition) disclosed herein at a concentration range of 0.001 nM to 0.01 nM, 0.01 nM to 0.1 nM, 0.1 nM to 1 nM, 1 nM to 10 nM, 10 nM to 100 nM, 100 nM to 1000 nM, 1000 nM to 10 μM, 10 μM to 100 μM, or 100 μM to 1000 μM. In some embodiments, the cell is contacted with oligonucleotide (e.g., synthetic oligonucleotide), a SNA, or a composition (e.g., pharmaceutical composition) disclosed herein at a concentration of 0.001 nM, 0.01 nM, 0.1 nM, 1 nM, 10 nM, 100 nM, 1000 nM, 10 μM, 100 μM, 1000 μM or above 1000 μM. In some embodiments, the concentration refers to the total weight or total mass of an oligonucleotide (e.g., synthetic oligonucleotide) disclosed herein in a volume of solution. In a non-limiting example, a concentration of 0.001 nM refers to 0.001 nmoles of oligonucleotide (e.g., synthetic oligonucleotide) per liter of solution (e.g., pharmaceutically acceptable carrier). In some embodiments, the concentration refers to the total weight or total mass of an SNA disclosed herein in a volume of solution. In a non-limiting example, a concentration of 0.001 nM refers to 0.001 nM of SNA per liter of solution (e.g., pharmaceutically acceptable carrier).

As disclosed herein, to “ameliorate” or “eliminate” a disease or a symptom of a disease refers to decreasing the severity of the disease or the symptom or removing evidence of the disease or the symptom in a subject. In some instances, ameliorating or eliminating a disease or symptom may refer to a change in a metric associated with the disease or symptom as evaluated by a medical professional or as measured by a laboratory test. In some instances, ameliorating or eliminating a disease, disorder or symptom may refer to a change in a metric associated with the disease, disorder or symptom as reported by the subject having the disease or symptom. In some instances, ameliorating or eliminating a disease or symptom may refer to a decrease or elimination of pain associated with the disease, disorder or symptom.

In some embodiments, an oligonucleotide (e.g., synthetic oligonucleotide), SNA or pharmaceutically acceptable salt thereof disclosed herein is delivered to the epidermis. In some embodiments, an oligonucleotide (e.g., synthetic oligonucleotide), SNA or pharmaceutically acceptable salt thereof is delivered to at least one skin component chosen from the upper stratum granulosum, stratum corneum, hair follicle epithelia and sweat glands.

As disclosed herein, a “second therapeutic agent” refers to a therapeutic agent other than an oligonucleotide (e.g., synthetic oligonucleotide), SNA or composition disclosed herein. The second therapeutic agent may refer to any composition, compound, or device that is administered to or utilized on a subject having a disease or disorder, which may enhance the therapeutic effect of the synthetic oligonucleotide or SNA disclosed herein.

In some embodiments, the second therapeutic agent is a topical steroid or two or more topical steroids. In some embodiments, the second therapeutic agent is a topical corticosteroid. In some embodiments, the corticosteroid (e.g., topical corticosteroid) is clobetasol proprionate, betametasone diproprionate, fluocinolone acetonide or hydrocortisone. In some embodiments, the second therapeutic agent is a combination of two or more therapeutic agents. In some embodiments, the second therapeutic agent is a corticosteroid, such as a glucocorticoid-related steroid. In some embodiments, the second therapeutic agent is an anti-fungal agent. In some embodiments, the second therapeutic agent (e.g., steroid or corticosteroid) is administered topically, orally or by injection.

In some embodiments, the second therapeutic agent is a biological response modifier or biologic, which targets and inhibits an inflammatory cytokine (e.g., tumor necrosis factor-alpha or TNF-alpha) that plays a key role in immune cell recruitment and activation. In some embodiments, the second therapeutic agent is a topical immunomodulator (e.g., tacrolimus, pimecrolimus, etc.).

In some embodiments, the second therapeutic agent is an antioxidant. In some embodiments, the antioxidant is a synthetic antioxidant. In some embodiments, the antioxidant is a natural or botanically-based antioxidant. In some embodiments, the natural or botanically-based antioxidant is curcumin (e.g., found in turmeric and curry), quercetin (e.g., found in apples), epigallocatechin gallate (e.g., found in green tea), gallic acid, or bisabolol (e.g., found in chamomile). In some embodiments, the antioxidant is resveratrol. In some embodiments, the second therapeutic agent is a vitamin C derivative or a vitamin A derivative. In some embodiments, the natural or botanically-based antioxidant is a small phenolic anti-inflammatory compound found in basil, nutmeg or cheese. In some embodiments, the second therapeutic agent (e.g., natural or botanically-based antioxidant) has a beneficial therapeutic effect in a keratinocyte.

In some embodiments, the subject is a mammal. In some embodiments, the subject is a primate. In some embodiments, the subject is a human. In some embodiments, the mammal is a vertebrate animal including, but not limited to, a mouse, rat, dog, cat, horse, cow, pig, sheep, goat, turkey, chicken, monkey, fish (e.g., aquaculture species, salmon, etc.). Thus, the disclosure herein can be used to treat diseases or disorders, such as a skin disease, (e.g., chronic skin disease, such as a chronic inflammatory skin disease, an acute skin disease, eczema, rosacea, psoriasis, seborrheic dermatitis, allergic contact dermatitis, an ichthyosis disease, such as NS, etc.) in human or non-human subjects.

The disclosure herein can be used to treat a skin disease in a human or non-human subject. The disclosure herein can be used to treat a chronic skin disease in a human or non-human subject. The disclosure herein can be used to treat a chronic inflammatory skin disease in a human or non-human subject. The disclosure herein can be used to treat eczema in a human or non-human subject. The disclosure herein can be used to treat rosacea in a human or non-human subject. The disclosure herein can be used to treat psoriasis in a human or non-human subject.

The disclosure herein can be used to treat seborrheic dermatitis in a human or non-human subject. The disclosure herein can be used to treat allergic contact dermatitis in a human or non-human subject. The disclosure herein can be used to treat ichthyosis disease in a human or non-human subject. The disclosure herein can be used to treat NS in a human or non-human subject.

In some embodiments, the oligonucleotide (e.g., synthetic oligonucleotide) and/or SNA is administered in a pharmaceutical composition. Routes of administration include but are not limited to cutaneous, subcutaneous, nodal, systemic, intravenous, intrathecal, intracranial, oral, parenteral, intracranial, intramuscular, intranasal, sublingual, intratracheal, inhalation, ocular, vaginal, and rectal.

In some embodiments, the oligonucleotide (e.g., synthetic oligonucleotide), SNA, or composition thereof (e.g., pharmaceutical composition thereof) is administered topically (e.g., cutaneously).

As disclosed herein, the dose (e.g., an effective amount) as it relates to administration of an oligonucleotide (e.g., synthetic oligonucleotide) and/or an SNA disclosed herein (e.g., without limitation the phrases “an effective amount of a synthetic oligonucleotide,” “an effective amount of a pharmaceutical composition,” “an effective amount of a spherical nucleic acid”, etc.) refers to the total weight or total mass of active agent (e.g., total weight or total mass of the synthetic oligonucleotides) in the SNA administered to the subject (e.g., subject with a disease or disorder). As disclosed herein, an “effective amount” refers to an amount that is capable of improving one or more symptoms of a disease or disorder or an amount that is capable of improving a metric associated with a disease or disorder.

In some embodiments, a dose disclosed herein is considered a fixed dose or a discrete dose. In some embodiments, a dose disclosed herein can be adjusted to depend on body weight or be made dependent on body weight. A non-limiting example includes a dose of 2 mg of an oligonucleotide (e.g., synthetic oligonucleotide), an SNA, or a composition thereof (e.g., pharmaceutical composition thereof) disclosed herein, that can also be administered as 2 mg/kg body weight, which depends on kg of body weight. In some embodiments, a dose disclosed herein is independent of body weight. In some embodiments, a dose disclosed herein can be adjusted to depend on body surface area (e.g., total body surface area, skin surface area to be treated, etc.) or be made dependent on body surface area. A non-limiting example includes a dose of 2 mg of an oligonucleotide (e.g., synthetic oligonucleotide), an SNA, or a composition thereof (e.g., pharmaceutical composition thereof) disclosed herein, that can also be administered as 2 mg/m² body surface area, which depends on m² of body surface area (e.g., total body surface area, skin surface area to be treated, etc.). In some embodiments, a dose disclosed herein is independent of body surface area.

In some embodiments, an oligonucleotide (e.g., synthetic oligonucleotide), an SNA, or a composition thereof (e.g., pharmaceutical composition thereof) disclosed herein is administered once a day, once every three days, once a week, once every two weeks, once every three weeks, once every four weeks, once every five weeks, once every six weeks, once every seven weeks, once every eight weeks, once every nice weeks, once every 10 weeks, once every 12 weeks, once every 18 weeks, once every 24 weeks, once a month, once every two months, once every three months, once every four months, once every five months, once every six months, once every seven months, once every eight months, once every nine months, once every 10 months, once every 11 months, once a year, once every two years, once every three years, once every four years.

In some embodiments, an oligonucleotide (e.g., synthetic oligonucleotide), a SNA, or a composition thereof (e.g., pharmaceutical composition thereof) disclosed herein is administered to a subject once a week, twice a week or three times per week, for four weeks, six weeks, eight weeks, 10 weeks, 12 weeks, 14 weeks, 16 weeks, 18 weeks, 20 weeks, 24 weeks, one month, two months, three months, four months, five months, six months, seven months, eight months, nine months, 10 months, 11 months, one year, two years, three years, four years, five years, six years.

In some embodiments an oligonucleotide (e.g., synthetic oligonucleotide), a SNA, or a composition thereof (e.g., pharmaceutical composition thereof) disclosed herein is administered to a subject every three weeks for four weeks, six weeks, eight weeks, 10 weeks, 12 weeks, 14 weeks, 16 weeks, 18 weeks, 20 weeks, 24 weeks, one month, two months, three months, four months, five months, six months, seven months, eight months, nine months, 10 months, 11 months, one year, two years, three years, four years, five years, six years, seven years, eight years, nine years, or 10 years. In some embodiments, the SNA is administered every three weeks. In some embodiments, the SNA is administered about or at least about every four weeks, five weeks, six weeks, 2 months, three months, six months, nine months, one year, 1.5 years, two years, 2.5 years, three years, 3.5 years, four years, 4.5 years, five years, 5.5 years, or six years.

In some embodiments, the duration of the method for treating a disease or disorder (e.g., NS) with an oligonucleotide (e.g., synthetic oligonucleotide), a SNA, or a composition thereof (e.g., pharmaceutical composition thereof) disclosed herein is for about or at least 3 months, for about or at least six months, for about or at least nine months, for about or at least one year, for about or at least 1.5 years, for about or at least two years, for about or at least 2.5 years, for about or at least 3 years, for about or at least 3.5 years, for about or at least 4 years, for about or at least 4.5 years, for about or at least 5 years, for about or at least 5.5 years, for about or at least 6 years, for about or at least 6.5 years, for about or at least 7 years, for about or at least 7.5 years, for about or at least 8 years, for about or at least 8.5 years, for about or at least 9 years, for about or at least 9.5 years, for about or at least 10 years, for about or at least 15 years, for about or at least 20 years or more than 20 years, or for the lifetime of the subject.

In some embodiments, an effective amount is from about 0.1 μg to 10,000 mg per dose, at least about 1 μg to 8,000 mg per dose, or 10 μg to 100 μg per dose. In some embodiments the dose administered is about or at least about 1 μgμg, about or at least about 5 μg, about or at least about 10 μg, about or at least about 50 μg, about or at least about 100 μg, about or at least about 200 μg, about or at least about 350 μg, about or at least about 500 μg, about or at least about 1 mg, about or at least about 5 mg, about or at least about 10 mg, about or at least about 50 mg, about or at least about 100 mg, about or at least about 200 mg, about or at least about 350 mg, about or at least about 500 mg, about or at least about 1000 mg or more per dose, and any range or combination thereof. In non-limiting examples of a derivable range from the doses disclosed herein, a range of about 5 mg to about 100 mg, about 5 μg to about 500 mg, etc., can be administered based on the doses disclosed herein.

Stated in terms of subject body weight, in some embodiments the dose administered is about or at least about 1 μg/kg of body weight, about or at least about 5 μg/kg of body weight, about or at least about 10 μg/kg of body weight, about or at least about 50 μg/kg of body weight, about or at least about 100 μg/kg of body weight, about or at least about 200 μg/kg of body weight, about or at least about 350 μg/kg of body weight, about or at least about 500 μg/kg of body weight, about or at least about 1 mg/kg of body weight, about or at least about 5 mg/kg of body weight, about or at least about 10 mg/kg of body weight, about or at least about 50 mg/kg of body weight, about or at least about 100 mg/kg of body weight, about or at least about 200 mg/kg of body weight, about or at least about 350 mg/kg of body weight, about or at least about 500 mg/kg of body weight, to about or at least about 1000 mg/kg of body weight or more per administration, and any range or combination thereof. In non-limiting examples of a derivable range from the numbers listed herein, a range of about 5 mg/kg of body weight to about 100 mg/kg of body weight, about 5 μg/kg of body weight to about 500 mg/kg of body weight, etc., can be administered, based on the numbers disclosed above.

The absolute amount (e.g., a discrete dose) will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment.

In some embodiments, an oligonucleotide (e.g., a synthetic oligonucleotide), an SNA, or composition thereof (e.g., pharmaceutical composition thereof) disclosed herein is administered at a dose between 0.1 mg and 10 mg, between 0.2 mg and 10 mg, between 0.3 mg and 10 mg, between 0.4 mg and 10 mg, between 0.5 mg and 10 mg, between 0.6 mg and 10 mg, between 0.7 mg and 10 mg, between 0.8 mg and 10 mg, between 0.9 mg and 10 mg, between 1 mg and 10 mg, between 1 mg and 1,000 mg, between 1 mg and 900 mg, between 1 mg and 800 mg, between 1 mg and 700 mg, between 1 mg and 600 mg, between 1 mg and 500 mg, between 1 mg and 450 mg, between 1 mg and 400 mg, between 1 mg and 350 mg, between 1 mg and 300 mg, between 1 mg and 250 mg, between 1 mg and 200 mg, between 1 mg and 150 mg, between 1 mg and 100 mg, between 1 mg and 90 mg, between 1 mg and 80 mg, between 1 mg and 70 mg, between 1 mg and 60 mg, between 1 mg and 60 mg, between 1 mg and 50 mg, between 1 mg and 49 mg, between 1 mg and 48 mg, between 1 mg and 47 mg, between 1 mg and 46 mg, between 1 mg and 45 mg, between 1 mg and 44 mg, between 1 mg and 43 mg, between 1 mg and 42 mg, between 1 mg and 41 mg, between 1 mg and 40 mg, between 1 mg and 39 mg, between 1 mg and 38 mg, between 1 mg and 37 mg, between 1 mg and 36 mg, between 1 mg and 35 mg, between 1 mg and 34 mg, between 1 mg and 33 mg, between 1 mg and 32 mg, between 1 mg and 31 mg, between 1 mg and 30 mg, between 1 mg and 29 mg, between 1 mg in 28 mg, between 1 mg and 27 mg, between 1 mg and 26 mg, between 1 mg and 25 mg, between 1 mg and 24 mg, between 1 mg and 23 mg, between 1 mg and 22 mg, between 1 mg and 21 mg, between 1 mg and 20 mg, between 1 mg and 19 mg, between 1 mg and 18 mg, between 1 mg and 17 mg, between 1 mg and 16 mg, between 1 mg and 15 mg, between 1 mg and 14 mg, between 1 mg and 13 mg, between 1 mg and 12 mg, between 1 mg and 11 mg, between 1 mg and 10 mg, between 1 mg and 9 mg, between 1 mg and 8 mg, between 1 mg and 7 mg, between 1 mg and 6 mg, between 1 mg and 5 mg, between 1 mg and 4 mg, between 1 mg and 2 mg, between 1 mg and 1.5 mg, between 1 mg and 3 mg, between 3 mg and 5 mg, between 5 mg and 7 mg, between 7 mg and 9 mg, between 9 mg and 14 mg, between 15 mg and 17 mg, between 18 mg and 31 mg, between 31 mg and 33 mg, between 0.5 mg and 2 mg, between 2 mg and 4 mg, between 11 mg and 13 mg, between 23 mg and 25 mg, between 2 mg and 31 mg, between 2 mg and 30 mg, between 2 mg and 29 mg, between 2 mg and 28 mg, between 2 mg and 27 mg, between 2 mg and 26 mg, between 2 mg and 25 mg, between 2 mg and 24 mg, between 2 mg and 23 mg, between 2 mg and 22 mg, between 2 mg and 21 mg, between 2 mg and 20 mg, between 2 mg and 19 mg, between 2 mg and 18 mg, between 2 mg and 17 mg, between 2 mg and 16 mg, between 2 mg and 15 mg, between 2 mg and 14 mg, between 2 mg and 13 mg, between 2 mg and 12 mg, between 2 mg and 11 mg, between 2 mg and 10 mg, between 2 mg and 9 mg, between 2 mg and 8 mg, between 2 mg and 7 mg, between 2 mg and 6 mg, between 2 mg and 5 mg, between 2 mg and 3 mg.

In some embodiments, an oligonucleotide (e.g., a synthetic oligonucleotide), an SNA, or composition thereof (e.g., pharmaceutical composition thereof) disclosed herein is administered at a dose of or about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 30 mg, 13.5 mg, 40 mg, 14.5 mg, 50 mg, 15.5 mg, 60 mg, 16.5 mg, 70 mg, 70.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 50 mg, 400 mg, 450 mg, 500 mg, 550 mg, 600 mg, six and 50 mg, 700 mg, 750 mg, 800 mg, 850 mg, 900 mg, 950 mg, or 1000 mg, or any range or combination thereof.

In some embodiments, an oligonucleotide (e.g., a synthetic oligonucleotide), an SNA, or composition thereof (e.g., pharmaceutical composition thereof) disclosed herein is administered at a dose of at least or at least about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 30 mg, 13.5 mg, 40 mg, 14.5 mg, 50 mg, 15.5 mg, 60 mg, 16.5 mg, 70 mg, 70.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 50 mg, 400 mg, 450 mg, 500 mg, 550 mg, 600 mg, 650 mg, 700 mg, 750 mg, 800 mg, 850 mg, 900 mg, 950 mg, or 1000 mg, or any range or combination thereof.

In some embodiments, an oligonucleotide (e.g., a synthetic oligonucleotide), an SNA, or composition thereof (e.g., pharmaceutical composition) disclosed herein is administered at a dose greater than or greater than about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 30 mg, 13.5 mg, 40 mg, 14.5 mg, 50 mg, 15.5 mg, 60 mg, 16.5 mg, 70 mg, 70.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 50 mg, 400 mg, 450 mg, 500 mg, 550 mg, 600 mg, 650 mg, 700 mg, 750 mg, 800 mg, 850 mg, 900 mg, 950 mg, or 1000 mg, or any range or combination thereof.

As disclosed herein, a “reference level,” refers to a corresponding level in a subject with a disease or disorder (e.g., a subject with NS) who has been administered a linear oligonucleotide (e.g., a synthetic oligonucleotide disclosed herein) which is not in an SNA format or pharmaceutical composition thereof; to a corresponding level in a subject with a disease or disorder (e.g., NS) who has not been administered an SNA (e.g., an SNA disclosed herein) or pharmaceutical composition thereof; to a corresponding level in a subject with a disease or disorder (e.g., NS) who has not been administered a linear oligonucleotide (e.g., synthetic oligonucleotide) disclosed herein; to a corresponding level in a subject with the disease or disorder (e.g., NS) prior to administration of an oligonucleotide (e.g., synthetic oligonucleotide), SNA or composition thereof (e.g., pharmaceutical composition thereof) disclosed herein to the subject with the disease or disorder (e.g., NS); to a corresponding level in a subject with a disease or disorder (e.g., NS) who has been administered a treatment other than a synthetic oligonucleotide, SNA, or composition thereof (e.g., pharmaceutical composition thereof) disclosed herein; to a corresponding level in a subject without NS; or to a corresponding level in a subject without a disease or disorder, such as an ichthyosis disease or disorder.

In some embodiments, administration of an oligonucleotide (e.g., synthetic oligonucleotide), an SNA, or a composition thereof (e.g., pharmaceutical composition thereof) to a subject decreases KLK mRNA levels (e.g., KLK5 mRNA levels or KLK7 mRNA levels) or KLK protein levels (e.g., KLK5 protein levels or KLK7 protein levels). In some embodiments, the level is decreased in the subject by at least or about 5%, at least or about 10%, at least or about 15%, at least or about 16%, at least or about 17%, at least or about 18%, at least or about 19%, at least or about 20%, at least or about 21%, at least or about 22%, at least or about 23%, at least or about 24%, at least or about 25%, at least or about 26%, at least or about 27%, at least or about 28%, at least or about 29%, at least or about 30%, at least or about 31%, at least or about 32%, at least or about 33%, at least or about 34%, at least or about 35%, at least or about 36%, at least or about 37%, at least or about 38%, at least or about 39%, at least or about 40%, at least or about 41%, at least or about 42%, at least or about 43%, at least or about 44%, at least or about 45%, at least or about 46%, at least or about 47%, at least or about 48%, at least or about 49%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97%, at least or about 98%, at least or about 99%, or about 100%, or any range or combination thereof, relative to a reference level.

In some embodiments, the KLK mRNA levels (e.g., KLK5 mRNA levels or KLK7 mRNA levels) or KLK protein levels (e.g., KLK5 protein levels or KLK7 protein levels) are decreased in a cell or cells of a subject with a disease or disorder (e.g., NS). In some embodiments, the KLK mRNA levels (e.g., KLK5 mRNA levels or KLK7 mRNA levels) or KLK protein levels (e.g., KLK5 protein levels or KLK7 protein levels) are decreased in a tissue or tissues of a subject with a disease or disorder (e.g., NS). In some embodiments, the KLK mRNA levels (e.g., KLK5 mRNA levels or KLK7 mRNA levels) or KLK protein levels (e.g., KLK5 protein levels or KLK7 protein levels) are decreased in an organ or organs of a subject with a disease or disorder (e.g., NS). In some embodiments, the KLK mRNA levels (e.g., KLK5 mRNA levels or KLK7 mRNA levels) or KLK protein levels (e.g., KLK5 protein levels or KLK7 protein levels) are decreased in the skin of a subject with a disease or disorder (e.g., NS).

In some embodiments, an oligonucleotide (e.g., synthetic oligonucleotide), a SNA, or a composition thereof (e.g., pharmaceutical composition thereof) is contacted with a cell to decrease KLK mRNA levels (e.g., KLK5 mRNA levels or KLK7 mRNA levels) or KLK protein levels (e.g., KLK5 protein levels or KLK7 protein levels) in the cell. In some embodiments, the level in the cell is decreased by at least or about 5%, at least or about 10%, at least or about 15%, at least or about 16%, at least or about 17%, at least or about 18%, at least or about 19%, at least or about 20%, at least or about 21%, at least or about 22%, at least or about 23%, at least or about 24%, at least or about 25%, at least or about 26%, at least or about 27%, at least or about 28%, at least or about 29%, at least or about 30%, at least or about 31%, at least or about 32%, at least or about 33%, at least or about 34%, at least or about 35%, at least or about 36%, at least or about 370, at least or about 38%, at least or about 39%, at least or about 40%, at least or about 41%, at least or about 42%, at least or about 43%, at least or about 44%, at least or about 45%, at least or about 46%, at least or about 47%, at least or about 48%, at least or about 49%, at least or about 50%, at least or about 55%, at least or about 60%, at least or about 65%, at least or about 70%, at least or about 75%, at least or about 80%, at least or about 85%, at least or about 90%/6, at least or about 95%, at least or about 96%, at least or about 97%, at least or about 98%, at least or about 99%, or about 100% relative to a reference level.

In some embodiments, an oligonucleotide (e.g., synthetic oligonucleotide), a SNA, or a composition thereof (e.g., pharmaceutical composition thereof) is administered to a subject at a dose which results in one or more of decreased KLK mRNA expression, decreased KLK mRNA translation, and/or decreased KLK protein expression (e.g., decreased KLK5 or KLK7 mRNA expression, decreased KLK5 or KLK7 pre-mRNA expression, decreased KLK5 or KLK7 mRNA translation, and/or decreased KLK5 or KLK7 protein expression) by at least or about 5%, at least or about 10%, at least or about 15%, at least or about 16%, at least or about 17%, at least or about 18%, at least or about 19%, at least or about 20%, at least or about 21%, at least or about 22%, at least or about 23%, at least or about 24%, at least or about 25%, at least or about 26%, at least or about 27%, at least or about 28%, at least or about 29%, at least or about 30%, at least or about 31%, at least or about 32%, at least or about 33%, at least or about 34%, at least or about 35%, at least or about 36%, at least or about 37%, at least or about 38%, at least or about 39%, at least or about 40%, at least or about 41%, at least or about 42%, at least or about 43%, at least or about 44%, at least or about 45%, at least or about 46%, at least or about 47%, at least or about 48%, at least or about 49%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97%, at least or about 98%, at least or about 99%, or about 100% relative to a reference level.

In some embodiments, the KLK mRNA expression (e.g., KLK5 and/or KLK7 mRNA expression), KLK pre-mRNA expression (e.g., KLK5 and/or KLK7 pre-mRNA expression), KLK mRNA translation (e.g., KLK5 and/or KLK7 mRNA translation), and/or KLK protein expression (e.g., KLK5 and/or KLK7 protein expression) are decreased in a cell or cells of a subject. In some embodiments, the KLK mRNA expression (e.g., KLK5 and/or KLK7 mRNA expression), KLK pre-mRNA expression (e.g., KLK5 and/or KLK7 pre-mRNA expression), KLK mRNA translation (e.g., KLK5 and/or KLK7 mRNA translation), and/or KLK protein expression (e.g., KLK5 and/or KLK7 protein expression) are decreased in a tissue or tissues of a subject. In some embodiments, the KLK mRNA expression (e.g., KLK5 and/or KLK7 mRNA expression), KLK pre-mRNA expression (e.g., KLK5 and/or KLK7 pre-mRNA expression), KLK mRNA translation (e.g., KLK5 and/or KLK7 mRNA translation), and/or KLK protein expression (e.g., KLK5 and/or KLK7 protein expression) are decreased in the skin of a subject. In some embodiments, the KLK mRNA expression (e.g., KLK5 and/or KLK7 mRNA expression), KLK pre-mRNA expression (e.g., KLK5 and/or KLK7 pre-mRNA expression), KLK mRNA translation (e.g., KLK5 and/or KLK7 mRNA translation), and/or KLK protein expression (e.g., KLK5 and/or KLK7 protein expression) are decreased in an organ or organs of a subject (e.g., subject with NS).

As disclosed herein, “measuring kallikrein-related peptidase (KLK) mRNA levels” or “measuring kallikrein-related peptidase (KLK) protein levels” refers to quantifying or qualitatively evaluating the amount of KLK mRNA (e.g., KLK5 and/or KLK7 mRNA) or KLK protein (e.g., KLK5 and/or KLK7 protein) in a given sample. In some embodiments, measuring KLK mRNA levels (e.g., KLK5 and/or KLK7 mRNA levels) may refer to using laboratory methods known to those of ordinary skill in the art to quantify or qualitatively evaluate the amount of KLK mRNA (e.g., KLK5 and/or KLK7 mRNA) in a given sample, using methods including but not limited to quantitative real-time reverse transcription polymerase chain reaction (qRT-PCR), Northern blotting, in situ hybridization, or spectrophotometric analysis. In some embodiments, measuring KLK protein levels (e.g., KLK5 and/or KLK7 protein levels) may refer to using laboratory methods known to those of ordinary skill in the art to quantify or qualitatively evaluate the amount of KLK protein (e.g., KLK5 and/or KLK7 protein) in a given sample, using methods including but not limited to Western blotting, enzyme-linked immunosorbent assays (ELISA), immunohistochemical staining, immunofluorescent staining, mass spectrometry, spectrophotometric analysis, or colorimetric analysis such as bicinchoninic acid (BCA) assays.

As disclosed herein, “a value that is less than” and equivalent phrases refer to values which are decreased or quantified to be smaller than a value or values to which they are compared.

As disclosed herein, “pharmaceutically acceptable salts” are physiologically and pharmaceutically acceptable salts of the nucleic acids (e.g., salts that retain the desired biological activity of the compound of interest and do not impart undesired toxicological effects thereto). Pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. Pharmaceutical compositions of the present disclosure comprise an effective amount of one or more agents, dissolved or dispersed in a pharmaceutically acceptable carrier.

Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In some embodiments, a “pharmaceutical or pharmacologically acceptable” composition refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans. This term requires that a compound or composition be nontoxic and sufficiently pure so that no further manipulation of the compound or composition is needed prior to administration to humans.

In certain embodiments, pharmaceutical compositions may comprise, for example, at least about 0.000001% (w/w) of an active agent (e.g., an oligonucleotide, such as a synthetic oligonucleotide, or an SNA disclosed herein). In other embodiments, the active compound may comprise between about 2% to about 75% of the weight of the unit (w/w), or between about 25% to about 60%, for example, and any range or combination thereof. In some embodiments, the active agent (e.g., an oligonucleotide, such as a synthetic oligonucleotide, or an SNA disclosed herein) disclosed herein comprises between 0.000001% and 0.00001%, between 0.00001% and 0.00010%, between 0.0001% and 0.0010%, between 0.001% and 0.010%, between 0.01% and 0.1%, between 0.1% and 1%, between 1% and 5%, between 5% and 10%, between 10% and 15%, between 15% and 20%, between 20% and 25%, between 25% and 30%, between 30% and 40%, between 40% and 50% (w/w), and any range or combination thereof. In some embodiments, the active agent (e.g., oligonucleotide, such as a synthetic oligonucleotide or an SNA disclosed herein) comprises 0.00007%, 0.007%, 0.01%, 0.1%, 1% (w/w).

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. The agent may comprise different types of carriers depending on whether it is to be administered in solid, liquid, gel, cream, or aerosol form, and whether it need to be sterile for such routes of administration as injection.

An oligonucleotide (e.g., synthetic oligonucleotide), SNA, or compositions thereof (e.g., pharmaceutical composition thereof) disclosed herein can be administered intravenously, intradermally, intraarterially, intralesionally, intratumorally, intracranially, intrathecally, intraarticularly, intraprostatically, intrapleurally, intratracheally, intranasally, intravitreally, intravaginally, intrarectally, topically, intramuscularly, intraperitoneally, subcutaneously, subconjunctival, intravascularly, mucosally, intrapericardially, intraumbilically, intraocularly, via eyedrops, orally, topically (e.g., cutaneously), locally, via inhalation (e.g., aerosol inhalation), via injection, via infusion, via continuous infusion, via localized perfusion bathing target cells directly, via a catheter, or via a lavage. In some embodiments, an oligonucleotide (e.g., synthetic oligonucleotide), SNA, or compositions thereof (e.g., pharmaceutical composition thereof) disclosed herein can be administered intradermally.

In some embodiments, an oligonucleotide (e.g., synthetic oligonucleotide), SNA, or compositions thereof (e.g., pharmaceutical composition thereof) disclosed herein can be administered in creams, in gels, in lipid compositions (e.g., liposomes), or by other method or any combination of the forgoing as would be known to one of ordinary skill in the art.

In some embodiments, an oligonucleotide (e.g., synthetic oligonucleotide), an SNA, or compositions thereof (e.g., pharmaceutical composition thereof) disclosed herein is administered topically (e.g., cutaneously).

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The agent, which may be an oligonucleotide (e.g., synthetic oligonucleotide), SNA, or compositions thereof (e.g., pharmaceutical composition thereof) as disclosed, may be formulated into a composition in a free base, neutral or salt form.

An oligonucleotide (e.g., synthetic oligonucleotide), SNA, or compositions thereof (e.g., pharmaceutical composition thereof) disclosed herein may be administered directly to a tissue. Direct tissue administration may be achieved by direct injection, topical application, or local application. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The formulations are administered in pharmaceutically acceptable compositions, which may routinely contain pharmaceutically acceptable concentrations of salt, buffering agents, preservatives, compatible carriers, adjuvants, excipients, and optionally other therapeutic ingredients. A composition disclosed herein (e.g., synthetic oligonucleotide, SNA, etc.) may be administered in a pharmaceutical composition. In general, a pharmaceutical composition comprises the composition and a pharmaceutically-acceptable carrier. As used herein, a pharmaceutically-acceptable carrier means a non-toxic material that does not interfere with the effectiveness of the biological activity of the active ingredients. Pharmaceutically acceptable carriers include, without limitation, diluents, fillers, salts, buffers, stabilizers, solubilizers and other materials which are well-known in the art. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the disclosure. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

A composition disclosed herein (e.g., oligonucleotide, synthetic oligonucleotide, SNA, etc.) may be formulated into preparations in solid, semi-solid, liquid or gaseous forms such as tablets, capsules, powders, granules, ointments, solutions, depositories, inhalants and injections, and usual ways for oral, parenteral or surgical administration. The disclosure also embraces pharmaceutical compositions which are formulated for local administration, such as by implants.

A composition disclosed herein (e.g., synthetic oligonucleotide, SNA, etc.), when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In some embodiments, a delivery vehicle is a biocompatible microparticle or implant that is suitable for implantation into the mammalian recipient. Other delivery systems can include time-release, delayed release or sustained release delivery systems. Such systems can avoid repeated administrations of the compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides.

This invention is not limited in its application to the details of construction and the arrangement of components set forth in the following description. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Those skilled in the art will recognize, or be able to ascertain using no more than routine experimentation, many equivalents to the specific embodiments of the invention disclosed herein. Such equivalents are intended to be encompassed by the following claims.

All references, including patent documents, disclosed herein are incorporated by reference in their entirety.

### EXAMPLES

### Example 1. Nucleic Acid (not in Spherical Nucleic Acid (SNA) Format) Compounds Targeted to Human KLK5 Gene

About nineteen hundred newly designed nucleic acid compounds (not in SNA format) comprising antisense oligonucleotides targeting the human KLK5 gene sequence were tested for their effect on human KLK5 mRNA in vitro in A431 cells. Table 1 lists the approximately fifteen hundred gapmer antisense oligonucleotides and approximately four hundred fully 2′-O-methyl modified antisense oligonucleotides that were used. All of the approximately nineteen hundred antisense oligonucleotides also contained two internal spacer 18 (iSp18) modifications and a terminal cholesterol-TEG moiety (3CholTEG).

A431 cells were plated into 96-well tissue culture plates at a density of 15,000 cells per well. Lipidated nucleic acid compounds (not in SNA format) were diluted in PBS to give a final concentration of 20 μM. Prior to treatment, cell culture media was replaced with 90 μL of fresh media and 10 μL of compound was added to each well to yield a final treatment concentration of 2 μM. Appropriate controls, including transfected siRNAs and nonsense control lipidated nucleic acid compounds (not in SNA format), were also added to each treatment plate. After a treatment period of approximately 48 hours, RNA was isolated from the cells and KLK5 mRNA levels were measured by quantitative reverse-transcriptase polymerase chain reaction (qRT-PCR). Relative KLK5 expression in each sample was calculated using the 2^{-ΔΔCt} method.

The results are shown in Table 1 in the “% inhib.” columns. Approximately seventy-two compounds with greater than 30% inhibition were identified. The best-performing compounds are shown in Table 3.

### Example 2. Nucleic Acid (not in SNA Format) Compounds Targeted to Human KLK7Gene

About twenty-two hundred newly designed nucleic acid (not in SNA format) compounds comprising antisense oligonucleotides targeting the human KLK7 gene sequence were tested for their effect on human KLK7 mRNA in vitro in A431 cells. Table 2 lists the approximately nineteen hundred gapmer antisense oligonucleotides and approximately three hundred fully 2′-O-methyl modified antisense oligonucleotides that were used. All of the approximately twenty-two hundred antisense oligonucleotides also contained two internal spacer 18 (iSp18) modifications and a terminal cholesterol-TEG moiety (3CholTEG).

A431 cells were plated into 96-well tissue culture plates at a density of 15,000 cells per well. Lipidated nucleic acid compounds (not in SNA format) were diluted in PBS to give a final concentration of 20 μM. Prior to treatment, cell culture media was replaced with 90 μL of fresh media and 10 μL of compound was added to each well to yield a final treatment concentration of 2 μM. Appropriate controls, including transfected siRNAs and nonsense control lipidated nucleic acid compounds (not in SNA format), were also added to each treatment plate. After a treatment period of approximately 48 hours, RNA was isolated from the cells and KLK7 mRNA levels were measured by quantitative reverse-transcriptase polymerase chain reaction (qRT-PCR). Relative KLK7 expression in each sample was calculated using the 2^{-ΔΔCt} method.

The results are shown in Table 2 under “% inhibition” column. Approximately forty-two compounds with greater than 30% inhibition were identified. The best-performing compounds are shown in Table 4.

### Example 3. Spherical Nucleic Acid (SNA) Compounds Targeted to Human KLK5 and KLK7

Newly designed SNA compounds and lipidated nucleic acid compounds comprising antisense oligonucleotides targeting the human KLK5 and KLK7 gene sequences were tested for their effect on human KLK5 and KLK7 mRNA in vitro in A431 and HaCaT cells. Each of the antisense oligonucleotides contained various modifications, including base modifications, sugar modifications, and internucleoside linkage modifications. Each nucleic oligonucleotide also comprised a cholesterol-TEG or tocopherol lipid moiety at its 5′- or 3′-end. To formulate SNAs comprising the oligonucleotides, liposomes were first prepared from dried DOPC (1,2-dioleoyl-sn-glycero-3-phosphocholine), rehydrated in PBS by agitating overnight, and homogenized under pressure until a mean diameter of 18-20 nm and a polydispersity index of <0.2 were reached. SNAs were then formed under sterile conditions by mixing the lipidated oligonucleotides with liposomes at a 50 to 1 w/w ratio of lipid to oligonucleotide.

To test the effects of SNAs and lipidated nucleic acid compounds on KLK5 and KLK7 gene expression, A431 and HaCaT cells were treated and mRNA was quantified by quantitative reverse transcription polymerase chain reaction (qRT-PCR). A431 cells were cultured using protocols developed by ATCC. HaCaT cells were cultured similarly. Cells were plated into 96-well tissue culture plates at a density of 15K cells per well at 16 hours prior to treatment. SNAs and lipidated compounds were diluted in PBS to give a final concentration of 20 μM. Prior to treatment cell culture media was replaced with 90 μL of fresh media and 10 μL of compound was added to each well to give a final treatment concentration of 2 μM. Appropriate controls including transfected siRNAs and nonsense control SNAs were also added to each treatment plate.

Cells were lysed 48-hours post-treatment using the Cells-to-Ct method according to manufacturer instructions. Lysates were immediately frozen at −80° C. and stored for at least 16 hours before processing. qRT-PCR was performed using TaqMan assays set up as follows: 2 μl of cell lysate plus 8 μl of qRT-PCR Master Mix multiplexed with FAM-labeled KLK5 and KLK7 probes and custom VIC-labeled GAPDH probes. Table 23 lists the probes used for qRT-PCR analysis. Technical duplicates for each treatment were run on a QuantStudio 12K PCR system using manufacturer-recommended thermocycler settings. KLK cycle threshold (Ct) values were first normalized to GAPDH expression by calculating the ΔCt values. Changes in KLK mRNA expression were then determined by normalizing each treatment group to either the median gene expression across the plate or the average of untreated wells using the 2^{-ΔΔCt} method.

TABLE 23
Probes used for gene expression measurement assays.

| | | Gene Expression ID | | |
|---|---|---|---|---|
| Target | Manufacturer | (ThermoFisher) | 5′DYE | 3′Quencher |
| KLK5 | ThermoFisher Scientific | Hs01548152_g1 | FAM | MGB |
| KLK5 | ThermoFisher Scientific | Hs01548153_m1 | FAM | MGB |
| KLK7 | ThermoFisher Scientific | Hs01012732_g1 | FAM | MGB |
| KLK7 | ThermoFisher Scientific | Hs00192503_m1 | FAM | MGB |
| GAPDH | Custom | — | VIC | QSY |

| Name | Sequence (5′-3′) |
|---|---|
| GAPDH-FWD | CAAGGTCATCCATGACAACTTTG (SEQ ID NO: 7848) |
| GAPDH-REV | GGGCCATCCACAGTCTTCT (SEQ ID NO: 7808) |
| GAPDH-Probe | VIC-ACCACAGTCCATGCCATCACTGCCA-QSY (SEQ ID NO: 7849) |

Tables 5-22 show sequence information and results of gene expression analysis. Table 5 lists 2′-O-methyl modified lipidated nucleic acid compounds targeted to KLK5 and shows the effects of the lipidated nucleic acid compound treatment on KLK5 gene expression in A431 cells.

The compounds listed in Table 5 can also be used in SNA format, by preparing SNAs as described above. Tables 7, 8, 10 list SNA compounds comprising oligonucleotides targeted to KLK5 and show the effects of the SNA treatment on KLK5 gene expression in A431 cells. Tables 6, 9 and 11 list SNA compounds comprising oligonucleotides targeted to KLK7 and show the effects of the SNA treatment on KLK7 gene expression in A431 cells.

Table 12 lists bispecific SNA compounds comprising two populations of oligonucleotides, a first targeted to KLK5 and a second targeted to KLK7. Table 13 lists alternate bispecific SNA compounds, and shows the effects of those SNAs on KLK5 and KLK7 gene expression in A431 cells. Table 14 lists two preferred bispecific SNA compounds and shows the effects of those SNAs on KLK5 and KLK7 gene expression in A431 cells. Tables 15 and 17 show KLK5 gene expression results measured in A431 cells following treatment with bispecific SNAs. Table 16 shows average KLK5 gene expression and 1C50 values for the inhibition of KLK5 gene expression by bispecific SNAs listed in Table 15. Table 18 shows KLK5 gene expression results measured in HaCaT cells following treatment with bispecific SNAs.

Tables 19 and 21 show KLK7 gene expression results measured in A431 cells following treatment with bispecific SNAs. Table 20 shows average KLK7 gene expression and IC50 values for the inhibition of KLK7 gene expression by bispecific SNAs listed in Table 19. Table 22 shows KLK7 gene expression results measured in HaCaT cells following treatment with bispecific SNAs.

### Other Embodiments

Embodiment 1. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmUmGmAmGC*C*A*C*C*C*T*C*A*C*mCmAmGmGmU/iSp18//iSp18//3CholTEG/ (SEQ ID NO: 99), or a pharmaceutically acceptable salt thereof.

Embodiment 2. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmCmCmCmUG*C*C*T*G*C*T*G*A*G*mCmCmAmCmC/iSp18//iSp18//3CholTEG/ (SEQ ID NO: 109), or a pharmaceutically acceptable salt thereof.

Embodiment 3. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmCmUmUmCC*C*T*G*C*C*T*G*C*T*mGmAmGmCmC/iSp18//iSp18//3ChoTEG/ (SEQ ID NO: 112), or a pharmaceutically acceptable salt thereof.

Embodiment 4. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmCmCmUmUC*C*C*T*G*C*C*T*G*C*mUmGmAmGmC/iSp18//iSp18//3CholTEG/ (SEQ ID NO: 113), or a pharmaceutically acceptable salt thereof.

Embodiment 5. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmUmCmCmUT*C*C*C*T*G*C*C*T*G*mCmUmGmAmG/iSp18//iSp18//3CholTEG/ (SEQ ID NO: 114), or a pharmaceutically acceptable salt thereof.

Embodiment 6. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmCmCmAmCA*T*C*C*A*G*G*G*G*G*mGmUmCmUmU/iSp18//iSp18//3CholTEG/ (SEQ ID NO: 315), or a pharmaceutically acceptable salt thereof.

Embodiment 7. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmUmAmGmAG*G*G*G*T*G*G*T*C*A*mCmAmGmGmA/iSp18//iSp18//3CholTEG/ (SEQ ID NO: 403), or a pharmaceutically acceptable salt thereof.

Embodiment 8. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmAmGmGmUC*C*T*G*G*T*T*G*C*T*mCmCmCmAmG/iSp18//iSp18//3CholTEG/ (SEQ ID NO: 434), or a pharmaceutically acceptable salt thereof.

Embodiment 9. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmCmGmAmCC*G*G*G*C*G*T*C*T*T*mCmCmCmCmG/iSp18//iSp18//3CholTEG/ (SEQ ID NO: 465), or a pharmaceutically acceptable salt thereof.

Embodiment 10. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmGmGmUmGT*G*C*A*T*A*T*C*G*C*mAmGmUmCmG/iSp18//iSp18//3CholTEG/ (SEQ ID NO: 516), or a pharmaceutically acceptable salt thereof.

Embodiment 11. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmAmGmCmAG*C*C*A*C*T*G*T*G*G*mAmUmGmCmA/iSp18//iSp18//3CholTEG/ (SEQ ID NO: 596), or a pharmaceutically acceptable salt thereof.

Embodiment 12. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmCmUmGmCA*G*T*G*G*G*C*G*G*C*mCmGmUmGmA/iSp18//iSp18//3CholTEG/ (SEQ ID NO: 614), or a pharmaceutically acceptable salt thereof.

Embodiment 13. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmCmAmGmCA*G*A*G*G*G*A*C*A*A*mUmGmAmGmA/iSp18//iSp18//3CholTEG/ (SEQ ID NO: 829), or a pharmaceutically acceptable salt thereof.

Embodiment 14. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmCmCmAmGC*A*G*A*G*G*G*A*C*A*mAmUmGmAmG/iSp18//iSp18//3CholTEG/ (SEQ ID NO: 830), or a pharmaceutically acceptable salt thereof.

Embodiment 15. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmCmCmCmAG*C*A*G*A*G*G*G*A*C*mAmAmUmGmA/iSp18//iSp18//3CholTEG/ (SEQ ID NO: 831), or a pharmaceutically acceptable salt thereof.

Embodiment 16. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mAmGmCmCmAG*A*C*A*C*C*A*A*G*C*mAmCmUmUmU/iSp18//iSp18//3CholTEG/ (SEQ ID NO: 852), or a pharmaceutically acceptable salt thereof.

Embodiment 17. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmCmCmCmAG*C*C*A*G*A*C*A*C*C*mAmAmGmCmA/iSp18//iSp18//3CholTEG/ (SEQ ID NO: 856), or a pharmaceutically acceptable salt thereof.

Embodiment 18. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmUmGmGmGG*G*C*T*C*T*T*G*G*T*mUmGmUmCmC/iSp18//iSp18//3CholTEG/ (SEQ ID NO: 875), or a pharmaceutically acceptable salt thereof.

Embodiment 19. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmUmUmGmGG*G*G*C*T*C*T*T*G*G*mUmUmGmUmC/iSp18//iSp18//3CholTEG/ (SEQ ID NO: 876), or a pharmaceutically acceptable salt thereof.

Embodiment 20. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmAmCmUmUG*G*G*G*G*C*T*C*T*T*mGmGmUmUmG/iSp18//iSp18//3CholTEG/ (SEQ ID NO: 878), or a pharmaceutically acceptable salt thereof.

Embodiment 21. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of SEQ ID NO: 879, or a pharmaceutically acceptable salt thereof.

Embodiment 22. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmCmAmCmUT*G*G*G*G*G*C*T*C*T*mUmGmGmUmU/iSp18//iSp18//3CholTEG/ (SEQ ID NO: 891), or a pharmaceutically acceptable salt thereof.

Embodiment 23. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmAmCmUmGG*A*G*G*A*C*C*T*T*A*mGmGmGmAmA/iSp18//iSp18//3CholTEG/ (SEQ ID NO: 901), or a pharmaceutically acceptable salt thereof.

Embodiment 24. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmAmAmGmCA*C*T*G*G*A*G*G*A*C*mCmUmUmAmG/iSp18//iSp18//3CholTEG/ (SEQ ID NO: 905), or a pharmaceutically acceptable salt thereof.

Embodiment 25. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mAmUmUmCmAA*G*C*A*C*T*G*G*A*G*mGmAmCmCmU/iSp18//iSp18//3CholTEG/ (SEQ ID NO: 908), or a pharmaceutically acceptable salt thereof.

Embodiment 26. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmAmUmUmCA*A*G*C*A*C*T*G*G*A*mGmGmAmCmC/iSp18//iSp18//3CholTEG/ (SEQ ID NO: 909), or a pharmaceutically acceptable salt thereof.

Embodiment 27. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmGmAmUmAT*T*C*A*A*G*C*A*C*T*mGmGmAmGmG/iSp18//iSp18//3CholTEG/ (SEQ ID NO: 912), or a pharmaceutically acceptable salt thereof.

Embodiment 28. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmUmGmAmUA*T*T*C*A*A*G*C*A*C*mUmGmGmAmG/iSp18//iSp18//3CholTEG/ (SEQ ID NO: 913), or a pharmaceutically acceptable salt thereof.

Embodiment 29. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmUmCmUmCG*G*G*T*A*A*G*C*A*T*mCmCmUmCmG/iSp18//iSp18//3CholTEG/ (SEQ ID NO: 954), or a pharmaceutically acceptable salt thereof.

Embodiment 30. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmGmCmGmCA*G*A*A*C*A*T*G*G*T*mGmUmCmAmU/iSp18//iSp18//3CholTEG/ (SEQ ID NO: 980), or a pharmaceutically acceptable salt thereof.

Embodiment 31. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mAmCmAmGmGC*C*C*C*C*C*A*G*A*A*mUmCmAmCmC/iSp18//iSp18//3CholTEG/ (SEQ ID NO: 1030), or a pharmaceutically acceptable salt thereof.

Embodiment 32. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mAmGmGmGmUA*A*T*C*T*C*C*C*C*A*mGmGmAmCmA/iSp18//iSp18//3CholTEG/ (SEQ ID NO: 1079), or a pharmaceutically acceptable salt thereof.

Embodiment 33. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmAmCmUmUG*G*T*G*A*A*C*T*T*G*mCmAmGmAmG/iSp18//iSp18//3CholTEG/ (SEQ ID NO: 1135), or a pharmaceutically acceptable salt thereof.

Embodiment 34. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmUmUmUmCC*T*G*G*A*T*C*C*A*C*mUmUmGmGmU/iSp18//iSp18//3CholTEG/ (SEQ ID NO: 1147), or a pharmaceutically acceptable salt thereof.

Embodiment 35. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmGmUmUmUC*C*T*G*G*A*T*C*C*A*mCmUmUmGmG/iSp18//iSp18//3CholTEG/ (SEQ ID NO: 1148), or a pharmaceutically acceptable salt thereof.

Embodiment 36. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmUmGmGmAT*G*G*T*T*T*C*C*T*G*mGmAmUmCmC/iSp18//iSp18//3CholTEG/ (SEQ ID NO: 1154), or a pharmaceutically acceptable salt thereof.

Embodiment 37. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmCmUmGmGA*T*G*G*T*T*T*C*C*T*mGmGmAmUmC/iSp18//iSp18//3CholTEG/ (SEQ ID NO: 1155), or a pharmaceutically acceptable salt thereof.

Embodiment 38. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmCmCmUmGG*A*T*G*G*T*T*T*C*C*mUmGmGmAmU/iSp18//iSp18//3CholTEG/ (SEQ ID NO: 1156), or a pharmaceutically acceptable salt thereof.

Embodiment 39. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmGmCmCmUG*G*A*T*G*G*T*T*T*C*mCmUmGmGmA/iSp18//iSp18//3CholTEG/ (SEQ ID NO: 1157), or a pharmaceutically acceptable salt thereof.

Embodiment 40. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mAmGmUmUmGG*C*C*T*G*G*A*T*G*G*mUmUmUmCmC/iSp18//iSp18//3CholTEG/ (SEQ ID NO: 1161), or a pharmaceutically acceptable salt thereof.

Embodiment 41. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmAmGmUmUG*G*C*C*T*G*G*A*T*G*mGmUmUmUmC/iSp18//iSp18//3CholTEG/ (SEQ ID NO: 1162), or a pharmaceutically acceptable salt thereof.

Embodiment 42. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmGmAmGmUT*G*G*C*C*T*G*G*A*T*mGmGmUmUmU/iSp18//iSp18//3CholTEG/ (SEQ ID NO: 1163), or a pharmaceutically acceptable salt thereof.

Embodiment 43. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmAmGmGmAG*T*T*G*G*C*C*T*G*G*mAmUmGmGmU/iSp18//iSp18//3CholTEG/ (SEQ ID NO: 1165), or a pharmaceutically acceptable salt thereof.

Embodiment 44. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmCmAmGmGA*G*T*T*G*G*C*C*T*G*mGmAmUmGmG/iSp18//iSp18//3CholTEG/ (SEQ ID NO: 1166), or a pharmaceutically acceptable salt thereof.

Embodiment 45. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmGmGmAmUG*A*C*T*C*A*G*G*A*G*mUmUmGmGmC/iSp18//iSp18//3CholTEG/ (SEQ ID NO: 1174), or a pharmaceutically acceptable salt thereof.

Embodiment 46. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmGmUmGmUG*C*T*G*A*G*T*C*C*T*mGmGmGmAmU/iSp18//iSp18//3CholTEG/ (SEQ ID NO: 1189), or a pharmaceutically acceptable salt thereof.

Embodiment 47. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmGmAmAmGG*A*A*T*G*A*G*G*G*T*mCmUmGmAmA/iSp18//iSp18//3CholTEG/ (SEQ ID NO: 1248), or a pharmaceutically acceptable salt thereof.

Embodiment 48. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmAmAmCmAT*C*T*C*T*G*G*G*A*A*mGmGmAmAmU/iSp18//iSp18//3CholTEG/ (SEQ ID NO: 1259), or a pharmaceutically acceptable salt thereof.

Embodiment 49. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmCmUmCmAA*C*A*T*C*T*C*T*G*G*mGmAmAmGmG/iSp18//iSp18//3CholTEG/ (SEQ ID NO: 1262), or a pharmaceutically acceptable salt thereof.

Embodiment 50. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmAmUmUmCT*C*A*A*C*A*T*C*T*C*mUmGmGmGmA/iSp18//iSp18//3CholTEG/ (SEQ ID NO: 1265), or a pharmaceutically acceptable salt thereof.

Embodiment 51. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mAmCmAmUmUC*T*C*A*A*C*A*T*C*T*mCmUmGmGmG/iSp18//iSp18//3CholTEG/ (SEQ ID NO: 1266), or a pharmaceutically acceptable salt thereof.

Embodiment 52. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mAmAmCmAmUT*C*T*C*A*A*C*A*T*C*mUmCmUmGmG/iSp18//iSp18//3CholTEG/ (SEQ ID NO: 1267), or a pharmaceutically acceptable salt thereof.

Embodiment 53. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmGmAmAmCA*T*T*C*T*C*A*A*C*A*mUmCmUmCmU/iSp18//iSp18//3CholTEG/ (SEQ ID NO: 1269), or a pharmaceutically acceptable salt thereof.

Embodiment 54. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmAmGmAmGA*T*G*A*A*C*A**T**C*mUmCmAmAmC/iSp18//iSp18//3CholTEG/ (SEQ ID NO: 1275), or a pharmaceutically acceptable salt thereof.

Embodiment 55. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmGmGmAmGA*G*A*T*G*A*A*C*A*T*mUmCmUmCmA/iSp18//iSp18//3CholTEG/ (SEQ ID NO: 1277), or a pharmaceutically acceptable salt thereof.

Embodiment 56. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmUmGmGmAG*A*G*A*T*G*A*A*C*A*mUmUmCmUmC/iSp18//iSp18//3CholTEG/ (SEQ ID NO: 1278), or a pharmaceutically acceptable salt thereof.

Embodiment 57. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmCmUmGmGA*G*A*G*A*T*G*A*A*C*mAmUmUmCmU/iSp18//iSp18//3CholTEG/ (SEQ ID NO: 1279), or a pharmaceutically acceptable salt thereof.

Embodiment 58. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmAmUmGmGG*G*T*C*A*G*G*G*G*C*mUmGmGmAmG/iSp18//iSp18//3CholTEG/ (SEQ ID NO: 1292), or a pharmaceutically acceptable salt thereof.

Embodiment 59. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmCmAmGmAC*C*C*T*G*A*G*T*C*C*mAmGmGmAmG/iSp18//iSp18//3CholTEG/ (SEQ ID NO: 1312), or a pharmaceutically acceptable salt thereof.

Embodiment 60. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmGmUmCmAG*C*C*C*A*A*T*G*T*G*mGmGmGmGmA/iSp18//iSp18//3CholTEG/ (SEQ ID NO: 1334), or a pharmaceutically acceptable salt thereof.

Embodiment 61. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmGmGmUmCA*G*C*C*C*A*A*T*G*T*mGmGmGmGmG/iSp18//iSp18//3CholTEG/ (SEQ ID NO: 1335), or a pharmaceutically acceptable salt thereof.

Embodiment 62. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmAmCmGmGT*C*A*G*C*C*C*A*A*T*mGmUmGmGmG/iSp18//iSp18//3CholTEG/ (SEQ ID NO: 1337), or a pharmaceutically acceptable salt thereof.

Embodiment 63. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmAmGmAmCA*C*G*G*T*C*A*G*C*C*mCmAmAmUmG/iSp18//iSp18//3CholTEG/ (SEQ ID NO: 1341), or a pharmaceutically acceptable salt thereof.

Embodiment 64. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mAmGmAmGmAC*A*C*G*G*T*C*A*G*C*mCmCmAmAmU/iSp18//iSp18//3CholTEG/ (SEQ ID NO: 1342), or a pharmaceutically acceptable salt thereof.

Embodiment 65. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmAmGmAmGA*C*A*C*G*G*T*C*A*G*mCmCmCmAmA/iSp18//iSp18//3CholTEG/ (SEQ ID NO: 1343), or a pharmaceutically acceptable salt thereof.

Embodiment 66. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mAmGmAmGmAG*A*C*A*C*G*G*T*C*A*mGmCmCmCmA/iSp18//iSp18//3CholTEG/ (SEQ ID NO: 1344), or a pharmaceutically acceptable salt thereof.

Embodiment 67. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmAmGmAmGA*G*A*C*A*C*G*G*T*C*mAmGmCmCmC/iSp18//iSp18//3CholTEG/ (SEQ ID NO: 1345), or a pharmaceutically acceptable salt thereof.

Embodiment 68. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmUmAmGmAG*A*G*A*C*A*C*G*G*T*mCmAmGmCmC/iSp18//iSp18//3CholTEG/ (SEQ ID NO: 1346), or a pharmaceutically acceptable salt thereof.

Embodiment 69. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmGmAmAmAT*T*G*T*T*C*C*C*A*G*mGmGmUmUmC/iSp18//iSp18//3CholTEG/ (SEQ ID NO: 1368), or a pharmaceutically acceptable salt thereof.

Embodiment 70. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mGmAmGmAmCG*C*A*A*C*C*C*C*C*G*mCmCmCmUmG/iSp18//iSp18//3ChoTEG/(SEQ ID NO: 1397), or a pharmaceutically acceptable salt thereof.

Embodiment 71. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mAmAmAmUmUT*G*G*G*T*C*A*G*A*G*mCmUmGmCmA/iSp18//iSp18//3CholTEG/ (SEQ ID NO: 1466), or a pharmaceutically acceptable salt thereof.

Embodiment 72. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmUmGmGmGA*C*T*A*A*A*T*T*T*G*mGmGmUmCmA/iSp18//iSp18//3CholTEG/ (SEQ ID NO: 1474), or a pharmaceutically acceptable salt thereof.

Embodiment 73. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mUmCmUmGmGG*A*C*T*A*A*A*T*T*T*mGmGmGmUmC/iSp18//iSp18//3CholTEG/ (SEQ ID NO: 1475), or a pharmaceutically acceptable salt thereof.

Embodiment 74. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of mCmAmCmGmGmUmGmUmUmAmGmAmGmGmGmGmUmGmGmU/iSp18//iSp18//3CholTEG/ (SEQ ID NO: 1624), or a pharmaceutically acceptable salt thereof.

Embodiment 75. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 1997, or a pharmaceutically acceptable salt thereof.

Embodiment 76. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2007, or a pharmaceutically acceptable salt thereof.

Embodiment 77. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2010, or a pharmaceutically acceptable salt thereof.

Embodiment 78. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2011, or a pharmaceutically acceptable salt thereof.

Embodiment 79. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2012, or a pharmaceutically acceptable salt thereof.

Embodiment 80. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2173, or a pharmaceutically acceptable salt thereof.

Embodiment 81. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2241, or a pharmaceutically acceptable salt thereof.

Embodiment 82. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2269, or a pharmaceutically acceptable salt thereof.

Embodiment 83. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 465, or a pharmaceutically acceptable salt thereof.

Embodiment 84. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2343, or a pharmaceutically acceptable salt thereof.

Embodiment 85. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2412, or a pharmaceutically acceptable salt thereof.

Embodiment 86. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2429, or a pharmaceutically acceptable salt thereof.

Embodiment 87. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2624, or a pharmaceutically acceptable salt thereof.

Embodiment 88. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2625, or a pharmaceutically acceptable salt thereof.

Embodiment 89. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2626, or a pharmaceutically acceptable salt thereof.

Embodiment 90. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2641, or a pharmaceutically acceptable salt thereof.

Embodiment 91. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 856, or a pharmaceutically acceptable salt thereof.

Embodiment 92. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2660, or a pharmaceutically acceptable salt thereof.

Embodiment 93. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2661, or a pharmaceutically acceptable salt thereof.

Embodiment 94. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2663, or a pharmaceutically acceptable salt thereof.

Embodiment 95. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2664, or a pharmaceutically acceptable salt thereof.

Embodiment 96. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2675, or a pharmaceutically acceptable salt thereof.

Embodiment 97. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2685, or a pharmaceutically acceptable salt thereof.

Embodiment 98. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2689, or a pharmaceutically acceptable salt thereof.

Embodiment 99. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2692, or a pharmaceutically acceptable salt thereof.

Embodiment 100. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2693, or a pharmaceutically acceptable salt thereof.

Embodiment 101. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2696, or a pharmaceutically acceptable salt thereof.

Embodiment 102. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2697, or a pharmaceutically acceptable salt thereof.

Embodiment 103. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2736, or a pharmaceutically acceptable salt thereof.

Embodiment 104. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2762, or a pharmaceutically acceptable salt thereof.

Embodiment 105. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2807, or a pharmaceutically acceptable salt thereof.

Embodiment 106. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2840, or a pharmaceutically acceptable salt thereof.

Embodiment 107. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2883, or a pharmaceutically acceptable salt thereof.

Embodiment 108. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2895, or a pharmaceutically acceptable salt thereof.

Embodiment 109. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2896, or a pharmaceutically acceptable salt thereof.

Embodiment 110. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2902, or a pharmaceutically acceptable salt thereof.

Embodiment 111. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2903, or a pharmaceutically acceptable salt thereof.

Embodiment 112. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2904, or a pharmaceutically acceptable salt thereof.

Embodiment 113. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2905, or a pharmaceutically acceptable salt thereof.

Embodiment 114. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2909, or a pharmaceutically acceptable salt thereof.

Embodiment 115. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2910, or a pharmaceutically acceptable salt thereof.

Embodiment 116. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2911, or a pharmaceutically acceptable salt thereof.

Embodiment 117. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2913, or a pharmaceutically acceptable salt thereof.

Embodiment 118. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2914, or a pharmaceutically acceptable salt thereof.

Embodiment 119. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2922, or a pharmaceutically acceptable salt thereof.

Embodiment 120. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2937, or a pharmaceutically acceptable salt thereof.

Embodiment 121. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2991, or a pharmaceutically acceptable salt thereof.

Embodiment 122. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3002, or a pharmaceutically acceptable salt thereof.

Embodiment 123. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3005, or a pharmaceutically acceptable salt thereof.

Embodiment 124. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3008, or a pharmaceutically acceptable salt thereof.

Embodiment 125. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3009, or a pharmaceutically acceptable salt thereof.

Embodiment 126. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3010, or a pharmaceutically acceptable salt thereof.

Embodiment 127. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3012, or a pharmaceutically acceptable salt thereof.

Embodiment 128. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3017, or a pharmaceutically acceptable salt thereof.

Embodiment 129. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3019, or a pharmaceutically acceptable salt thereof.

Embodiment 130. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3020, or a pharmaceutically acceptable salt thereof.

Embodiment 131. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3021, or a pharmaceutically acceptable salt thereof.

Embodiment 132. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3033, or a pharmaceutically acceptable salt thereof.

Embodiment 133. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 1312, or a pharmaceutically acceptable salt thereof.

Embodiment 134. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3062, or a pharmaceutically acceptable salt thereof.

Embodiment 135. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3063, or a pharmaceutically acceptable salt thereof.

Embodiment 136. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3065, or a pharmaceutically acceptable salt thereof.

Embodiment 137. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3069, or a pharmaceutically acceptable salt thereof.

Embodiment 138. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3070, or a pharmaceutically acceptable salt thereof.

Embodiment 139. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 1343, or a pharmaceutically acceptable salt thereof.

Embodiment 140. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 1344, or a pharmaceutically acceptable salt thereof.

Embodiment 141. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3071, or a pharmaceutically acceptable salt thereof.

Embodiment 142. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3072, or a pharmaceutically acceptable salt thereof.

Embodiment 143. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3091, or a pharmaceutically acceptable salt thereof.

Embodiment 144. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3118, or a pharmaceutically acceptable salt thereof.

Embodiment 145. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3178, or a pharmaceutically acceptable salt thereof.

Embodiment 146. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3186, or a pharmaceutically acceptable salt thereof.

Embodiment 147. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3187, or a pharmaceutically acceptable salt thereof.

Embodiment 148. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 2248, or a pharmaceutically acceptable salt thereof.

Embodiment 149. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mCmGmAmGA*G*G*A*T*C*T*G*A*T*mGmUmG/iSp18//iSp18//3CholTEG/ (SEQ ID NO: 3449), or a pharmaceutically acceptable salt thereof.

Embodiment 150. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mUmGmGmAG*C*T*G*A*T*T*G*C*C*mAmCmU/iSp18//iSp18//3CholTEG/ (SEQ ID NO: 3670), or a pharmaceutically acceptable salt thereof.

Embodiment 151. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mCmCmGmCA*G*T*G*G*A*G*C*T*G*mAmUmU/iSp18//iSp18//3CholTEG/ (SEQ ID NO: 3676), or a pharmaceutically acceptable salt thereof.

Embodiment 152. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mCmAmCmCC*A*G*C*G*C*T*C*A*T*mUmGmA/iSp18//iSp18//3CholTEG/ (SEQ ID NO: 3704), or a pharmaceutically acceptable salt thereof.

Embodiment 153. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mGmAmGmCA*C*C*C*A*G*C*G*C*T*mCmAmU/iSp18//iSp18//3CholTEG/ (SEQ ID NO: 3707), or a pharmaceutically acceptable salt thereof.

Embodiment 154. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mCmAmGmGT*G*C*A*C*G*G*T*G*T*mAmCmU/iSp18//iSp18//3CholTEG/ (SEQ ID NO: 3749), or a pharmaceutically acceptable salt thereof.

Embodiment 155. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mGmCmCmCA*G*G*T*G*C*A*C*G*G*mUmGmU/iSp18//iSp18//3CholTEG/ (SEQ ID NO: 3752), or a pharmaceutically acceptable salt thereof.

Embodiment 156. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mGmAmGmGT*C*A*G*A*G*G*G*A*A*mAmGmG/iSp18//iSp18//3CholTEG/ (SEQ ID NO: 3998), or a pharmaceutically acceptable salt thereof.

Embodiment 157. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mUmCmCmCC*C*T*G*A*G*T*C*A*C*mCmAmU/iSp18//iSp18//3CholTEG/ (SEQ ID NO: 4130), or a pharmaceutically acceptable salt thereof.

Embodiment 158. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mGmGmAmGG*C*T*G*A*A*G*C*A*C*mGmAmG/iSp18//iSp18//3CholTEG/ (SEQ ID NO: 4868), or a pharmaceutically acceptable salt thereof.

Embodiment 159. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mUmUmGmGG*A*G*G*C*C*A*A*G*G*mUmGmG/iSp18//iSp18//3CholTEG/ (SEQ ID NO: 5009), or a pharmaceutically acceptable salt thereof.

Embodiment 160. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mUmGmUmAA*T*C*C*C*A*G*C*A*C*mUmUmU/iSp18//iSp18//3CholTEG/ (SEQ ID NO: 5024), or a pharmaceutically acceptable salt thereof.

Embodiment 161. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mCmCmUmGT*A*A*T*C*C*C*A*G*C*mAmCmU/iSp18//iSp18//3CholTEG/ (SEQ ID NO: 5026), or a pharmaceutically acceptable salt thereof.

Embodiment 162. A synthetic oligonucleotide comprising a nucleic acid sequence complementary to or sufficiently complementary to a region of a kallikrein-related peptidase 7 (KLK7) gene and/or to a region of a KLK7gene product, wherein the synthetic oligonucleotide comprises, consists essentially of, or consists of mGmCmGmAmGmGmAmCmCmAmGmAmAmGmGmGmC/iSp18//iSp18//3CholTEG/ (SEQ ID NO: 5376), or a pharmaceutically acceptable salt thereof.

Embodiment 163. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mAmGmGmGmAmGmAmGmCmUmGmUmCmAmGmUmC/iSp18//iSp18//3CholTEG/ (SEQ ID NO: 5406), or a pharmaceutically acceptable salt thereof.

Embodiment 164. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mGmGmAmGmCmAmGmGmGmAmGmAmGmCmUmGmU/iSp18//iSp18//3CholTEG/ (SEQ ID NO: 5411), or a pharmaceutically acceptable salt thereof.

Embodiment 165. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mAmGmGmAmGmCmAmGmGmGmAmGmAmGmCmUmG/iSp18//iSp18//3CholTEG/ (SEQ ID NO: 5412), or a pharmaceutically acceptable salt thereof.

Embodiment 166. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mGmGmUmCmAmCmUmGmCmAmGmGmGmAmGmGmA/iSp18//iSp18//3CholTEG/ (SEQ ID NO: 5425), or a pharmaceutically acceptable salt thereof.

Embodiment 167. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mAmAmAmGmGmUmCmAmCmUmGmCmAmGmGmGmA/iSp18//iSp18//3CholTEG/ (SEQ ID NO: 5428), or a pharmaceutically acceptable salt thereof.

Embodiment 168. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mGmGmGmAmAmAmGmGmUmCmAmCmUmGmCmAmG/iSp18//iSp18//3CholTEG/ (SEQ ID NO: 5431), or a pharmaceutically acceptable salt thereof.

Embodiment 169. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mAmGmAmGmGmGmAmAmAmGmGmUmCmAmCmUmG/iSp18//iSp18//3CholTEG/ (SEQ ID NO: 5434), or a pharmaceutically acceptable salt thereof.

Embodiment 170. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mGmGmUmCmAmGmAmGmGmGmAmAmAmGmGmUmC/iSp18//iSp18//3CholTEG/ (SEQ ID NO: 3996), or a pharmaceutically acceptable salt thereof.

Embodiment 171. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mAmGmGmUmCmAmGmAmGmGmGmAmAmAmGmGmU/iSp18//iSp18//3CholTEG/ (SEQ ID NO: 3997), or a pharmaceutically acceptable salt thereof.

Embodiment 172. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mGmAmGmGmUmCmAmGmAmGmGmGmAmAmAmGmG/iSp18//iSp18//3CholTEG/ (SEQ ID NO: 5437), or a pharmaceutically acceptable salt thereof.

Embodiment 173. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mUmGmAmGmGmUmCmAmGmAmGmGmGmAmAmAmG/iSp18//iSp18//3CholTEG/ (SEQ ID NO: 5438), or a pharmaceutically acceptable salt thereof.

Embodiment 174. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mGmCmAmCmAmUmGmAmGmGmUmCmAmGmAmGmG/iSp18//iSp18//3CholTEG/ (SEQ ID NO: 5443), or a pharmaceutically acceptable salt thereof.

Embodiment 175. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mCmGmCmAmCmAmUmGmAmGmGmUmCmAmGmAmG/iSp18//iSp18//3CholTEG/ (SEQ ID NO: 5444), or a pharmaceutically acceptable salt thereof.

Embodiment 176. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mAmCmGmCmAmCmAmUmGmAmGmGmUmCmAmGmA/iSp18//iSp18//3CholTEG/ (SEQ ID NO: 5445), or a pharmaceutically acceptable salt thereof.

Embodiment 177. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mCmAmCmGmCmAmCmAmUmGmAmGmGmUmCmAmG/iSp18//iSp18//3CholTEG/ (SEQ ID NO: 5446), or a pharmaceutically acceptable salt thereof.

Embodiment 178. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mCmCmAmCmGmCmAmCmAmUmGmAmGmGmUmCmA/iSp18//iSp18//3CholTEG/ (SEQ ID NO: 5447), or a pharmaceutically acceptable salt thereof.

Embodiment 179. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mUmCmCmAmCmGmCmAmCmAmUmGmAmGmGmUmC/iSp18//iSp18//3CholTEG/ (SEQ ID NO: 5448), or a pharmaceutically acceptable salt thereof.

Embodiment 180. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mUmUmGmAmCmAmUmCmCmAmCmGmCmAmCmAmU/iSp18//iSp18//3CholTEG/ (SEQ ID NO: 5454), or a pharmaceutically acceptable salt thereof.

Embodiment 181. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mGmCmGmUmUmUmUmUmCmUmUmGmGmAmGmUmC/iSp18//iSp18//3CholTEG/ (SEQ ID NO: 5542), or a pharmaceutically acceptable salt thereof.

Embodiment 182. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of mUmAmCmCmCmAmGmGmAmCmUmGmGmGmGmAmG/iSp18//iSp18//3CholTEG/ (SEQ ID NO: 5588), or a pharmaceutically acceptable salt thereof.

Embodiment 183. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 5687, or a pharmaceutically acceptable salt thereof.

Embodiment 184. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 5879, or a pharmaceutically acceptable salt thereof.

Embodiment 185. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 5885, or a pharmaceutically acceptable salt thereof.

Embodiment 186. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 5901, or a pharmaceutically acceptable salt thereof.

Embodiment 187. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 5904, or a pharmaceutically acceptable salt thereof.

Embodiment 188. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 5940, or a pharmaceutically acceptable salt thereof.

Embodiment 189. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 5943, or a pharmaceutically acceptable salt thereof.

Embodiment 190. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 3998, or a pharmaceutically acceptable salt thereof. Embodiment 191. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 6267, or a pharmaceutically acceptable salt thereof.

Embodiment 192. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 4868, or a pharmaceutically acceptable salt thereof.

Embodiment 193. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 7060, or a pharmaceutically acceptable salt thereof.

Embodiment 194. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 7070, or a pharmaceutically acceptable salt thereof.

Embodiment 195. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 7072, or a pharmaceutically acceptable salt thereof.

Embodiment 196. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 5376, or a pharmaceutically acceptable salt thereof.

Embodiment 197. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 7417, or a pharmaceutically acceptable salt thereof.

Embodiment 198. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 7422, or a pharmaceutically acceptable salt thereof.

Embodiment 199. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 7423, or a pharmaceutically acceptable salt thereof.

Embodiment 200. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 7431, or a pharmaceutically acceptable salt thereof.

Embodiment 201. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 7434, or a pharmaceutically acceptable salt thereof.

Embodiment 202. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 7437, or a pharmaceutically acceptable salt thereof.

Embodiment 203. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 7440, or a pharmaceutically acceptable salt thereof.

Embodiment 204. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 6153, or a pharmaceutically acceptable salt thereof.

Embodiment 205. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 6154, or a pharmaceutically acceptable salt thereof.

Embodiment 206. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 3998, or a pharmaceutically acceptable salt thereof.

Embodiment 207. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 6155, or a pharmaceutically acceptable salt thereof.

Embodiment 208. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 4004, or a pharmaceutically acceptable salt thereof.

Embodiment 209. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 4005, or a pharmaceutically acceptable salt thereof.

Embodiment 210. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 4006, or a pharmaceutically acceptable salt thereof.

Embodiment 211. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 4007, or a pharmaceutically acceptable salt thereof.

Embodiment 212. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 6159, or a pharmaceutically acceptable salt thereof.

Embodiment 213. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 6160, or a pharmaceutically acceptable salt thereof.

Embodiment 214. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 6166, or a pharmaceutically acceptable salt thereof.

Embodiment 215. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 7516, or a pharmaceutically acceptable salt thereof.

Embodiment 216. A spherical nucleic acid (SNA) comprising a core and an oligonucleotide shell, wherein the oligonucleotide comprises a first synthetic oligonucleotide complementary to or sufficiently complementary to a region of a first kallikrein-related peptidase (KK) gene and/or to a region of a first KLK gene product, and a second synthetic oligonucleotide complementary to or sufficiently complementary to a region of a second KLK gene and/or to a region of a second KLK gene product, wherein the first region and the second region of the KLK gene and/or the KLK gene product are within the same intron, exon, 3′-untranslated region (UTR), or 5′-UTR.

Embodiment 217. A spherical nucleic acid (SNA) comprising a core and an oligonucleotide shell, wherein the oligonucleotide comprises a first synthetic oligonucleotide complementary to or sufficiently complementary to a region of a first kallikrein-related peptidase (KLK) gene and/or to a region of a first KLK gene product, and a second synthetic oligonucleotide complementary to or sufficiently complementary to a region of a second KLK gene and/or to a region of a second KLK gene product, wherein the first region and the second region in the KLK gene and/or the KLK gene product are not within the same intron, exon, 3′-untranslated region (UTR), or 5′-UTR.

Embodiment 218. A spherical nucleic acid (SNA) comprising a core and an oligonucleotide shell, wherein the oligonucleotide comprises a first synthetic oligonucleotide complementary to or sufficiently complementary to a region of a first kallikrein-related peptidase (KLK) gene and/or to a region of a first KLK gene product, and a second synthetic oligonucleotide complementary to or sufficiently complementary to a region of a second KLK gene and/or to a region of a second KLK gene product, wherein the first region is within an exon.

Embodiment 219. A spherical nucleic acid (SNA) comprising a core and an oligonucleotide shell, wherein the oligonucleotide comprises a first synthetic oligonucleotide complementary to or sufficiently complementary to a region of a first kallikrein-related peptidase (KLK) gene and/or to a region of a first KLK gene product, and a second synthetic oligonucleotide complementary to or sufficiently complementary to a region of a second KLK gene and/or to a region of a second KLK gene product, wherein the second region is within an exon.

Embodiment 220. A spherical nucleic acid (SNA) comprising a core and an oligonucleotide shell, wherein the oligonucleotide comprises a first synthetic oligonucleotide complementary to or sufficiently complementary to a region of a first kallikrein-related peptidase (KLK) gene and/or to a region of a first KLK gene product, and a second synthetic oligonucleotide complementary to or sufficiently complementary to a region of a second KLK gene and/or to a region of a second KLK gene product, wherein the first region is within a 3′- or 5′-untranslated region (3′-UTR or 5′-UTR).

Embodiment 221. A spherical nucleic acid (SNA) comprising a core and an oligonucleotide shell, wherein the oligonucleotide comprises a first synthetic oligonucleotide complementary to or sufficiently complementary to a region of a first kallikrein-related peptidase (KLK) gene and/or to a region of a first KLK gene product, and a second synthetic oligonucleotide complementary to or sufficiently complementary to a region of a second KLK gene and/or to a region of a second KLK gene product, wherein the second region is within a 3′-UTR or 5′-UTR.

Embodiment 222. A spherical nucleic acid (SNA) comprising a core and an oligonucleotide shell, wherein the oligonucleotide comprises a first synthetic oligonucleotide complementary to or sufficiently complementary to a region of a first kallikrein-related peptidase (KLK) gene and/or to a region of a first KLK gene product, and a second synthetic oligonucleotide complementary to or sufficiently complementary to a region of a second KLK gene and/or to a region of a second KLK gene product, wherein the first region is within an intron.

Embodiment 223. A spherical nucleic acid (SNA) comprising a core and an oligonucleotide shell, wherein the oligonucleotide comprises a first synthetic oligonucleotide complementary to or sufficiently complementary to a region of a first kallikrein-related peptidase (KLK) gene and/or to a region of a first KLK gene product, and a second synthetic oligonucleotide complementary to or sufficiently complementary to a region of a second KLK gene and/or to a region of a second KLK gene product, wherein the second region is within an intron.

Embodiment 224. A pharmaceutical composition comprising:
a first spherical nucleic acid (SNA) that comprises a core and an oligonucleotide shell, wherein the oligonucleotide shell comprises a first synthetic oligonucleotide comprising a first nucleic acid sequence complementary to or sufficiently complementary to a region of a first kallikrein-related peptidase (KLK) gene and/or to a region of a first KLK gene product, and a second SNA that comprises a core and an oligonucleotide shell, wherein the oligonucleotide shell comprises a second synthetic oligonucleotide comprising a second nucleic acid sequence complementary to or sufficiently complementary to a region of a second KLK gene and/or to a region of a second KLK gene product.

Embodiment 225. The pharmaceutical composition of Embodiment 224, wherein the first KLK gene is a KLK5 gene.

Embodiment 226. The pharmaceutical composition of Embodiment 224, wherein the first KLK gene product is a KLK5 gene product.

Embodiment 227. The pharmaceutical composition of Embodiment 224, wherein the second KLK gene is a KLK7 gene.

Embodiment 228. The pharmaceutical composition of Embodiment 224, wherein the second KLK gene product is a KLK7 gene product.

Embodiment 229. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of any one of SEQ ID NOs: 1-3302, and/or the synthetic oligonucleotide comprises, consists essentially of, or consists of a synthetic oligonucleotide provided in Table 1, or a pharmaceutically acceptable salt thereof.

Embodiment 230. A synthetic oligonucleotide comprising a nucleic acid sequence complementary to or sufficiently complementary to a region of a kallikrein-related peptidase 7 (KLK7) gene and/or to a region of a KLK7 gene product, or a pharmaceutically acceptable salt thereof.

Embodiment 231. The synthetic oligonucleotide of embodiment 230, wherein the nucleic acid sequence comprises, consists essentially of, or consists of any one of SEQ ID NOs: 3401-7574, and/or the synthetic oligonucleotide comprises, consists essentially of, or consists of a synthetic oligonucleotide provided in Table 2, or a pharmaceutically acceptable salt thereof.

Embodiment 232. The synthetic oligonucleotide of embodiment 229, wherein the nucleic acid sequence comprises, consists essentially of, or consists of any one of SEQ ID NO: 99, SEQ ID NO: 109, SEQ ID NO: 112, SEQ ID NO: 113, SEQ ID NO: 114, SEQ ID NO: 315, SEQ ID NO: 403, SEQ ID NO: 434, SEQ ID NO: 465, SEQ ID NO: 516, SEQ ID NO: 596, SEQ ID NO: 614, SEQ ID NO: 829, SEQ ID NO: 830, SEQ ID NO: 831, SEQ iD NO: 852, SEQ ID NO: 856, SEQ ID NO: 875, SEQ ID NO: 876, SEQ ID NO: 878, SEQ ID NO: 879, SEQ ID NO: 891, SEQ ID NO: 901, SEQ ID NO: 905, SEQ ID NO: 908, SEQ ID NO: 909, SEQ ID NO: 912, SEQ ID NO: 913, SEQ ID NO: 954, SEQ ID NO: 980, SEQ ID NO: 1030, SEQ ID NO: 1079, SEQ ID NO: 1135, SEQ ID NO: 1147, SEQ ID NO: 1148, SEQ ID NO: 1154, SEQ ID NO: 1155, SEQ ID NO: 1156, SEQ ID NO: 1157, SEQ ID NO: 1161, SEQ ID NO: 1162, SEQ ID NO: 1163, SEQ ID NO: 1165, SEQ ID NO: 1166, SEQ ID NO: 1174, SEQ ID NO: 1189, SEQ ID NO: 1248, SEQ ID NO: 1259, SEQ ID NO: 1262, SEQ ID NO: 1265, SEQ ID NO: 1266, SEQ ID NO: 1267, SEQ ID NO: 1269, SEQ ID NO: 1275, SEQ ID NO: 1277, SEQ ID NO: 1278, SEQ ID NO: 1279, SEQ ID NO: 1292, SEQ ID NO: 1312, SEQ ID NO: 1334, SEQ ID NO: 1335, SEQ ID NO: 1337, SEQ ID NO: 1341, SEQ ID NO: 1342, SEQ ID NO: 1343, SEQ ID NO: 1344, SEQ ID NO: 1345, SEQ ID NO: 1346, SEQ ID NO: 1368, SEQ ID NO: 1397, SEQ ID NO: 1466, SEQ ID NO: 1474, SEQ ID NO: 1475, SEQ ID NO: 1624, SEQ ID NO: 1997, SEQ ID NO: 2007, SEQ ID NO: 2010, SEQ ID NO: 2011, SEQ ID NO: 2012, SEQ ID NO: 2173, SEQ ID NO: 2241, SEQ ID NO: 2269, SEQ ID NO: 465, SEQ ID NO: 2343, SEQ ID NO: 2412, SEQ ID NO: 2429, SEQ ID NO: 2624, SEQ ID NO: 2625, SEQ ID NO: 2626, SEQ ID NO: 2641, SEQ ID NO: 856, SEQ ID NO: 2660, SEQ ID NO: 2661, SEQ ID NO: 2663, SEQ ID NO: 2664, SEQ ID NO: 2675, SEQ ID NO: 2685, SEQ ID NO: 2689, SEQ ID NO: 2692, SEQ ID NO: 2693, SEQ ID NO: 2696, SEQ ID NO: 2697, SEQ ID NO: 2736, SEQ ID NO: 2762, SEQ ID NO: 2807, SEQ ID NO: 2840, SEQ ID NO: 2883, SEQ ID NO: 2895, SEQ ID NO: 2896, SEQ ID NO: 2902, SEQ ID NO: 2903, SEQ ID NO: 2904, SEQ ID NO: 2905, SEQ ID NO: 2909, SEQ ID NO: 2910, SEQ ID NO: 2911, SEQ ID NO: 2913, SEQ ID NO: 2914, SEQ ID NO: 2922, SEQ ID NO: 2937, SEQ ID NO: 2991, SEQ ID NO: 3002, SEQ ID NO: 3005, SEQ ID NO: 3008, SEQ ID NO: 3009, SEQ ID NO: 3010, SEQ ID NO: 3012, SEQ ID NO: 3017, SEQ ID NO: 3019, SEQ ID NO: 3020, SEQ ID NO: 3021, SEQ ID NO: 3033, SEQ ID NO: 1312, SEQ ID NO: 3062, SEQ ID NO: 3063, SEQ ID NO: 3065, SEQ ID NO: 3069, SEQ ID NO: 3070, SEQ ID NO: 1343, SEQ ID NO: 1344, SEQ ID NO: 3071, SEQ ID NO: 3072, SEQ ID NO: 3091, SEQ ID NO: 3118, SEQ ID NO: 3178, SEQ ID NO: 3186, SEQ ID NO: 3187, or SEQ ID NO: 2248, and/or wherein the synthetic oligonucleotide comprises, consists essentially of, or consists of any one of

(SEQ ID NO: 99)
mCmUmGmAmGC*C*A*C*C*C*T*C*A*C*mCmAmGmGmU/iSp18//iSp18//3CholTEG/,
(SEQ ID NO: 109)
mUmCmCmCmUG*C*C*T*G*C*T*G*A*G*mCmCmAmCmC/iSp18//iSp18//3CholTEG/,
(SEQ ID NO: 112)
mCmCmUmUmCC*C*T*G*C*C*T*G*C*T*mGmAmGmCmC/iSp18//iSp18//3CholTEG/,
(SEQ ID NO: 113)
mUmCmCmUmUC*C*C*T*G*C*C*T*G*C*mUmGmAmGmC/iSp18//iSp18//3CholTEG/,
(SEQ ID NO: 114)
mCmUmCmCmUT*C*C*C*T*G*C*C*T*G*mCmUmGmAmG/iSp18//iSp18//3CholTEG/,
(SEQ ID NO: 315)
mCmCmCmAmCA*T*C*C*A*G*G*G*G*G*mGmUmCmUmU/iSp18//iSp18//3CholTEG/,
(SEQ ID NO: 403)
mUmUmAmGmAG*G*G*G*T*G*G*T*C*A*mCmAmGmGmA/iSp18//iSp18//3CholTEG/,
(SEQ ID NO: 434)
mCmAmGmGmUC*C*T*G*G*T*T*G*C*T*mCmCmCmAmG/iSp18//iSp18//3CholTEG/,
(SEQ ID NO: 465)
mCmCmGmAmCC*G*G*G*C*G*T*C*T*T*mCmCmCmCmG/iSp18//iSp18//3CholTEG/,
(SEQ ID NO: 516)
mGmGmGmUmGT*G*C*A*T*A*T*C*G*C*mAmGmUmCmG/iSp18//iSp18//3CholTEG/,
(SEQ ID NO: 596)
mGmAmGmCmAG*C*C*A*C*T*G*T*G*G*mAmUmGmCmA/iSp18//iSp18//3CholTEG/,
(SEQ ID NO: 614)
mCmCmUmGmCA*G*T*G*G*G*C*G*G*C*mCmGmUmGmA/iSp18//iSp18//3CholTEG/,
(SEQ ID NO: 829)
mCmCmAmGmCA*G*A*G*G*G*A*C*A*A*mUmGmAmGmA/iSp18//iSp18//3CholTEG/,
(SEQ ID NO: 830)
mCmCmCmAmGC*A*G*A*G*G*G*A*C*A*mAmUmGmAmG/iSp18//iSp18//3CholTEG/,
(SEQ ID NO: 831)
mUmCmCmCmAG*C*A*G*A*G*G*G*A*C*mAmAmUmGmA/iSp18//iSp18//3CholTEG/,
(SEQ ID NO: 852)
mAmGmCmCmAG*A*C*A*C*C*A*A*G*C*mAmCmUmUmU/iSp18//iSp18//3CholTEG/,
(SEQ ID NO: 856)
mCmCmCmCmAG*C*C*A*G*A*C*A*C*C*mAmAmGmCmA/iSp18//iSp18//3CholTEG/,
(SEQ ID NO: 875)
mUmUmGmGmGG*G*C*T*C*T*T*G*G*T*mUmGmUmCmC/iSp18//iSp18//3CholTEG/,
(SEQ ID NO: 876)
mCmUmUmGmGG*G*G*C*T*C*T*T*G*G*mUmUmGmUmC/iSp18//iSp18//3CholTEG/,
(SEQ ID NO: 878)
mCmAmCmUmUG*G*G*G*G*C*T*C*T*T*mGmGmUmUmG/iSp18//iSp18//3CholTEG/,
(SEQ ID NO: 879)
mGmCmAmCmUT*G*G*G*G*G*C*T*C*T*mUmGmGmUmU/iSp18//iSp18//3CholTEG/,
(SEQ ID NO: 891)
mCmCmUmUmAG*G*G*A*A*G*T*G*C*A*mCmUmUmGmG/iSp18//iSp18//3CholTEG/,
(SEQ ID NO: 901)
mCmAmCmUmGG*A*G*G*A*C*C*T*T*A*mGmGmGmAmA/iSp18//iSp18//3CholTEG/,
(SEQ ID NO: 905)
mCmAmAmGmCA*C*T*G*G*A*G*G*A*C*mCmUmUmAmG/iSp18//iSp18//3CholTEG/,
(SEQ ID NO: 908)
mAmUmUmCmAA*G*C*A*C*T*G*G*A*G*mGmAmCmCmU/iSp18//iSp18//3CholTEG/,
(SEQ ID NO: 909)
mUmAmUmUmCA*A*G*C*A*C*T*G*G*A*mGmGmAmCmC/iSp18//iSp18//3CholTEG/,
(SEQ ID NO: 912)
mUmGmAmUmAT*T*C*A*A*G*C*A*C*T*mGmGmAmGmG/iSp18//iSp18//3CholTEG/,
(SEQ ID NO: 913)
mCmUmGmAmUA*T*T*C*A*A*G*C*A*C*mUmGmGmAmG/iSp18//iSp18//3CholTEG/,
(SEQ ID NO: 954)
mGmUmCmUmCG*G*G*T*A*A*G*C*A*T*mCmCmUmCmG/iSp18//iSp18//3CholTEG/,
(SEQ ID NO: 980)
mGmGmCmGmCA*G*A*A*C*A*T*G*G*T*mGmUmCmAmU/iSp18//iSp18//3CholTEG/,
(SEQ ID NO: 1030)
mAmCmAmGmGC*C*C*C*C*C*A*G*A*A*mUmCmAmCmC/iSp18//iSp18//3CholTEG/,
(SEQ ID NO: 1079)
mAmGmGmGmUA*A*T*C*T*C*C*C*C*A*mGmGmAmCmA/iSp18//iSp18//3CholTEG/,
(SEQ ID NO: 1135)
mCmAmCmUmUG*G*T*G*A*A*C*T*T*G*mCmAmGmAmG/iSp18//iSp18//3CholTEG/,
(SEQ ID NO: 1147)
mGmUmUmUmCC*T*G*G*A*T*C*C*A*C*mUmUmGmGmU/iSp18//iSp18//3CholTEG/,
(SEQ ID NO: 1148)
mGmGmUmUmUC*C*T*G*G*A*T*C*C*A*mCmUmUmGmG/iSp18//iSp18//3CholTEG/,
(SEQ ID NO: 1154)
mCmUmGmGmAT*G*G*T*T*T*C*C*T*G*mGmAmUmCmC/iSp18//iSp18//3CholTEG/,
(SEQ ID NO: 1155)
mCmCmUmGmGA*T*G*G*T*T*T*C*C*T*mGmGmAmUmC/iSp18//iSp18//3CholTEG/,
(SEQ ID NO: 1156)
mGmCmCmUmGG*A*T*G*G*T*T*T*C*C*mUmGmGmAmU/iSp18//iSp18//3CholTEG/,
(SEQ ID NO: 1157)
mGmGmCmCmUG*G*A*T*G*G*T*T*T*C*mCmUmGmGmA/iSp18//iSp18//3CholTEG/,
(SEQ ID NO: 1161)
mAmGmUmUmGG*C*C*T*G*G*A*T*G*G*mUmUmUmCmC/iSp18//iSp18//3CholTEG/,
(SEQ ID NO: 1162)
mGmAmGmUmUG*G*C*C*T*G*G*A*T*G*mGmUmUmUmC/iSp18//iSp18//3CholTEG/,
(SEQ ID NO: 1163)
mGmGmAmGmUT*G*G*C*C*T*G*G*A*T*mGmGmUmUmU/iSp18//iSp18//3CholTEG/,
(SEQ ID NO: 1165)
mCmAmGmGmAG*T*T*G*G*C*C*T*G*G*mAmUmGmGmU/iSp18//iSp18//3CholTEG/,
(SEQ ID NO: 1166)
mUmCmAmGmGA*G*T*T*G*G*C*C*T*G*mGmAmUmGmG/iSp18//iSp18//3CholTEG/,
(SEQ ID NO: 1174)
mGmGmGmAmUG*A*C*T*C*A*G*G*A*G*mUmUmGmGmC/iSp18//iSp18//3CholTEG/,
(SEQ ID NO: 1189)
mGmGmUmGmUG*C*T*G*A*G*T*C*C*T*mGmGmGmAmU/iSp18//iSp18//3CholTEG/,
(SEQ ID NO: 1248)
mGmGmAmAmGG*A*A*T*G*A*G*G*G*T*mCmUmGmAmA/iSp18//iSp18//3CholTEG/,
(SEQ ID NO: 1259)
mCmAmAmCmAT*C*T*C*T*G*G*G*A*A*mGmGmAmAmU/iSp18//iSp18//3CholTEG/,
(SEQ ID NO: 1262)
mUmCmUmCmAA*C*A*T*C*T*C*T*G*G*mGmAmAmGmG/iSp18//iSp18//3CholTEG/,
(SEQ ID NO: 1265)
mCmAmUmUmCT*C*A*A*C*A*T*C*T*C*mUmGmGmGmA/iSp18//iSp18//3CholTEG/,
(SEQ ID NO: 1266)
mAmCmAmUmUC*T*C*A*A*C*A*T*C*T*mCmUmGmGmG/iSp18//iSp18//3CholTEG/,
(SEQ ID NO: 1267)
mAmAmCmAmUT*C*T*C*A*A*C*A*T*C*mUmCmUmGmG/iSp18//iSp18//3CholTEG/,
(SEQ ID NO: 1269)
mUmGmAmAmCA*T*T*C*T*C*A*A*C*A*mUmCmUmCmU/iSp18//iSp18//3CholTEG/,
(SEQ ID NO: 1275)
mGmAmGmAmGA*T*G*A*A*C*A*T*T*C*mUmCmAmAmC/iSp18//iSp18//3CholTEG/,
(SEQ ID NO: 1277)
mUmGmGmAmGA*G*A*T*G*A*A*C*A*T*mUmCmUmCmA/iSp18//iSp18//3CholTEG/,
(SEQ ID NO: 1278)
mCmUmGmGmAG*A*G*A*T*G*A*A*C*A*mUmUmCmUmC/iSp18//iSp18//3CholTEG/,
(SEQ ID NO: 1279)
mGmCmUmGmGA*G*A*G*A*T*G*A*A*C*mAmUmUmCmU/iSp18//iSp18//3CholTEG/,
(SEQ ID NO: 1292)
mCmAmUmGmGG*G*T*C*A*G*G*G*G*C*mUmGmGmAmG/iSp18//iSp18//3CholTEG/,
(SEQ ID NO: 1312)
mCmAmGmAmCC*C*T*G*A*G*T*C*C*A*mGmGmAmGmA/iSp18//iSp18//3CholTEG/,
(SEQ ID NO: 1334)
mGmGmUmCmAG*C*C*C*A*A*T*G*T*G*mGmGmGmGmA/iSp18//iSp18//3CholTEG/,
(SEQ ID NO: 1335)
mCmGmGmUmCA*G*C*C*C*A*A*T*G*T*mGmGmGmGmG/iSp18//iSp18//3CholTEG/,
(SEQ ID NO: 1337)
mCmAmCmGmGT*C*A*G*C*C*C*A*A*T*mGmUmGmGmG/iSp18//iSp18//3CholTEG/,
(SEQ ID NO: 1341)
mGmAmGmAmCA*C*G*G*T*C*A*G*C*C*mCmAmAmUmG/iSp18//iSp18//3CholTEG/,
(SEQ ID NO: 1342)
mAmGmAmGmAC*A*C*G*G*T*C*A*G*C*mCmCmAmAmU/iSp18//iSp18//3CholTEG/,
(SEQ ID NO: 1343)
mGmAmGmAmGA*C*A*C*G*G*T*C*A*G*mCmCmCmAmA/iSp18//iSp18//3CholTEG/,
(SEQ ID NO: 1344)
mAmGmAmGmAG*A*C*A*C*G*G*T*C*A*mGmCmCmCmA/iSp18//iSp18//3CholTEG/,
(SEQ ID NO: 1345)
mUmAmGmAmGA*G*A*C*A*C*G*G*T*C*mAmGmCmCmC/iSp18//iSp18//3CholTEG/,
(SEQ ID NO: 1346)
mCmUmAmGmAG*A*G*A*C*A*C*G*G*T*mCmAmGmCmC/iSp18//iSp18//3CholTEG/,
(SEQ ID NO: 1368)
mGmGmAmAmAT*T*G*T*T*C*C*C*A*G*mGmGmUmUmC/iSp18//iSp18//3CholTEG/,
(SEQ ID NO: 1397)
mGmAmGmAmCG*C*A*A*C*C*C*C*C*G*mCmCmCmUmG/iSp18//iSp18//3CholTEG/,
(SEQ ID NO: 1466)
mAmAmAmUmUT*G*G*G*T*C*A*G*A*G*mCmUmGmCmA/iSp18//iSp18//3CholTEG/,
(SEQ ID NO: 1474)
mCmUmGmGmGA*C*T*A*A*A*T*T*T*G*mGmGmUmCmA/iSp18//iSp18//3CholTEG/,
(SEQ ID NO: 1475)
mUmCmUmGmGG*A*C*T*A*A*A*T*T*T*mGmGmGmUmC/iSp18//iSp18//3CholTEG/,
(SEQ ID NO: 1624)
mCmAmCmGmGmUmGmUmUmAmGmAmGmGmGmGmUmGmGmU/iSp18//iSp18//3CholTEG/,

or a pharmaceutically acceptable salt thereof.

Embodiment 233. The synthetic oligonucleotide of embodiment 229, wherein the synthetic oligonucleotide comprises, consists essentially of, or consists of any one of SEQ ID NO: 112, SEQ ID NO: 315, SEQ ID NO: 830, SEQ ID NO: 891, SEQ ID NO: 1259, SEQ ID NO: 2010, SEQ ID NO: 2173, SEQ ID NO: 2625, SEQ ID NO: 2675, or SEQ ID NO: 3002, and/or wherein the synthetic oligonucleotide comprises, consists essentially of, or consists of any one of

(SEQ ID NO: 112)
mCmCmUmUmCC*C*T*G*C*C*T*G*C*T*mGmAmGmCmC/iSp18//
iSp18//3CholTEG/,
(SEQ ID NO: 315)
mCmCmCmAmCA*T*C*C*A*G*G*G*G*G*mGmUmCmUmU/iSp18//
iSp18//3CholTEG/,
(SEQ ID NO: 830)
mCmCmCmAmGC*A*G*A*G*G*G*A*C*A*mAmUmGmAmG/iSp18//
iSp18//3CholTEG/,
(SEQ ID NO: 891)
mCmCmUmUmAG*G*G*A*A*G*T*G*C*A*mCmUmUmGmG/iSp18//
iSp18//3CholTEG/,
or
(SEQ ID NO: 1259)
mCmAmAmCmAT*C*T*C*T*G*G*G*A*A*mGmGmAmAmU/iSp18//
iSp18//3CholTEG/,

or a pharmaceutically acceptable salt thereof.

Embodiment 234. The synthetic oligonucleotide of embodiment 231, wherein the nucleic acid comprises, consists essentially of, or consists of any one of SEQ ID NO; 3449, SEQ ID NO; 3670, SEQ ID NO: 3676, SEQ ID NO: 3704, SEQ ID NO: 3707, SEQ ID NO: 3749, SEQ ID NO: 3752, SEQ ID NO: 3998, SEQ ID NO: 4130, SEQ ID NO: 4868, SEQ ID NO: 5009, SEQ ID NO: 5024, SEQ ID NO: 5026, SEQ ID NO: 5376, SEQ ID NO: 5406, SEQ ID NO: 5411, SEQ ID NO: 5412, SEQ ID NO: 5425, SEQ ID NO: 5428, SEQ ID NO: 5431, SEQ ID NO: 5434, SEQ ID NO: 3996, SEQ ID NO: 3997, SEQ ID NO: 5437, SEQ ID NO: 5438, SEQ ID NO: 5443, SEQ ID NO: 5444, SEQ ID NO: 5445, SEQ ID NO: 5446, SEQ ID NO: 5447, SEQ ID NO: 5448, SEQ ID NO: 5454, SEQ ID NO: 5542, SEQ ID NO: 5588, SEQ ID NO: 5687, SEQ ID NO: 5879, SEQ ID NO: 5885, SEQ ID NO: 5901, SEQ ID NO: 5904, SEQ ID NO: 5940, SEQ ID NO: 5943, SEQ ID NO: 3998, SEQ ID NO: 6267, SEQ ID NO: 4868, SEQ ID NO: 7060, SEQ ID NO: 7070, SEQ ID NO: 7072, SEQ ID NO: 5376, SEQ ID NO: 7417, SEQ ID NO: 7422, SEQ ID NO: 7423, SEQ ID NO: 7431, SEQ ID NO: 7434, SEQ ID NO: 7437, SEQ ID NO: 7440, SEQ ID NO: 6153, SEQ ID NO: 6154, SEQ ID NO: 6155, SEQ ID NO; 4004, SEQ ID NOT 4005, SEQ ID NO: 4006, SEQ ID NO: 4007, SEQ ID NO: 6159, SEQ ID NO: 6160, SEQ ID NO: 6166, SEQ ID NO: 5910, SEQ ID NO; 5911, SEQ ID NO: 5912, SEQ ID NO; 5913, SEQ ID NO; 5918, SEQ ID NO; 5919, SEQ ID NO: 5920, SEQ ID NO: 5921, SEQ ID NO: 5922, SEQ ID NO: 5923, SEQ ID NO: 5929, SEQ ID NO: 6022, SEQ ID NO. 6068, SEQ ID NO; 6175, SEQ ID NO: 6396, SEQ ID NO: 6402, SEQ ID NO: 6430, SEQ ID NO: 6433, SEQ ID NO: 6475, SEQ ID NO; 6478, SEQ ID NO; 6724, SEQ ID NO: 6856, SEQ ID NO: 7594, SEQ ID NO: 7735, SEQ ID NO: 7750, SEQ ID NO: 7752, SEQ ID NO: 8102, SEQ ID NO: 8132, SEQ ID NO: 8137, SEQ ID NO: 8138, SEQ ID NO: 8151, SEQ ID NO 8154, SEQ ID NO: 8157, SEQ ID NO: 8160, SEQ ID NO: 6722, SEQ ID NO: 6723, SEQ ID NO: 6725, SEQ ID NO: 6730, SEQ ID NO: 6731, SEQ ID NO: 6732, SEQ ID NO: 6733, SEQ ID NO: 6734, SEQ ID NO: 6735, SEQ ID NO: 6741, or SEQ ID NO: 7516, and/or wherein the synthetic oligonucleotide comprises, consists essentially of, or consists of any one of

(SEQ ID NO: 3449)
mCmGmAmGA*G*G*A*T*C*T*G*A*T*mGmUmG/iSp18//iSp18//
3CholTEG/,
(SEQ ID NO: 3670)
mUmGmGmAG*C*T*G*A*T*T*G*C*C*mAmCmU/iSp18//iSp18//
3CholTEG/,
(SEQ ID NO: 3676)
mCmCmGmCA*G*T*G*G*A*G*C*T*G*mAmUmU/iSp18//iSp18//
3CholTEG/,
(SEQ ID NO: 3704)
mCmAmCmCC*A*G*C*G*C*T*C*A*T*mUmGmA/iSp18//iSp18//
3CholTEG/,
(SEQ ID NO: 3707)
mGmAmGmCA*C*C*C*A*G*C*G*C*T*mCmAmU/iSp18//iSp18//
3CholTEG/,
(SEQ ID NO: 3749)
mCmAmGmGT*G*C*A*C*G*G*T*G*T*mAmCmU/iSp18//iSp18//
3CholTEG/,
(SEQ ID NO: 3752)
mGmCmCmCA*G*G*T*G*C*A*C*G*G*mUmGmU/iSp18//iSp18//
3CholTEG/,
(SEQ ID NO: 3998)
mGmAmGmGT*C*A*G*A*G*G*G*A*A*mAmGmG/iSp18//iSp18//
3CholTEG/,
(SEQ ID NO: 4130)
mUmCmCmCC*C*T*G*A*G*T*C*A*C*mCmAmU/iSp18//iSp18//
3CholTEG/,
(SEQ ID NO: 4868)
mGmGmAmGG*C*T*G*A*A*G*C*A*C*mGmAmG/iSp18//iSp18//
3CholTEG/,
(SEQ ID NO: 5009)
mUmUmGmGG*A*G*G*C*C*A*A*G*G*mUmGmG/iSp18//iSp18//
3CholTEG/,
(SEQ ID NO: 5024)
mUmGmUmAA*T*C*C*C*A*G*C*A*C*mUmUmU/iSp18//iSp18//
3CholTEG/,
(SEQ ID NO: 5026)
mCmCmUmGT*A*A*T*C*C*C*A*G*C*mAmCmU/iSp18//iSp18//
3CholTEG/,
(SEQ ID NO: 5376)
mGmCmGmAmGmGmAmCmCmAmGmAmAmGmGmGmC/iSp18//iSp18//
3CholTEG/,
(SEQ ID NO: 5406)
mAmGmGmGmAmGmAmGmCmUmGmUmCmAmGmUmC/iSp18//iSp18//
3CholTEG/,
(SEQ ID NO: 5411)
mGmGmAmGmCmAmGmGmGmAmGmAmGmCmUmGmU/iSp18//iSp18//
3CholTEG/,
(SEQ ID NO: 5412)
mAmGmGmAmGmCmAmGmGmGmAmGmAmGmCmUmG/iSp18//iSp18//
3CholTEG/,
(SEQ ID NO: 5425)
mGmGmUmCmAmCmUmGmCmAmGmGmGmAmGmGmA/iSp18//iSp18//
3CholTEG/,
(SEQ ID NO: 5428)
mAmAmAmGmGmUmCmAmCmUmGmCmAmGmGmGmA/iSp18//iSp18//
3CholTEG/,
(SEQ ID NO: 5431)
mGmGmGmAmAmAmGmGmUmCmAmCmUmGmCmAmG/iSp18//iSp18//
3CholTEG/,
(SEQ ID NO: 5434)
mAmGmAmGmGmGmAmAmAmGmGmUmCmAmCmUmG/iSp18//iSp18//
3CholTEG/,
(SEQ ID NO: 3996)
mGmGmUmCmAmGmAmGmGmGmAmAmAmGmGmUmC/iSp18//iSp18//
3CholTEG/,
(SEQ ID NO: 3997)
mAmGmGmUmCmAmGmAmGmGmGmAmAmAmGmGmU/iSp18//iSp18//
3CholTEG/,
(SEQ ID NO: 5437)
mGmAmGmGmUmCmAmGmAmGmGmGmAmAmAmGmG/iSp18//iSp18//
3CholTEG/,
(SEQ ID NO: 5438)
mUmGmAmGmGmUmCmAmGmAmGmGmGmAmAmAmG/iSp18//iSp18//
3CholTEG/,
(SEQ ID NO: 5443)
mGmCmAmCmAmUmGmAmGmGmUmCmAmGmAmGmG/iSp18//iSp18//
3CholTEG/,
(SEQ ID NO: 5444)
mCmGmCmAmCmAmUmGmAmGmGmUmCmAmGmAmG/iSp18//iSp18//
3CholTEG/,
(SEQ ID NO: 5445)
mAmCmGmCmAmCmAmUmGmAmGmGmUmCmAmGmA/iSp18//iSp18//
3CholTEG/,
(SEQ ID NO: 5446)
mCmAmCmGmCmAmCmAmUmGmAmGmGmUmCmAmG/iSp18//iSp18//
3CholTEG/,
(SEQ ID NO: 5447)
mCmCmAmCmGmCmAmCmAmUmGmAmGmGmUmCmA/iSp18//iSp18//
3CholTEG/,
(SEQ ID NO: 5448)
mUmCmCmAmCmGmCmAmCmAmUmGmAmGmGmUmC/iSp18//iSp18//
3CholTEG/,
(SEQ ID NO: 5454)
mUmUmGmAmCmAmUmCmCmAmCmGmCmAmCmAmU/iSp18//iSp18//
3CholTEG/,
(SEQ ID NO: 5542)
mGmCmGmUmUmUmUmUmCmUmUmGmGmAmGmUmC/iSp18//iSp18//
3CholTEG/,
(SEQ ID NO: 5588)
mUmAmCmCmCmAmGmGmAmCmUmGmGmGmGmAmG/iSp18//iSp18//
3CholTEG/,

or a pharmaceutically acceptable salt thereof.

Embodiment 235. The synthetic oligonucleotide of embodiment 231, wherein the nucleic acid comprises, consists essentially of, or consists of any one of SEQ ID NO. 3998, SEQ ID NO. 3996, SEQ ID NO: 5437, SEQ ID NO: 5443, SEQ ID NO: 5588, SEQ ID NO: 3998, SEQ ID NO: 6153, SEQ ID NO: 4004, or SEQ ID NO: 7516, and/or wherein the synthetic oligonucleotide comprises, consists essentially of, or consists of any one of

(SEQ ID NO: 3998)
mGmAmGmGT*C*A*G*A*G*G*G*A*A*mAmGmG/iSp18//iSp18//
3CholTEG/,
(SEQ ID NO: 3996)
mGmGmUmCmAmGmAmGmGmGmAmAmAmGmGmUmC/iSp18//iSp18//
3CholTEG/,
(SEQ ID NO: 5437)
mGmAmGmGmUmCmAmGmAmGmGmGmAmAmAmGmG/iSp18//1Sp18//
3CholTEG/,
(SEQ ID NO: 5443)
mGmCmAmCmAmUmGmAmGmGmUmCmAmGmAmGmG/iSp18//iSp18//
3CholTEG/,
(SEQ ID NO: 5588)
mUmAmCmCmCmAmGmGmAmCmUmGmGmGmGmAmG/iSp18//iSp18//
3CholTEG/,

or a pharmaceutically acceptable salt thereof.

Embodiment 236. The synthetic oligonucleotide of any one of embodiments 229-235, wherein the nucleic acid sequence has a gap segment comprising five to 15 nucleosides, a 5′-wing segment comprising two to 10 linked nucleosides and a 3′-wing segment comprising two to 10 linked nucleosides, and wherein at least two consecutive nucleosides in at least one of the gap segment, the 5′-wing segment, the 3′-wing segment, or both the 5′-wing segment and the 3′-wing segment each comprise a 2′-O-methyl-modification.

Embodiment 237. The synthetic oligonucleotide of embodiment 236, wherein the 3′-wing segment comprises at least four consecutive nucleosides each comprising a 2′-O-methyl-modification.

Embodiment 238. The synthetic oligonucleotide of embodiment 236 or embodiment 237, wherein the 5′-wing segment comprises at least four consecutive nucleosides each comprising a 2′-O-methyl-modification.

Embodiment 239. The synthetic oligonucleotide of any one of embodiments 236-238, wherein the 3′-wing segment and the 5′-wing segment each comprise at least four consecutive nucleosides each comprising a 2′-O-methyl-modification.

Embodiment 240. The synthetic oligonucleotide any one of embodiments 229-239, wherein the nucleic acid sequence comprises a 2′-O-methyl-modification on at least one nucleoside.

Embodiment 241. The synthetic oligonucleotide of embodiment 240, wherein the 2′-O-methyl-modification is on at least 50% of the nucleosides.

Embodiment 242. The synthetic oligonucleotide of embodiment 240, wherein the 2′-O-methyl-modification is on every nucleoside.

Embodiment 243. The synthetic oligonucleotide of any one of embodiments 229-242, wherein the nucleic acid sequence comprises a phosphorothioate internucleoside linkage.

Embodiment 244. The synthetic oligonucleotide of embodiment 243, wherein at least 50% of internucleoside linkages are phosphorothioate internucleoside linkages.

Embodiment 245. The synthetic oligonucleotide of embodiment 243 or embodiment 244, wherein all internucleoside linkages are phosphorothioate internucleoside linkages.

Embodiment 246. The synthetic oligonucleotide of any one of embodiments 229-242, wherein the nucleic acid sequence does not comprise a phosphorothioate internucleoside linkage.

Embodiment 247. The synthetic oligonucleotide of any one of embodiments 229-239, wherein the nucleic acid sequence has a gap segment consisting of 10 linked nucleosides, a 5′-wing segment consisting of 4-5 linked nucleosides, and a 3′-wing segment consisting of 3-5 linked nucleosides, wherein the gap segment is positioned between the 5′-wing segment and the 3′-wing segment, and wherein two or more nucleosides in the 5′-wing segment, the 3′-wing segment, or both the 5′-wing segment and the 3′-wing segment comprise a modification.

Embodiment 248. The synthetic oligonucleotide of any one of embodiments 229-247, wherein the nucleic acid sequence further comprises a molecular species at one end of the nucleic acid sequence or at both ends of the nucleic acid sequence.

Embodiment 249. The synthetic oligonucleotide of embodiment 248, wherein the molecular species at both ends of the nucleic acid sequence are the same.

Embodiment 250. The synthetic oligonucleotide of embodiment 248, wherein the molecular species at both ends of the nucleic acid sequence are different.

Embodiment 251. The synthetic oligonucleotide of embodiment 248 or embodiment 250, wherein the molecular species is a cholesterol.

Embodiment 252. The synthetic oligonucleotide of embodiment 248 or embodiment 250, wherein the molecular species is a cholesteryl ester (N-cholesteryl-3-aminopropyl)-triethyleneglycol-glyceryl-1-O-phosphodiester (CholTEG).

Embodiment 253. The synthetic oligonucleotide of any one of embodiments 248-252, wherein the molecular species is directly attached to the nucleic acid sequence.

Embodiment 254. The synthetic oligonucleotide of any one of embodiment 248-253, wherein the molecular species is directly attached to the nucleic acid sequence through a covalent bond.

Embodiment 255. The synthetic oligonucleotide of any one of embodiments 248-254, wherein the molecular species is directly attached to the nucleic acid sequence through a phosphodiester, phosphorothioate, methylphosphonate or amide linkage.

Embodiment 256. The synthetic oligonucleotide of any one of embodiments 248-252, wherein the molecular species is indirectly attached to the nucleic acid sequence through a spacer.

Embodiment 257. The synthetic oligonucleotide of embodiment 256, wherein the spacer is or comprises a non-nucleotidic spacer.

Embodiment 258. The synthetic oligonucleotide of embodiment 256, wherein the spacer is or comprises a dSpacer, oligoethyleneglycol or alkane-diol.

Embodiment 259. The synthetic oligonucleotide of embodiment 256, wherein the spacer is or comprises a triethyleneglycol (spacer 9), hexaethylenegylcol (spacer 18) or butanediol.

Embodiment 260. The synthetic oligonucleotide of embodiment 256, wherein the spacer comprises a hexaethyleneglycol (spacer 18).

Embodiment 261. The synthetic oligonucleotide of embodiment 260, wherein the spacer comprises, consists essentially of, or consists of two hexaethyleneglycols.

Embodiment 262. A method of identifying synthetic oligonucleotides that decrease kallikrein-related peptidase (KLK) mRNA levels or KLK protein levels comprising.
(a) contacting a population of cells with a plurality of synthetic oligonucleotides complementary to or sufficiently complementary to one or more regions of a KLK gene and/or in one or more regions of a KLK gene product;
(b) culturing the population of cells to produce a population of cultured cells,
(c) measuring KLK mRNA levels or KLK protein levels in the population of cultured cells to obtain a value; and
(d) comparing the value in (c) with a reference level, wherein a value that is less than the reference level is indicative of a synthetic oligonucleotide that decreases KLK mRNA levels or KLK protein levels.

Embodiment 263. A method of identifying synthetic oligonucleotides that decrease kallikrein-related peptidase (KLK) mRNA levels or KLK protein levels comprising:
(a) contacting a population of cells with a plurality of spherical nucleic acids (SNAs), each SNA comprising a core and an oligonucleotide shell comprising a synthetic oligonucleotide complementary to or sufficiently complementary to one or more regions of a KLK gene and/or to one or more regions of a KLK gene product;
(b) culturing the population of cells to produce a population of cultured cells;
(c) measuring KLK mRNA levels or KLK protein levels in the population of cultured cells to obtain a value; and
(d) comparing the value in (c) with a reference level, wherein a value that is less than the reference level is indicative of a synthetic oligonucleotide that decreases KLK mRNA levels or KLK protein levels.

Embodiment 264. The method of embodiment 262 or embodiment 263, wherein the KLK mRNA is a KLK5 mRNA.

Embodiment 265. The method of embodiment 262 or embodiment 263, wherein the KLK protein is a KLK5 protein.

Embodiment 266. The method of embodiment 262 or embodiment 263, wherein the KLK mRNA is a KLK7 mRNA.

Embodiment 267. The method of embodiment 262 or embodiment 263, wherein the KLK protein is a KLK7 protein.

Embodiment 268. A library comprising a plurality of synthetic oligonucleotides, each synthetic oligonucleotide comprising a nucleic acid sequence complementary to or sufficiently complementary to a region of a kallikrein-related peptidase (KLK) gene and/or to a region of a KLK gene product, or a pharmaceutically acceptable salt thereof.

Embodiment 269. The library of embodiment 268, wherein the KLK gene is a KLK5 gene and/or the KLK gene product is a KLK5 gene product.

Embodiment 270. The library of embodiment 269, wherein the KLK gene is a KLK7 gene and/or the KLK gene product is a KLK7 gene product.

Embodiment 271. The library of any one of embodiment 268-270, wherein the region is within at least one of an exon, an intron, a 3′-untranslated region (3′-UTR), or a 5′-untranslated region (5′-UTR).

Embodiment 272. A library comprising a plurality of spherical nucleic acids (SNAs), each SNA comprising a synthetic oligonucleotide comprising a nucleic acid sequence complementary to or sufficiently complementary to a region of a kallikrein-related peptidase (KLK) gene and/or to a region of a KLK gene product.

Embodiment 273. The library of embodiment 272, wherein the KLK gene is a KLK5 gene.

Embodiment 274. The library of embodiment 272, wherein the KLK gene product is a KLK5 gene product.

Embodiment 275. The library of any one of embodiments 272-274, wherein the KLK gene is a KLK7 gene.

Embodiment 276. The library of any one of embodiment 272-274, wherein the KLK gene product is a KLK7 gene product.

Embodiment 277. The library of any one of embodiment 272-276, wherein the region is within at least one of an exon, an intron, a 3′-untranslated region (3′-UTR), or a 5′-untranslated region (5′-UTR).

Embodiment 278. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 4868, or a pharmaceutically acceptable salt thereof.

Embodiment 279. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of lG*lG*lA*G*G*/iMe-dC/*T*G*A*A*G*/iMe-dC/*A*/iMe-dC/*lG*lA*lG/iSp18//iSp18//3CholTEG/ (SEQ ID NO: 7842), or a pharmaceutically acceptable salt thereof.

Embodiment 280. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 1312, or a pharmaceutically acceptable salt thereof.

Embodiment 281. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of /5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), or a pharmaceutically acceptable salt thereof.

Embodiment 282. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 1312, and wherein the second synthetic oligonucleotide comprises the sequence of SEQ ID NO: 4868.

Embodiment 283. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises/5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), and wherein the second synthetic oligonucleotide comprises lG*lG*lA*G*G*/iMe-dC/*T*G*A*A*G*/iMe-dC/*A*/iMe-dC/*lG*lA*lG/iSp18//iSp18//3CholTEG/ (SEQ ID NO: 7842).

Embodiment 284. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 3998, or a pharmaceutically acceptable salt thereof.

Embodiment 285. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of moeG*moeAmoeG*moeGmoeT*/5-Me-moeC/moeA*moeGmoeA*moeG/7deazaOMG/*moeGmoeA*moeAmoeA*moeG*moeG/iSp18//iSp18//3CholTEG/ (SEQ ID NO: 7616), or a pharmaceutically acceptable salt thereof.

Embodiment 286. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 1312, and wherein the second synthetic oligonucleotide comprises the sequence of SEQ ID NO: 3998.

Embodiment 287. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises /5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), and wherein the second synthetic oligonucleotide comprises moeG*moeAmoeG*moeGmoeT*/5-Me-moeC/moeA*moeGmoeA*moeG/7deazaOMG/*moeGmoeA*moeAmoeA*moeG*moeG/iSp18//iSp18//3CholTEG/ (SEQ ID NO: 7616).

Embodiment 288. A synthetic oligonucleotide comprising a nucleic acid sequence complementary to or sufficiently complementary to a region of a kallikrein-related peptidase 5 (KLK5) gene and/or to a region of a KLK5 gene product, wherein the nucleic acid sequence comprises, consists essentially of, or consists of SEQ ID NO: 3118, or a pharmaceutically acceptable salt thereof.

Embodiment 289. A synthetic oligonucleotide comprising a nucleic acid sequence complementary to or sufficiently complementary to a region of a kallikrein-related peptidase 5 (KLK5) gene and/or to a region of a KLK5 gene product, wherein the synthetic oligonucleotide comprises, consists essentially of, or consists of lG*lA*lG*A*/iMe-dC/*G*/iMe-dC/*A*A*/iMe-dC/*/iMe-dC/*/iMe-dC/*/iMe-dC/*/iMe-dC/*G*/iMe-dC/*/iMe-dC/*/5-Me-lC/*lT*lG/iSp18//iSp18//3CholTEG/ (SEQ ID NO: 7836), or a pharmaceutically acceptable salt thereof.

Embodiment 290. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 3118, and wherein the second synthetic oligonucleotide comprises the sequence of SEQ ID NO: 3998.

Embodiment 291. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises lG*lA*lG*A*/iMe-dC/*G*/iMe-dC/*A*A*/iMe-dC/*/iMe-dC/*/iMe-dC/*/iMe-dC/*/iMe-dC/*G*/iMe-dC/*/iMe-dC/*/5-Me-lC/*lT*lG/iSp18//iSp18//3CholTEG/ (SEQ ID NO: 7836), and wherein the second synthetic oligonucleotide comprises moeG*moeAmoeG*moeGmoeT*/5-Me-moeC/moeA*moeGmoeA*moeG/7deazaOMG/*moeGmoeA*moeAmoeA*moeG*moeG/iSp18//iSp18//3CholTEG/ (SEQ ID NO: 7616).

Embodiment 292. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of lG*lA*lG*G*T*/iMe-dC/*A*G*A*G*/7deazaG/*G*A*A*lA*lG*lG/isp18//iSp18//3CholTEG/ (SEQ ID NO: 7616), or a pharmaceutically acceptable salt thereof.

Embodiment 293. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 3118, and wherein the second synthetic oligonucleotide comprises the sequence of SEQ ID NO: 3998.

Embodiment 294. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises lG*lA*lG*A*/iMe-dC/*G*/iMe-dC/*A*A*/iMe-dC/*/iMe-dC/*/iMe-dC/*/iMe-dC/*/iMe-dC/*G*/iMe-dC/*/iMe-dC/*/5-Me-lC/*lT*lG/iSp18//iSp18//3CholTEG/ (SEQ ID NO: 7836), and wherein the second synthetic oligonucleotide comprises lG*lA*lG*G*T*/iMe-dC/*A*G*A*G*/7deazaG/*G*A*A*lA*lG*lG/isp18//iSp18//3CholTEG/ (SEQ ID NO: 7616).

Embodiment 295. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of moeG*moeA*moeG*moeG*T*/iMe-dC/*A*G*A*G*/7deazaG/*G*A*A*moeA*moeG*moeG/iSp18//iSp18//3CholTEG/ (SEQ ID NO: 7616), or a pharmaceutically acceptable salt thereof.

Embodiment 296. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 1312, and wherein the second synthetic oligonucleotide comprises the sequence of SEQ ID NO: 3998.

Embodiment 297. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises /5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), and wherein the second synthetic oligonucleotide comprises moeG*moeA*moeG*moeG*T*/iMe-dC/*A*G*A*G*/7deazaG/*G*A*A*moeA*moeG*moeG/iSp18//iSp18//3CholTEG/ (SEQ ID NO: 7616).

Embodiment 298. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of /CholTEG//iSp18//iSp18/moeG*moeA*moeG*moeG*T*/iMe-dC/*A*G*A*G*/7deazaG/*G*A*A*moeA*moeG*moeG (SEQ ID NO: 7616), or a pharmaceutically acceptable salt thereof.

Embodiment 299. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 1312, and wherein the second synthetic oligonucleotide comprises the sequence of SEQ ID NO: 3998.

Embodiment 300. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises /5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), and wherein the second synthetic oligonucleotide comprises

(SEQ ID NO: 7616)
/CholTEG//iSp18//iSp18/moeG*moeA*moeG*moeG*T*/iMe-
dC/*A*G*A*G*/7deazaG/*G*A*A*moeA*moeG*moeG.

Embodiment 301. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of moeG*moeA*moeG*moeG*T*/iMe-dC/*A*G*A*G*/7deazaG/*G*A*A*moeA*moeG*moeG/iSp18//iSp18//3CholTEG/ (SEQ ID NO: 7616), or a pharmaceutically acceptable salt thereof.

Embodiment 302. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 3118, and wherein the second synthetic oligonucleotide comprises the sequence of SEQ ID NO: 3998.

Embodiment 303. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises lG*lA*lG*A*/iMe-dC/*G*/iMe-dC/*A*A*/iMe-dC/*/iMe-dC/*/iMe-dC/*/iMe-dC/*/iMe-dC/*G*/iMe-dC/*/iMe-dC/*/5-Me-lC/*lT*lG/iSp18//iSp18//3CholTEG/ (SEQ ID NO: 7836), and wherein the second synthetic oligonucleotide comprises moeG*moeA*moeG*moeG*T*/iMe-dC/*A*G*A*G*/7deazaG/*G*A*A*moeA*moeG*moeG/iSp18//iSp18//3CholTEG/ (SEQ ID NO: 7616).

Embodiment 304. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of /CholTEG//iSp18//iSp18/moeG*moeA*moeG*moeG*T*/iMe-dC/*A*G*A*G*/7deazaG/*G*A*A*moeA*moeG*moeG (SEQ ID NO: 7616), or a pharmaceutically acceptable salt thereof.

Embodiment 305. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 3118, and wherein the second synthetic oligonucleotide comprises the sequence of SEQ ID NO: 3998.

Embodiment 306. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises lG*lA*lG*A*/iMe-dC/*G*/iMe-dC/*A*A*/iMe-dC/*/iMe-dC/*/iMe-dC/*/iMe-dC/*/iMe-dC/*G*/iMe-dC/*/iMe-dC/*/5-Me-lC/*lT*lG/iSp18//iSp18//3CholTEG/ (SEQ ID NO: 7836), and wherein the second synthetic oligonucleotide comprises/CholTEG//iSp18//iSp18/moeG*moeA*moeG*moeG*T*/iMe-dC/*A*G*A*G*/7deazaG/*G*A*A*moeA*moeG*moeG (SEQ ID NO: 7616).

Embodiment 307. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of/CholTEG//iSp18//iSp18/G*lA*lG*G*T*/iMe-dC/*A*G*A*G*/7deazaG/*G*A*A*lA*lG*IG (SEQ ID NO: 7616), or a pharmaceutically acceptable salt thereof.

Embodiment 308. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 1312, and wherein the second synthetic oligonucleotide comprises the sequence of SEQ ID NO: 3998.

Embodiment 309. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises/5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), and wherein the second synthetic oligonucleotide comprises

(SEQ ID NO: 7616)
/CholTEG//iSp18//iSp18/1G*1A*1G*G*T*/iMe-dC/*A*G*
A*G*/7deazaG/*G*A*A*1A*1G*1G.

Embodiment 310. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 3118, and wherein the second synthetic oligonucleotide comprises the sequence of SEQ ID NO: 3998.

Embodiment 311. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises lG*lA*lG*A*/iMe-dC/*G*/iMe-dC/*A*A*/iMe-dC/*/iMe-dC/*/iMe-dC/*/iMe-dC/*% iMe-dC/*G*/iMe-dC/*/iMe-dC/*/5-Me-lC/*lT*lG/iSp18//iSp18//3CholTEG/ (SEQ ID NO: 7836), and wherein the second synthetic oligonucleotide comprises/CholTEG//iSp18//iSp18/G*lA*lG*G*T*/iMe-dC/*A*G*A*G*/7deazaG/*G*A*A*lA*lG*IG (SEQ ID NO: 7616).

Embodiment 312. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 5687, or a pharmaceutically acceptable salt thereof.

Embodiment 313. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of /5-Me-lC/*lG*lA*G*A*G*G*A*T*/iMe-dC/*T*G*A*T*lG*lT*lG/isp18//iSp18//3CholTEG/ (SEQ ID NO: 7583), or a pharmaceutically acceptable salt thereof.

Embodiment 314. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 1312, and wherein the second synthetic oligonucleotide comprises the sequence of SEQ ID NO: 5687.

Embodiment 315. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises /5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), and wherein the second synthetic oligonucleotide comprises/5-Me-lC/*lG*lA*G*A*G*G*A*T*/iMe-dC/*T*G*A*T*lG*lT*lG/isp18//iSp18//3CholTEG/ (SEQ ID NO: 7583).

Embodiment 316. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 5879, or a pharmaceutically acceptable salt thereof.

Embodiment 317. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of lT*lG*lG*A*G*/iMe-dC/*T*G*A*T*T*G*/iMe-dC/*/iMe-dC/*lA*/5-Me-lC/*lT/isp18//iSp18//3CholTEG/ (SEQ ID NO: 7592), or a pharmaceutically acceptable salt thereof.

Embodiment 318. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 1312, and wherein the second synthetic oligonucleotide comprises the sequence of SEQ ID NO: 5879.

Embodiment 319. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises/5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), and wherein the second synthetic oligonucleotide comprises lT*lG*lG*A*G*/iMe-dC/*T*G*A*T*T*G*/iMe-dC/*/iMe-dC/*lA*/5-Me-lC/*lT/isp18//iSp18//3CholTEG/ (SEQ ID NO: 7592).

Embodiment 320. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 5904, or a pharmaceutically acceptable salt thereof.

Embodiment 321. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of lG*lA*lG*/iMe-dC/*A*/iMe-dC/*/iMe-dC/*/iMe-dC/*A*G*/iMe-dC,/*G*/iMe-dC/*T*/5-Me-lC/*lA*T/isp18//iSp18//3CholTEG/ (SEQ ID NO: 7600), or a pharmaceutically acceptable salt thereof.

Embodiment 322. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 1312, and wherein the second synthetic oligonucleotide comprises the sequence of SEQ ID NO: 5904.

Embodiment 323. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises/5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), and wherein the second synthetic oligonucleotide comprises lG*lA*lG*/iMe-dC/*A*/iMe-dC/*/iMe-dC/*/iMe-dC/*A*G*/iMe-dC/*G*/iMe-dC/*T*/5-Me-lC/*lA*lT/isp18//iSp18//3CholTEG/ (SEQ ID NO: 7600).

Embodiment 324. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 5943, or a pharmaceutically acceptable salt thereof.

Embodiment 325. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of lG*/5-Me-lC/*/5-Me-lC/*/iMe-dC/*A*G*G*T*G*/iMe-dC/*A*/iMe-dC/*G*G*lT*lG*IT/isp18//iSp18//3CholTEG/ (SEQ ID NO: 7607), or a pharmaceutically acceptable salt thereof.

Embodiment 326. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 1312, and wherein the second synthetic oligonucleotide comprises the sequence of SEQ ID NO: 5943.

Embodiment 327. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises/5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), and wherein the second synthetic oligonucleotide comprises lG*/5-Me-lC/*/5-Me-lC/*/iMe-dC/*A*G*G*T*G*/iMe-dC/*A*/iMe-dC/*G*G*lT*lG*IT/isp18//iSp18//3CholTEG/ (SEQ ID NO: 7607).

Embodiment 328. A synthetic oligonucleotide comprising a nucleic acid sequence complementary to or sufficiently complementary to a region of a kallikrein-related peptidase 7 (KLK7) gene and/or to a region of a KLK7 gene product, wherein the nucleic acid sequence comprises, consists essentially of, or consists of SEQ ID NO: 6267, or a pharmaceutically acceptable salt thereof.

Embodiment 329. A synthetic oligonucleotide comprising a nucleic acid sequence complementary to or sufficiently complementary to a region of a kallikrein-related peptidase 7 (KLK7) gene and/or to a region of a KLK7 gene product, wherein the synthetic oligonucleotide comprises, consists essentially of, or consists of lT*/5-Me-lC/*/5-Me-lC/*/iMe-dC/*/iMe-dC/*/iMe-dC/*T*G*A*G*T*/iMe-dC/*A*/iMe-dC/*/5-Me-lC/*lA*lT/isp18//iSp18//3CholTEG/ (SEQ ID NO: 7623), or a pharmaceutically acceptable salt thereof.

Embodiment 330. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 1312, and wherein the second synthetic oligonucleotide comprises the sequence of SEQ ID NO: 6267.

Embodiment 331. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises /5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), and wherein the second synthetic oligonucleotide comprises lT*/5-Me-lC/*/5-Me-lC/*/iMe-dC/*/iMe-dC/*/iMe-dC/*T*G*A*G*T*/iMe-dC/*A*/iMe-dC/*/5-Me-lC/*lA*IT/isp18//iSp18//3CholTEG/ (SEQ ID NO: 7623).

Embodiment 332. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 3118, and wherein the second synthetic oligonucleotide comprises the sequence of SEQ ID NO: 5687.

Embodiment 333. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises lG*lA*lG*A*/iMe-dC/*G*/iMe-dC/*A*A*/iMe-dC/*/iMe-dC/*/iMe-dC/*/iMe-dC/*/iMe-dC/*G*/iMe-dC/*/iMe-dC/*/5-Me-lC/*lT*lG/iSp18//iSp18//3CholTEG/ (SEQ ID NO: 7836), and wherein the second synthetic oligonucleotide comprises/5-Me-lC/*lG*lA*G*A*G*G*A*T*/iMe-dC/*T*G*A*T*lG*lT*lG/isp18//iSp18//3CholTEG/ (SEQ ID NO: 7583).

Embodiment 334. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 3118, and wherein the second synthetic oligonucleotide comprises the sequence of SEQ ID NO: 5879.

Embodiment 335. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises lG*lA*lG*A*/iMe-dC/*G*/iMe-dC/*A*A*/iMe-dC/*/iMe-dC/*/iMe-dC/*/iMe-dC/*/iMe-dC/*G*/iMe-dC/*/iMe-dC/*/5-Me-lC/*lT*lG/iSp18//iSp18//3CholTEG/ (SEQ ID NO: 7836), and wherein the second synthetic oligonucleotide comprises lT*lG*lG*A*G*/iMe-dC/*T*G*A*T*T*G*/iMe-dC/*/iMe-dC/*lA*/5-Me-lC/*lT/isp18//iSp18//3CholTEG/ (SEQ ID NO: 7592).

Embodiment 336. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 3118, and wherein the second synthetic oligonucleotide comprises the sequence of SEQ ID NO: 5904.

Embodiment 337. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises lG*lA*lG*A*/iMe-dC/*G*/iMe-dC/*A*A*/iMe-dC/*/iMe-dC/*/iMe-dC/*/iMe-dC/*/iMe-dC/*G*/iMe-dC/*/iMe-dC/*/5-Me-lC/*lT*lG/iSp18//iSp18//3CholTEG/ (SEQ ID NO: 7836), and wherein the second synthetic oligonucleotide comprises lG*lA*lG*/iMe-dC/*A*/iMe-dC/*/iMe-dC/*/iMe-dC/*A*G*/iMe-dC/*G*/iMe-dC/*T*/5-Me-lC/*lA*lT/isp18//iSp18//3CholTEG/ (SEQ ID NO: 7600).

Embodiment 338. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 3118, and wherein the second synthetic oligonucleotide comprises the sequence of SEQ ID NO: 5943.

Embodiment 339. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises lG*lA*lG*A*/iMe-dC/*G*/iMe-dC/*A*A*/iMe-dC/*/iMe-dC/*/iMedC/*/iMe-dC/*/iMe-dC/*G*/iMe-dC*/iMe-dC/*/5-Me-lC/*lT*lG/iSp18//iSp18//3CholTEG/ (SEQ ID NO: 7836), and wherein the second synthetic oligonucleotide comprises lG*/5-Me-lC/*/5-Me-lC/*/iMe-dC/*A*G*G*T*G*/iMe-dC/*A*/iMe-dC/*G*G*lT*lG*IT/isp18//iSp18//3CholTEG/ (SEQ ID NO: 7607).

Embodiment 340. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 3118, and wherein the second synthetic oligonucleotide comprises the sequence of SEQ ID NO: 3998.

Embodiment 341. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises lG*lA*lG*A*/iMe-dC/*G*/iMe-dC/*A*A*/iMe-dC/*/iMe-dC/*/iMe-dC/*/iMe-dC/*% iMe-dC/*G*/iMe-dC/*/iMe-dC/*/5-Me-lC/*lT*lG/iSp18//iSp18//3CholTEG/ (SEQ ID NO: 7836), and wherein the second synthetic oligonucleotide comprises lG*lA*lG*G*T*/iMe-dC/*A*G*A*G*/7deazaG/*G*A*A*lA*lG*lG/isp18//iSp18//3CholTEG/ (SEQ ID NO: 7616).

Embodiment 342. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises the sequence of SEQ ID NO: 3118, and wherein the second synthetic oligonucleotide comprises the sequence of SEQ ID NO: 6267.

Embodiment 343. A spherical nucleic acid (SNA) comprising a core and an oligonucleotide shell, wherein the oligonucleotide shell comprises: a first synthetic oligonucleotide complementary to or sufficiently complementary to a region of a first kallikrein-related peptidase (KLK) gene and/or to a region of a first KLK gene product; and a second synthetic oligonucleotide complementary to or sufficiently complementary to a region of a second KLK gene and/or to a region of a second KLK gene product; wherein the first synthetic oligonucleotide comprises lG*lA*lG*A*/iMe-dC/*G*/iMe-dC/*A*A*/iMe-dC/*/iMe-dC/*/iMe-dC/*/iMe-dC/*/iMe-dC/*G*/iMe-dC/*/iMe-dC/*/5-Me-lC/*lT*lG/iSp18//iSp18//3CholTEG/ (SEQ ID NO: 7836), and wherein the second synthetic oligonucleotide comprises lT*/5-Me-lC/*/5-Me-lC/*/iMe-dC/*/iMe-dC/*/iMe-dC/*T*G*A*G*T*/iMe-dC/*A*/iMe-dC/*/5-Me-lC/*lA*lT/isp18//iSp18//3CholTEG/ (SEQ ID NO: 7623).

Embodiment 344. A spherical nucleic acid (SNA) comprising a core and an oligonucleotide shell that comprises a synthetic oligonucleotide, wherein the synthetic oligonucleotide comprises a nucleic acid sequence complementary to or sufficiently complementary to a region of a kallikrein-related peptidase (KLK) gene and/or to a region of a KLK gene product, or a pharmaceutically acceptable salt thereof.

### Sequences

Except where indicated otherwise, abbreviations used herein are defined according to Table 24.

TABLE 24
Abbreviations

| Abbreviation | Meaning |
|---|---|
| mA | 2′-O-Methyl Adenosine |
| mC | 2′-O-Methyl Cytidine |
| mG | 2′-O-Methyl Guanosine |
| mU | 2′-O-Methyl Uridine |
| A | Deoxyadenosine |
| C | Deoxycytidine |
| G | Deoxyguanosine |
| T | Deoxythymidine |
| moeA | 2′-O-Methoxyethoxy Adenosine |
| 5-Me-MOE-C | 2′-O-Methoxyethoxy-5-Methyl Cytidine |
| moeG | 2′-O-Methoxyethoxy Guanosine |
| moeT | 2′-O-Methoxyethoxy-5-Methyl Uridine |
| lA | LNA Adenosine |
| 5-Me-lC | LNA 5-Methyl Cytidine |
| lG | LNA Guanosine |
| lT | LNA Thymidine |
| iMe-dc | 5-Methyl deoxy Cytidine |
| 7deazaG | 7-Deaza deoxy Guanosine |
| 7deazaOMG | 7-deaza-2′-O-Methyl Guanosine |
| 3CholTEG | Cholesterol-triethyleneglycol (3′End) |
| CholTEG | Cholesterol-triethyleneglycol (5′End) |
| Toco | Tocopherol |
| 3ThioMC6-D | Thiol modifier C6 S-S |
| branch | Symmetrical Branching |
| Stearyl | Stearyl |
| iSp18 | Hexaethylene Glycol |
| * | Phosphorothioate internucleoside linkage |
| % Inhib | Percent gene inhibition |
| % Inhib. 1 | Percent gene inhibition, first measurement |
| % Inhib. 2 | Percent gene inhibition, second measurement |
| ASO | Antisense oligonucleotide |
| NR | None reported |

TABLE 25
Chemical names for LNAs used in sequences

| Abbreviation | Chemical Name |
|---|---|
| lA | (2′-O, 4′-C methylene)-Adenosine |
| 5-Me-lC | 5-methyl-(2′-O, 4′-C methylene)-Cytidine |
| lG | (2′-0, 4′-C methylene)-Guanosine |
| lT | 5-methyl-(2′-O, 4′-C methylene)-Uridine |

Abbreviations used in Tables 1, 2, 3 and 4: mA=2′-O-Methyl Adenosine; mC=2′-O-Methyl Cytidine; mG=2′-O-Methyl Guanosine; mU=2′-O-Methyl Uridine; 3CholTEG=3′ (N-cholesteryl-3-aminopropyl)-triethyleneglycol-glyceryl-1-O-phosphodiester; iSp18=Hexaethylene Glycol. A=deoxyadenosine; C=deoxycytidine, G=deoxyguanosine; T=deoxythymidine; *=phosphorothioate internucleoside linkage; NR=none reported; % Inhib. 1=percent gene inhibition, first measurement, % Inhib. 2=percent gene inhibition, second measurement; SNA=spherical nucleic acid; ASO=antisense oligonucleotide
“Seq. Start Site” within Tables 1-14 indicates the first nucleotide position of SEQ ID NO: 7575 or KLK oligonuclotides) or SEQ ID NO: 776 (for KLK7 oligonucleotides) to which the oligonucleotide is complementary, except as indicated with a †, in which case the nucleotide position is within SEQ ID NO: 7477 (for KLK5 oligonucleotides) or SEQ ID NO: 7478 (for KLK7 oligonucleotides).

Lengthy table referenced here
US20230340488A1-20231026-T00001
Please refer to the end of the specification for access instructions.

Lengthy table referenced here
US20230340488A1-20231026-T00002
Please refer to the end of the specification for access instructions.

Lengthy table referenced here
US20230340488A1-20231026-T00003
Please refer to the end of the specification for access instructions.

Lengthy table referenced here
US20230340488A1-20231026-T00004
Please refer to the end of the specification for access instructions.

Lengthy table referenced here
US20230340488A1-20231026-T00005
Please refer to the end of the specification for access instructions.

Lengthy table referenced here
US20230340488A1-20231026-T00006
Please refer to the end of the specification for access instructions.

Lengthy table referenced here
US20230340488A1-20231026-T00007
Please refer to the end of the specification for access instructions.

Lengthy table referenced here
US20230340488A1-20231026-T00008
Please refer to the end of the specification for access instructions.

Lengthy table referenced here
US20230340488A1-20231026-T00009
Please refer to the end of the specification for access instructions.

Lengthy table referenced here
US20230340488A1-20231026-T00010
Please refer to the end of the specification for access instructions.

Lengthy table referenced here
US20230340488A1-20231026-T00011
Please refer to the end of the specification for access instructions.

Lengthy table referenced here
US20230340488A1-20231026-T00012
Please refer to the end of the specification for access instructions.

Lengthy table referenced here
US20230340488A1-20231026-T00013
Please refer to the end of the specification for access instructions.

Lengthy table referenced here
US20230340488A1-20231026-T00014
Please refer to the end of the specification for access instructions.

Lengthy table referenced here
US20230340488A1-20231026-T00015
Please refer to the end of the specification for access instructions.

Lengthy table referenced here
US20230340488A1-20231026-T00016
Please refer to the end of the specification for access instructions.

Lengthy table referenced here
US20230340488A1-20231026-T00017
Please refer to the end of the specification for access instructions.

Lengthy table referenced here
US20230340488A1-20231026-T00018
Please refer to the end of the specification for access instructions.

Lengthy table referenced here
US20230340488A1-20231026-T00019
Please refer to the end of the specification for access instructions.

Lengthy table referenced here
US20230340488A1-20231026-T00020
Please refer to the end of the specification for access instructions.

Lengthy table referenced here
US20230340488A1-20231026-T00021
Please refer to the end of the specification for access instructions.

Lengthy table referenced here
US20230340488A1-20231026-T00022
Please refer to the end of the specification for access instructions.

Lengthy table referenced here
US20230340488A1-20231026-T00023
Please refer to the end of the specification for access instructions.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

LENGTHY TABLES
The patent application contains a lengthy table section. A copy of the table is available in electronic form from the USPTO web site (https://seqdata.uspto.gov/?pageRequest=docDetail&DocID=US20230340488A1). An electronic copy of the table will also be available from the USPTO upon request and payment of the fee set forth in 37 CFR 1.19(b)(3).

What is claimed is:

1. A synthetic oligonucleotide comprising a nucleic acid sequence complementary to or sufficiently complementary to a region of a kallikrein-related peptidase (KLK) gene and/or to a region of a KLK gene product, and at least one locked nucleic acid (LNA) modification or LNA modified nucleoside, or a pharmaceutically acceptable salt thereof.

2. The synthetic oligonucleotide of claim 1, wherein the KLK gene is a KLK5 gene.

3. The synthetic oligonucleotide of claim 1 or 2, wherein the KLK gene product is a KLK5 gene product.

4. The synthetic oligonucleotide of any one of claims 1-3, wherein the KLK gene is a KLK7 gene.

5. The synthetic oligonucleotide of any one of claims 1-4, wherein the KLK gene product is a KLK7 gene product.

6. A synthetic oligonucleotide comprising a nucleic acid sequence complementary to or sufficiently complementary to a region of a kallikrein-related peptidase (KLK) 7 gene and/or to a region of a KLK7 gene product, and at least one modification chosen from a base modification, a sugar modification or an internucleoside linkage modification, or a pharmaceutically acceptable salt thereof.

7. The synthetic oligonucleotide of any one of claims 1-6, wherein the synthetic oligonucleotide or the nucleic acid sequence is indirectly attached to a molecular species.

8. The synthetic oligonucleotide of claim 7, wherein the molecular species is indirectly attached to the 3′-end of the synthetic oligonucleotide or the nucleic acid sequence.

9. The synthetic oligonucleotide of claim 7, wherein the molecular species is indirectly attached to the 5′-end of the synthetic oligonucleotide or the nucleic acid sequence.

10. The synthetic oligonucleotide of claim 7 or 8, wherein the molecular species comprises, consists essentially of, or consists of a cholesterol or a tocopherol.

11. The synthetic oligonucleotide of any one of claims 9-10, wherein the molecular species comprises, consists essentially of, or consists of a cholesterol.

12. The synthetic oligonucleotide of any one of claims 7-11, wherein the molecular species comprises, consists essentially of, or consists of (N-cholesteryl-3-aminopropyl)-triethyleneglycol-glyceryl-1-O-phosphodiester (CholTEG).

13. The synthetic oligonucleotide of any one of claims 1-12, wherein the synthetic oligonucleotide further comprises a spacer.

14. The synthetic oligonucleotide of claim 13, wherein the spacer comprises, consists essentially of, or consists of an oligoethylene.

15. The synthetic oligonucleotide of claim 14, wherein the oligoethylene is hexaethyleneglycol (HEG).

16. The synthetic oligonucleotide of any one of claims 13-15, wherein the spacer comprises, consists essentially of, or consists of HEG and triethyleneglycol (TEG).

17. The synthetic oligonucleotide of any one of claims 13-16, wherein the spacer comprises, consists essentially of, or consists of hexa(ethyleneglycol)phosphodiester-hexa(ethyleneglycol)phosphodiester (HEG-HEG).

18. The synthetic oligonucleotide of any one of claims 13-17, wherein the molecular species is indirectly attached to the synthetic oligonucleotide or the nucleic acid sequence through the spacer.

19. The synthetic oligonucleotide of any one of claims 1-18, wherein the synthetic oligonucleotide comprises at least one phosphorothioate internucleoside linkage.

20. The synthetic oligonucleotide of any one of claims 1-18, wherein the synthetic oligonucleotide comprises two or more phosphorothioate internucleoside linkages, or wherein each internucleoside linkage of the synthetic oligonucleotide is a phosphorothioate internucleoside linkage.

21. The synthetic oligonucleotide of any one of claims 1-20, wherein the synthetic oligonucleotide comprises at least two modifications or modified nucleoside(s).

22. The synthetic oligonucleotide of claim 21, wherein the at least two modifications or modified nucleosides are at least one of a modification or modified nucleoside chosen from a 2′-O-methyl modification (2′-O-Me) or 2′-O-Me modified nucleoside, a 2′-O-methoxyethyl (2′-MOE) modification or 2′-MOE modified nucleoside, a 2′-O-methoxyethoxy-5-methyl (5-Me-MOE) modification or 5-Me-MOE modified nucleoside, an LNA modification or LNA modified nucleoside, a 5-methyl (5-Me) modification or 5-Me modified nucleoside, a 5-methyl LNA modified nucleoside, a 7-deaza modification or 7-deaza modified nucleoside, and a 7-deaza-2′-O-methyl (7deazaOM) modification or 7deazaOM modified nucleoside.

23. The synthetic oligonucleotide of any one of claims 1-22, wherein the synthetic oligonucleotide further comprises at least one modification or modified nucleoside chosen from a 5-Me modification or a 5-Me modified nucleoside, a 7-deaza modification or 7-deaza modified nucleoside and a phosphorothioate internucleoside linkage.

24. The synthetic oligonucleotide of any one of claims 1-23, wherein synthetic oligonucleotide comprises an LNA modification or an LNA modified nucleoside, a 5-Me modification or 5-Me modified nucleoside, and a phosphorothioate internucleoside linkage.

25. The synthetic oligonucleotide of any one of claims 1-23, wherein the synthetic oligonucleotide comprises five 5-Me modified nucleosides and six LNA modified nucleosides.

26. The synthetic oligonucleotide of any one of claims 1-23, wherein the synthetic oligonucleotide comprises an LNA modification or an LNA modified nucleoside, a 5-Me modification or 5-Me modified nucleoside, a 7-deaza modification or 7-deaza modified nucleoside, and a phosphorothioate internucleoside linkage.

27. The synthetic oligonucleotide of any one of claims 1-23, wherein the synthetic oligonucleotide comprises one 5-Me modification or 5-Me modified nucleoside, one 7-deaza modification or 7-deaza modified nucleoside and six LNA modifications or LNA modified nucleosides.

28. The synthetic oligonucleotide of any one of claim 1-27, wherein the LNA modified nucleoside comprises, consists essentially of, or consists of (2′-O, 4′-C methylene)-Adenosine, 5-methyl-(2′-O, 4′-C methylene)-Cytidine, (2′-O, 4′-C methylene)-Guanosine, or 5-methyl-(2′-O, 4′-C methylene)-Uridine.

29. The synthetic oligonucleotide of any one of claims 1-28, wherein the region is within an exon.

30. The synthetic oligonucleotide of any one of claims 1-29, wherein the region is within exon 6 of a KLK5 gene or a KLK5 gene product.

31. The synthetic oligonucleotide of any one of claims 1-30, wherein the region is within exon 5 of a KLK7 gene or a KLK7 gene product.

32. The synthetic oligonucleotide of any one of claims 1-29, wherein the region is within exon 1, exon 3, exon 4 or exon 6 of a KLK7 gene or a KLK7 gene product.

33. The synthetic oligonucleotide of any one of claims 1-29, wherein the region is within both exons 5 and 6 of a KLK7 gene or a KLK7 gene product.

34. The synthetic oligonucleotide of any one of claims 1-33, wherein the region is within a 5′-untranslated region (UTR) or a 3′-UTR.

35. The synthetic oligonucleotide of any one of claims 1-33, wherein the region is within a coding sequence.

36. The synthetic oligonucleotide of any one of claims 1-35, wherein the nucleic acid sequence is 10 to 30 nucleobases or nucleosides in length.

37. The synthetic oligonucleotide of any one of claims 1-35, wherein the nucleic acid sequence is 15 to 22 nucleobases or nucleosides in length.

38. The synthetic oligonucleotide of any one of claims 1-35, wherein the nucleic acid sequence is 17 nucleobases or nucleosides in length.

39. The synthetic oligonucleotide of any one of claims 1-35, wherein the nucleic acid sequence is 20 nucleobases or nucleosides in length.

40. The synthetic oligonucleotide of any one of claims 1-39, wherein the nucleic acid sequence comprises, consists essentially of, or consists of GAGGTCAGAGGGAAAGG (SEQ ID NO. 3998).

41. The synthetic oligonucleotide of any one of claims 1-39, wherein the synthetic oligonucleotide comprises, consists essentially of, or consists of /CholTEG//iSp18//iSp18/lG*lA*lG*G*T*/iMe-dC/*A*G*A*G*/7deazaG/*G*A*A*lA*lG*lG (SEQ ID NO: 7616), wherein lA, lG, A, T, G, iMe-dC, 7deazaG, *, iSp18 and CholTEG are defined as in Table 24.

42. The synthetic oligonucleotide of any one of claims 1-39, wherein the nucleic acid sequence comprises, consists essentially of, or consists of CAGACCCTGAGTCCAGGAGA (SEQ ID NO: 1312).

43. The synthetic oligonucleotide of any one of claims 1-39, wherein the synthetic oligonucleotide comprises, consists essentially of, or consists of /5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), wherein 5-Me-lC, lA, lG, A, T, G, iMe-dC, *, iSp18 and 3CholTEG are defined as in Table 24.

44. The synthetic oligonucleotide of any one of claims 1-39, wherein the nucleic acid sequence comprises, consists essentially of, or consists of GAGACGCAACCCCCGCCCTG (SEQ ID NO: 3118).

45. The synthetic oligonucleotide of any one of claims 1-39, wherein the synthetic oligonucleotide comprises, consists essentially of, or consists of lG*lA*lG*A*/iMe-dC/*G*/iMe-dC/*A*A*/iMe-dC/*/iMe-dC/*/iMe-dC/*/iMe-dC/*/iMe-dC/*G*/iMe-dC/*/iMe-dC/*/5-Me-lC/*lT*lG/iSp18//iSp18//3CholTEG/ (SEQ ID NO: 7836), wherein 5-Me-lC, lA, lG, lT, A, G, iMe-dC, *, iSp18, and 3CholTEG are defined as in Table 24.

46. A pharmaceutical composition comprising the synthetic oligonucleotide, or the pharmaceutically acceptable salt thereof, of any one of claims 1-45.

47. A spherical nucleic acid (SNA) comprising a core and an oligonucleotide shell, wherein the oligonucleotide shell comprises:
a first synthetic oligonucleotide comprising a first nucleic acid sequence complementary to or sufficiently complementary to a region of a first kallikrein-related peptidase (KLK) gene that is a KLK5 gene and/or to a region of a first KLK gene product that is a KLK5 gene product; and
a second synthetic oligonucleotide comprising a second nucleic acid sequence complementary to or sufficiently complementary to a region of a second KLK gene that is a KLK7 gene and/or to a region of a second KLK gene product that is a KLK7 gene product.

48. The SNA of claim 47, wherein the region of the first KLK gene and/or the region of the first KLK gene product is within exon 6 of a KLK5 gene or a KLK5 gene product.

49. The SNA of claim 47, wherein the region of the second KLK gene and/or the region of the second KLK gene product is within exon 5 of a KLK7 gene or a KLK7 gene product.

50. The SNA of claim 47, wherein the region of the first KLK gene and/or the region of the first KLK gene product is within exon 6 of a KLK5 gene or a KLK5 gene product and the region of the second KLK gene and/or the region of the second KLK gene product is within exon of a KLK7 gene or a KLK7 gene product.

51. The SNA of any one of claims 47-50, wherein the first synthetic oligonucleotide is a synthetic oligonucleotide of any one of claims 1-45 and the second synthetic oligonucleotide is a synthetic oligonucleotide of any one of claims 1-45.

52. The SNA of any one of claims 47-51, wherein the first synthetic oligonucleotide or nucleic acid sequence is indirectly attached to a first molecular species and the second synthetic oligonucleotide or nucleic acid sequence is indirectly attached to a second molecular species.

53. The SNA of claim 52, wherein the first molecular species is indirectly attached to one end of the first synthetic oligonucleotide or nucleic acid sequence and the second molecular species is indirectly attached to one end of the second synthetic oligonucleotide or nucleic acid sequence.

54. The SNA of claim 52 or 53, wherein the first molecular species is indirectly attached to the 3′-end of the first synthetic oligonucleotide or nucleic acid sequence and the second molecular species is indirectly attached to the 5′-end of the second synthetic oligonucleotide or nucleic acid sequence.

55. The SNA of any one of claims 52-54, wherein the first synthetic oligonucleotide is anchored to the surface of the core through the first molecular species and the second synthetic oligonucleotide is anchored to the surface of the core through the second molecular species.

56. The SNA of any one of claims 52-55, wherein the oligonucleotide shell comprises a plurality of the first synthetic oligonucleotides to form a first population of synthetic oligonucleotides and a plurality of the second synthetic oligonucleotides to form a second population of synthetic oligonucleotides, wherein each of the first synthetic oligonucleotides is anchored to the surface of the core through the first molecular species and each of the second synthetic oligonucleotides is anchored to the surface of the core through the second molecular species.

57. The SNA of any one of claims 47-56, wherein the first nucleic acid sequence comprises, consists essentially of, or consists of CAGACCCTGAGTCCAGGAGA (SEQ ID NO: 1312), and wherein the second nucleic acid sequence comprises, consists essentially of, or consists of GAGGTCAGAGGGAAAGG (SEQ ID NO: 3998).

58. The SNA of any one of claims 47-57, wherein the first synthetic oligonucleotide comprises, consists essentially of, or consists of /5-Me-lC/*lA*lG*A*/iMe-dC/*/iMe-dC/*/iMe-dC/*T*G*A*G*T*/iMe-dC/*/iMe-dC/*A*G*G*lA*lG*lA/iSp18//iSp18//3CholTEG/ (SEQ ID NO: 7834), and wherein the second synthetic oligonucleotide comprises, consists essentially of, or consists of/CholTEG//iSp18//iSp18/lG*lA*lG*G*T*/iMe-dC/*A*G*A*G*/7deazaG/*G*A*A*lA*lG*IG (SEQ ID NO: 7616), wherein 5-Me-lC, lA, lG, A, T, G, iMe-dC, 7deazaG, *, iSp18, CholTEG and 3CholTEG are defined as in Table 24.

59. The SNA of any one of claims 47-58, wherein the first synthetic oligonucleotide comprises, consists essentially of, or consists of the synthetic oligonucleotide of claim A43 and wherein the second synthetic oligonucleotide comprises, consists essentially of, or consists of the synthetic oligonucleotide of claim A41.

60. The SNA of any one of claims 47-56, wherein the first nucleic acid sequence comprises, consists essentially of, or consists of GAGACGCAACCCCCGCCCTG (SEQ ID NO: 3118), and wherein the second nucleic acid sequence comprises, consists essentially of, or consists of GAGGTCAGAGGGAAAGG (SEQ ID NO: 3998).

61. The SNA of any one of claims 47-56 and 60, wherein the first synthetic oligonucleotide comprises, consists essentially of, or consists of lG*lA*lG*A*/iMe-dC/*G*/iMe-dC/*A*A*/iMe-dC/*/iMe-dC/*/iMe-dC/*/iMe-dC/*/iMe-dC/*G*/iMe-dC/*/iMe-dC,*/5-Me-lC/*lT*lG/iSp18//iSp18;/3CholTEG/ (SEQ ID NO: 7836), and wherein the second synthetic oligonucleotide comprises, consists essentially of, or consists of /CholTEG//iSp18//iSp18/G*lA*lG*G*T*/iMe-dC/*A*G*A*G*/7deazaG/*G*A*A*lA*lG*lG (SEQ ID NO: 7616), wherein lG, lA, lT, 5-Me-lC, A, G, T, iMe-dC, 7deazaG, *, iSp18, CholTEG and 3CholTEG are defined as in Table 24.

62. The SNA of any one of claims 47-56, wherein the first synthetic oligonucleotide comprises, consists essentially of, or consists of the synthetic oligonucleotide of claim 45 and wherein the second synthetic oligonucleotide comprises, consists essentially of, or consists of the synthetic oligonucleotide of claim 41.

63. The SNA of any one of claims 47-62, wherein the core is a hollow core or solid core.

64. The SNA of claim 63, wherein the hollow core is a liposome core.

65. The SNA of any one of claims 47-64, wherein the oligonucleotide shell comprises, consists essentially of, or consists of 20 to 50 total first synthetic oligonucleotides and second synthetic oligonucleotides.

66. The SNA of any one of claims 47-65, wherein the oligonucleotide shell comprises, consists essentially of, or consists of 25 to 35 total first synthetic oligonucleotides and second synthetic oligonucleotides.

67. The SNA of any one of claims 47-66, wherein the oligonucleotide shell comprises, consists essentially of, or consists of or about 30 total first synthetic oligonucleotides and second synthetic oligonucleotides.

68. The SNA of claim 67, wherein the oligonucleotide shell comprises, consists essentially of, or consists of or about 15 first synthetic oligonucleotides and of or about 15 second synthetic oligonucleotides.

69. The SNA of any one of claims 56-68, wherein the core is a liposome core comprising lipid molecules, and wherein the first population of synthetic oligonucleotides and the second population of synthetic oligonucleotides are at a molar ratio of or about 50 to 1 of lipid molecules to the first population of synthetic oligonucleotides and the second population of synthetic oligonucleotides.

70. The SNA of any one of claims 56-68, wherein the core is a liposome core comprising or consisting of lipid molecules, and wherein the first population of synthetic oligonucleotides and the second population of synthetic oligonucleotides are at a molar ratio of or about 50 to 0.5 to 0.5 of lipid molecules to the first population of synthetic oligonucleotides to the second population of synthetic oligonucleotides.

71. A pharmaceutical composition comprising the SNA of any one of claims 47-70.

72. A method of decreasing kallikrein-related peptidase (KLK) mRNA levels in a cell, the method comprising:
contacting a cell comprising a KLK gene and/or a KLK gene product, with a synthetic oligonucleotide of any one of claims A1-A45, an SNA of any one of claims 47-70, or a pharmaceutical composition of claim 46 or 71, to decrease KLK mRNA levels in the cell relative to a reference level.

73. The method of claim 72, wherein the KLK mRNA levels are KLK5 mRNA levels, KLK7 mRNA levels, or both KLK5 mRNA levels and KLK7 mRNA levels.

74. The method of claim 72 or 73, wherein the cell comprises a KLK5 gene, a KLK7 gene, or a KLK5 gene and a KLK7 gene.

75. The method of claim 73, wherein the decreased KLK5 mRNA levels, KLK7 mRNA levels, or KLK5 mRNA levels and KLK7 mRNA levels result in decreased KLK5 protein levels, KLK7 protein levels, or both KLK5 protein levels and KLK7 protein levels in the cell relative to a reference level.

76. The method of any one of claims 72-75, wherein the cell is a skin cell.

77. The method of any one of claims 72-75, wherein the cell is at least one of an epithelial cell, a dermal cell, a keratinocyte, an immune cell, a basal cell, an inner root sheath cell, an external root sheath cell, a sebaceous gland cell, and a sweat gland cell.

78. The method of any one of claims 72-75, wherein the cell is a keratinocyte.

79. A method of treating a disease or disorder in a subject comprising:
administering to a subject in order to treat the disease or disorder in the subject an effective amount of a synthetic oligonucleotide of any one of claims 1-45, an SNA of any one of claims 47-70, or a pharmaceutical composition of claim 46 or 71.

80. The method of claim 79, wherein the disease or disorder is an ichthyosis disease or disorder.

81. The method of claim 79 or 80, wherein the subject has mutations in two serine peptidase inhibitor, Kazal type 5 (SPINK5) alleles.

82. The method of any one of claims 79-81, wherein the disease or disorder is a chronic skin disease or disorder.

83. The method of any one of claims 79-81, wherein the disease or disorder is Netherton syndrome (NS) or atopic dermatitis.

84. The method of any one of claims 79-81, wherein the disease or disorder is NS.

85. The method of any one of claims 79-84, wherein the decrease in KLK5 mRNA levels, KLK7 mRNA levels, or in both KLK5 mRNA levels and KLK7 mRNA levels and/or the decrease in KLK5 protein levels, KLK7 protein levels, or both KLK5 protein levels and KLK7 protein levels in the subject is relative to a reference level.

86. The method of claim 85, wherein the KLK5 and/or KLK7 mRNA and/or protein levels are decreased in one or more cells in the subject, and wherein the one or more cells in the subject are at least one of a skin cell, an epithelial cell, a dermal cell, a keratinocyte, an immune cell, a basal cell, an inner root sheath cell, an external root sheath cell, a sebaceous gland cell, and a sweat gland cell.

87. The method of claim 85, wherein the KLK5 and/or KLK7 mRNA and/or protein levels are decreased in a keratinocyte.

88. The method of any one of claims 79-87, wherein the synthetic oligonucleotide, the SNA, or the pharmaceutical composition ameliorate or eliminate one or more symptoms or conditions associated with the disease or disorder in the subject, and wherein the symptom or condition is at least one of erythroderma, xeroderma, desquamation, pruritus, skin infections, septicemia, inflammation, ichthyosis linearis circumflexa, bamboo hair, eczema, asthma, allergies, hypernatremia, and dehydration.

89. The method of any one of claims 79-88, further comprising the administration of a second therapeutic agent.
